(19) United States
(12) Patent Application Publication
(10) Pub. No.: US 2015/0144846 A1
(43) Pub. Date: May 28, 2015

(54) ORGANIC SEMICONDUCTOR

(71) Applicant: MERCK PATENT GmbH, Darmstadt (DE)

(72) Inventors: Lana Nanson, Southampton (GB); Nicolas Blouin, Southampton (GB); William Mitchell, Chandler's Ford (GB); Steven Tierney, Southampton (GB); Toby Cull, Romsey (GB)

(73) Assignee: MERCK PATENT GmbH, Darmstadt (DE)

(21) Appl. No.: 14/405,747

(22) PCT Filed: May 6, 2013
(86) PCT No.: PCT/EP2013/001332

§ 371 Date: Dec. 4, 2014

(30) Foreign Application Priority Data
Jun. 4, 2012 (EP) 12004246.0

(51) Int. Cl. C08G 61/12; H01L 51/00; C07F 7/22; C07D 471/04

(52) U.S. Cl. CPC C08G 61/126; C07D 471/04; H01L 51/0043; H01L 51/0036; H01L 51/0046; C07F 7/2212; H01L 51/4253
USPC 252/500; 546/122; 526/240; 549/3

(57) ABSTRACT

The invention relates to novel compounds containing one or more units derived from 1,5-disubstituted-1,5-dihydro-[1,5]naphthyridine-2,6-dione, to methods for their preparation and educts or intermediates used therein, to mixtures and formulations containing them, to the use of the compounds, mixtures and formulations as organic semiconductors in organic electronic (OE) devices, especially in organic photovoltaic (OPV) devices and organic photodetectors (OPD), and to OE, OPV and OPD devices comprising these compounds, mixtures or formulations.

### TECHNICAL FIELD

The invention relates to novel compounds containing one or more units derived from 1,5-disubstituted-1,5-dihydro-[1,5]naphthyridine-2,6-dione, to methods for their preparation and educts or intermediates used therein, to mixtures and formulations containing them, to the use of the compounds, mixtures and formulations as organic semiconductors in organic electronic (OE) devices, especially in organic photovoltaic (OPV) devices and organic photodetectors (OPD), and to OE, OPV and OPD devices comprising these compounds, mixtures or formulations.

### BACKGROUND

In recent years, there has been development of organic semiconducting (OSC) materials in order to produce more versatile, lower cost electronic devices. Such materials find application in a wide range of devices or apparatus, including organic field effect transistors (OFETs), organic light emitting diodes (OLEDs), photodetectors, organic photovoltaic (OPV) cells, organic photodetectors (OPD), sensors, memory elements and logic circuits to name just a few. The organic semiconducting materials are typically present in the electronic device in the form of a thin-film layer.

The performance of OFET devices is principally based upon the charge carrier mobility of the semiconducting material and the current on/off ratio, so the ideal semiconductor should have a low conductivity in the off state, combined with a high charge carrier mobility (>1×10⁻³ cm² V⁻¹ s⁻¹). In addition, it is important that the semiconducting material is relatively stable to oxidation i.e. it has a high ionisation potential, as oxidative doping leads to reduced device performance, for example increased off current and threshold voltage shift. Further requirements for the semiconducting material to have include good processability, especially for large-scale production of thin-film layers and desired patterns, and high stability, thin-film uniformity and integrity of the organic semiconductor layer.

Polymers have found use in OPVs as they allow devices to be manufactured by solution-processing techniques such as spin casting, dip coating or ink jet printing. Solution processing can be carried out cheaper and on a larger scale compared to the evaporative techniques used to make inorganic thin film devices. Currently, polymer based devices are achieving efficiencies over 8%.

The conjugated polymer serves as the main absorber of the solar energy in the bulk-heterojunction blend layer and therefore a low band gap is a basic requirement of the ideal polymer design to absorb the maximum of the solar spectrum.

A commonly used strategy to narrow the band gap of polymers is to utilise an alternating copolymer consisting of both electron rich donor units and electron deficient acceptor units within the polymer backbone. However, the ideal polymer, e.g. high efficiency, facile synthesis and scalable, has yet to be found.

Thus there is still a need for organic semiconducting (OSC) polymers which are easy to synthesize, especially by methods suitable for mass production, show good structural organization and film-forming properties, exhibit good electronic properties, especially a high charge carrier mobility, a good processability, especially a high solubility in organic solvents, and high stability in air. Especially for use in OPV cells, there is a need for OSC materials having a low bandgap, which enable improved light harvesting by the photoactive layer and can lead to higher cell efficiencies, compared to the polymers from prior art.

It was an aim of the present invention to provide compounds and polymers for use in OFET and OPV devices that do not have the drawbacks of prior art materials as described above, and do especially show good solubility in organic solvents, high charge carrier mobility, improved Voc and power conversion efficiency. Another aim of the invention was to extend the pool of organic semiconducting materials available to the expert.

The inventors of the present invention have found that one or more of the above aims can be achieved by providing compounds having a divalent unit based on 1,5-disubstituted-1,5-dihydro-[1,5]naphthyridine-2,6-dione, incorporating fused lactam rings into two six-membered rings. The ring system incorporating two fused six-membered rings leads to an alternative solubility and morphology profile, which has an impact on the compounds' electrical properties, including an increase in the HOMO energy level compared to DPP (diketopyrrolopyrrole) materials from prior art, and consequently their OFET and/or OPV device performance.

H. Rapoport, A. D. Batcho, J. Org. Chem. 1963. 28, 1753 disclose 1,5-dihydro-1,5-dimethyl-[1,5]naphthyridine-2,6-dione and its 3-ethyl derivative as drug candidates for use in the pharmaceutical industry. However, the compounds as disclosed in the present invention and as claimed hereinafter and their use as organic semiconductors have not been disclosed or suggested in prior art so far.

### SUMMARY

The invention relates to compounds comprising one or more divalent units of formula I

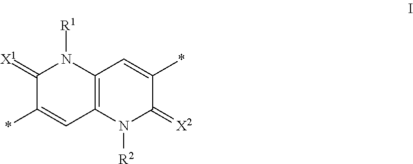

wherein
X¹ and X² independently of each other denote O or S,
R¹ and R² independently of each other denote H, straight-chain, branched or cyclic alkyl with 1 to 30 C atoms, in which one or more CH₂ groups are optionally replaced by —O—, —S—, —C(O)—, —C(S)—, —C(O)—O—, —O—C(O)—, —NR⁰—, —SiR⁰R⁰⁰—, —CF₂—, —CHR⁰═CR⁰⁰—, —CY¹═CY²— or —C≡C— in such a manner that O and/or S atoms are not linked directly to one another, and in which one or more H atoms are optionally replaced by F, Cl, Br, I or CN, or denote aryl, heteroaryl, aryloxy or heteroaryloxy with 4 to 20 ring atoms which is optionally substituted, preferably by halogen or by one or more of the aforementioned alkyl or cyclic alkyl groups,
Y¹ and Y² are independently of each other H, F, Cl or CN,
R⁰ and R⁰⁰ are independently of each other H or optionally substituted C₁₋₄₀ carbyl or hydrocarbyl, and preferably denote H or alkyl with 1 to 12 C-atoms.

The invention further relates to a formulation comprising one or more compounds comprising a unit of formula I and one or more solvents, preferably selected from organic solvents.

The invention further relates to an organic semiconducting formulation comprising one or more compounds comprising a unit of formula I, one or more organic binders, or precursors thereof, preferably having a permittivity ∈ at 1,000 Hz and 20° C. of 3.3 or less, and optionally one or more solvents.

The invention further relates to the use of units of formula I as electron donor units in semiconducting polymers.

The invention further relates to a conjugated polymer comprising one or more repeating units, wherein said repeating units contain a unit of formula I and/or one or more groups selected from aryl and heteroaryl groups that are optionally substituted, and wherein at least one repeating unit in the polymer contains at least one unit of formula I.

The invention further relates to monomers containing a unit of formula I and further containing one or more reactive groups which can be reacted to form a conjugated polymer as described above and below.

The invention further relates to a semiconducting polymer comprising one or more units of formula I as electron acceptor units, and preferably further comprising one or more units having electron donor properties.

The invention further relates to the use of the compounds according to the present invention as electron donor or p-type semiconductor.

The invention further relates to the use of the compounds according to the present invention as electron donor component in a semiconducting material, formulation, polymer blend, device or component of a device.

The invention further relates to a semiconducting material, formulation, polymer blend, device or component of a device comprising a compound according to the present invention as electron donor component, and preferably further comprising one or more compounds having electron acceptor properties.

The invention further relates to a mixture or polymer blend comprising one or more compounds according to the present invention and one or more additional compounds which are preferably selected from compounds having one or more of semiconducting, charge transport, hole or electron transport, hole or electron blocking, electrically conducting, photoconducting or light emitting properties.

The invention further relates to a mixture or polymer blend as described above and below, which comprises one or more compounds of the present invention and one or more n-type organic semiconductor compounds, preferably selected from fullerenes or substituted fullerenes.

The invention further relates to a formulation comprising one or more compounds, polymers, formulations, mixtures or polymer blends according to the present invention and optionally one or more solvents, preferably selected from organic solvents.

The invention further relates to the use of a compound, polymer, formulation, mixture or polymer blend of the present invention as charge transport, semiconducting, electrically conducting, photoconducting or light emitting material, or in an optical, electrooptical, electronic, electroluminescent or photoluminescent device, or in a component of such a device or in an assembly comprising such a device or component.

The invention further relates to a charge transport, semiconducting, electrically conducting, photoconducting or light emitting material comprising a compound, polymer, formulation, mixture or polymer blend according to the present invention.

The invention further relates to an optical, electrooptical, electronic, electroluminescent or photoluminescent device, or a component thereof, or an assembly comprising it, which comprises a compound, polymer, formulation, mixture or polymer blend, or comprises a charge transport, semiconducting, electrically conducting, photoconducting or light emitting material, according to the present invention.

The optical, electrooptical, electronic, electroluminescent and photoluminescent devices include, without limitation, organic field effect transistors (OFET), organic thin film transistors (OTFT), organic light emitting diodes (OLED), organic light emitting transistors (OLET), organic photovoltaic devices (OPV), organic photodetectors (OPD), organic solar cells, laser diodes, Schottky diodes, photoconductors and photodetectors.

The components of the above devices include, without limitation, charge injection layers, charge transport layers, interlayers, planarising layers, antistatic films, polymer electrolyte membranes (PEM), conducting substrates and conducting patterns.

The assemblies comprising such devices or components include, without limitation, integrated circuits (IC), radio frequency identification (RFID) tags or security markings or security devices containing them, flat panel displays or backlights thereof, electrophotographic devices, electrophotographic recording devices, organic memory devices, sensor devices, biosensors and biochips.

In addition the compounds, polymers, formulations, mixtures or polymer blends of the present invention can be used as electrode materials in batteries and in components or devices for detecting and discriminating DNA sequences.

### DETAILED DESCRIPTION

The compounds, monomers and polymers of the present invention are easy to synthesize and exhibit advantageous properties. The conjugated polymers of the present invention show good processability for the device manufacture process, high solubility in organic solvents, and are especially suitable for large scale production using solution processing methods. At the same time, the co-polymers derived from monomers of the present invention and electron donor monomers show low bandgaps, high charge carrier mobilities, high external quantum efficiencies in BHJ solar cells, good morphology when used in p/n-type blends e.g. with fullerenes, high oxidative stability, and a long lifetime in electronic devices, and are promising materials for organic electronic OE devices, especially for OPV devices with high power conversion efficiency.

The unit of formula I is especially suitable as (electron) acceptor unit in both n-type and p-type semiconducting compounds, polymers or copolymers, in particular copolymers containing both donor and acceptor units, and for the preparation of blends of p-type and n-type semiconductors which are useful for application in bulk heterojunction photovoltaic devices.

In addition, the compounds show the following advantageous properties:
i) Compared to prior art compounds like DPP, expansion of the ring system will lead to alternative solubility and morphology profile. Such a difference will have an impact on the OFET and/or OPV device fabrication process and performance.
ii) Solubility can be introduced into the polymer or compound by inclusion of functional group on R₁ and R₂ positions of the 1,5-disubstituted-1,5-dihydro-[1,5]naphthyridine-2,6-dione core.
iii) The units of 1,5-disubstituted-1,5-dihydro-[1,5]naphthyridine-2,6-dione are planar structures that enable strong pi-pi stacking in the solid state leading to improved charge transport properties in the form of higher charge carrier mobility.
iv) According to modelling, polymers based on the 1,5-disubstituted-1,5-dihydro-[1,5]naphthyridine-2,6-dione core have an increased HOMO level than the DPP (diketopyrrolopyrrole) equivalent material, resulting in an improved charge injection in OFETs.
v) Additional fine-tuning of the electronic energies (HOMO/LUMO levels) by either careful selection of Arₓ units on each side of 1,5-disubstituted-1,5-dihydro-[1,5]naphthyridine-2,6-dione core or co-polymerisation with appropriate co-monomer(s) should afford candidate materials for OFET and/or OPV applications.
vi) Alternatively, fine-tuning of the electronic energies (HOMO/LUMO levels) and solubility of the resulting polymer or compound is achieved by careful selection of different Arₓ units generating asymmetric repeat units (in the polymer backbone) or compounds.

The synthesis of the unit of formula I, its functional derivatives, compounds, homopolymers, and co-polymers can be achieved based on methods that are known to the skilled person and described in the literature, as will be further illustrated herein.

As used herein, the term “polymer” will be understood to mean a molecule of high relative molecular mass, the structure of which essentially comprises the multiple repetition of units derived, actually or conceptually, from molecules of low relative molecular mass (Pure Appl. Chem., 1996, 68, 2291). The term “oligomer” will be understood to mean a molecule of intermediate relative molecular mass, the structure of which essentially comprises a small plurality of units derived, actually or conceptually, from molecules of lower relative molecular mass (Pure Appl. Chem., 1996, 68, 2291). In a preferred meaning as used herein present invention a polymer will be understood to mean a compound having >1, i.e. at least 2 repeat units, preferably ≧5 repeat units, and an oligomer will be understood to mean a compound with >1 and <10, preferably <5, repeat units.

Further, as used herein, the term “polymer” will be understood to mean a molecule that encompasses a backbone (also referred to as “main chain”) of one or more distinct types of repeat units (the smallest constitutional unit of the molecule) and is inclusive of the commonly known terms “oligomer”, “copolymer”, “homopolymer” and the like. Further, it will be understood that the term polymer is inclusive of, in addition to the polymer itself, residues from initiators, catalysts and other elements attendant to the synthesis of such a polymer, where such residues are understood as not being covalently incorporated thereto. Further, such residues and other elements, while normally removed during post polymerization purification processes, are typically mixed or co-mingled with the polymer such that they generally remain with the polymer when it is transferred between vessels or between solvents or dispersion media.

As used herein, in a formula showing a polymer or a repeat unit, like for example a unit of formula I or a polymer of formula III or IV, or their subformulae, an asterisk (*) will be understood to mean a chemical linkage to an adjacent unit or to a terminal group in the polymer backbone. In a ring, like for example a benzene or thiophene ring, an asterisk (*) will be understood to mean a C atom that is fused to an adjacent ring.

As used herein, the terms “repeat unit”, “repeating unit” and “monomeric unit” are used interchangeably and will be understood to mean the constitutional repeating unit (CRU), which is the smallest constitutional unit the repetition of which constitutes a regular macromolecule, a regular oligomer molecule, a regular block or a regular chain (Pure Appl. Chem., 1996, 68, 2291). As further used herein, the term “unit” will be understood to mean a structural unit which can be a repeating unit on its own, or can together with other units form a constitutional repeating unit.

As used herein, a “terminal group” will be understood to mean a group that terminates a polymer backbone. The expression “in terminal position in the backbone” will be understood to mean a divalent unit or repeat unit that is linked at one side to such a terminal group and at the other side to another repeat unit. Such terminal groups include endcap groups, or reactive groups that are attached to a monomer forming the polymer backbone which did not participate in the polymerisation reaction, like for example a group having the meaning of R⁵ or R⁶ as defined below.

As used herein, the term “endcap group” will be understood to mean a group that is attached to, or replacing, a terminal group of the polymer backbone. The endcap group can be introduced into the polymer by an endcapping process. Endcapping can be carried out for example by reacting the terminal groups of the polymer backbone with a monofunctional compound (“endcapper”) like for example an alkyl- or arylhalide, an alkyl- or arylstannane or an alkyl- or arylboronate. The endcapper can be added for example after the polymerisation reaction. Alternatively the endcapper can be added in situ to the reaction mixture before or during the polymerisation reaction. In situ addition of an endcapper can also be used to terminate the polymerisation reaction and thus control the molecular weight of the forming polymer. Typical endcap groups are for example H, phenyl and lower alkyl.

As used herein, the term “small molecule” will be understood to mean a monomeric compound which typically does not contain a reactive group by which it can be reacted to form a polymer, and which is designated to be used in monomeric form. In contrast thereto, the term “monomer” unless stated otherwise will be understood to mean a monomeric compound that carries one or more reactive functional groups by which it can be reacted to form a polymer.

As used herein, the terms “donor” or “donating” and “acceptor” or “accepting” will be understood to mean an electron donor or electron acceptor, respectively. “Electron donor” will be understood to mean a chemical entity that donates electrons to another compound or another group of atoms of a compound. “Electron acceptor” will be understood to mean a chemical entity that accepts electrons transferred to it from another compound or another group of atoms of a compound. (see also U.S. Environmental Protection Agency, 2009, Glossary of technical terms, http://www.epa.gov/oust/cat/TUMGLOSS.HTM, or International Union or Pure and Applied Chemistry, Compendium of Chemical Terminology, Gold Book).

As used herein, the term “n-type” or “n-type semiconductor” will be understood to mean an extrinsic semiconductor in which the conduction electron density is in excess of the mobile hole density, and the term “p-type” or “p-type semiconductor” will be understood to mean an extrinsic semiconductor in which mobile hole density is in excess of the conduction electron density (see also, J. Thewlis, Concise Dictionary of Physics, Pergamon Press, Oxford, 1973).

As used herein, the term “leaving group” will be understood to mean an atom or group (which may be charged or uncharged) that becomes detached from an atom in what is considered to be the residual or main part of the molecule taking part in a specified reaction (see also Pure Appl. Chem., 1994, 66, 1134).

As used herein, the term “conjugated” will be understood to mean a compound (for example a polymer) that contains mainly C atoms with sp²-hybridisation (or optionally also sp-hybridisation), and wherein these C atoms may also be replaced by hetero atoms. In the simplest case this is for example a compound with alternating C—C single and double (or triple) bonds, but is also inclusive of compounds with aromatic units like for example 1,4-phenylene. The term “mainly” in this connection will be understood to mean that a compound with naturally (spontaneously) occurring defects, which may lead to interruption of the conjugation, is still regarded as a conjugated compound.

As used herein, unless stated otherwise the molecular weight is given as the number average molecular weight Mₙ or weight average molecular weight M_{w}, which is determined by gel permeation chromatography (GPC) against polystyrene standards in eluent solvents such as tetrahydrofuran, trichloromethane (TCM, chloroform), chlorobenzene or 1,2,4-trichloro-benzene. Unless stated otherwise, 1,2,4-trichlorobenzene is used as solvent. The degree of polymerization, also referred to as total number of repeat units, n, will be understood to mean the number average degree of polymerization given as n=Mₙ/M_{U}, wherein Mₙ is the number average molecular weight and M_{U} is the molecular weight of the single repeat unit, see J. M. G. Cowie, Polymers: Chemistry & Physics of Modern Materials, Blackie, Glasgow, 1991.

As used herein, the term “carbyl group” will be understood to mean denotes any monovalent or multivalent organic radical moiety which comprises at least one carbon atom either without any non-carbon atoms (like for example —C≡C—), or optionally combined with at least one non-carbon atom such as N, O, S, P, Si, Se, As, Te or Ge (for example carbonyl etc.). The term “hydrocarbyl group” will be understood to mean a carbyl group that does additionally contain one or more H atoms and optionally contains one or more hetero atoms like for example N, O, S, P, Si, Se, As, Te or Ge.

As used herein, the term “hetero atom” will be understood to mean an atom in an organic compound that is not a H- or C-atom, and preferably will be understood to mean N, O, S, P, Si, Se, As, Te or Ge.

A carbyl or hydrocarbyl group comprising a chain of 3 or more C atoms may be straight-chain, branched and/or cyclic, including spiro and/or fused rings.

Preferred carbyl and hydrocarbyl groups include alkyl, alkoxy, alkylcarbonyl, alkoxycarbonyl, alkylcarbonyloxy and alkoxycarbonyloxy, each of which is optionally substituted and has 1 to 40, preferably 1 to 25, very preferably 1 to 18 C atoms, furthermore optionally substituted aryl or aryloxy having 6 to 40, preferably 6 to 25 C atoms, furthermore alkylaryloxy, arylcarbonyl, aryloxycarbonyl, arylcarbonyloxy and aryloxycarbonyloxy, each of which is optionally substituted and has 6 to 40, preferably 7 to 40 C atoms, wherein all these groups do optionally contain one or more hetero atoms, preferably selected from N, O, S, P, Si, Se, As, Te and Ge.

The carbyl or hydrocarbyl group may be a saturated or unsaturated acyclic group, or a saturated or unsaturated cyclic group. Unsaturated acyclic or cyclic groups are preferred, especially aryl, alkenyl and alkynyl groups (especially ethynyl). Where the C₁-C₄₀ carbyl or hydrocarbyl group is acyclic, the group may be straight-chain or branched. The C₁-C₄₀ carbyl or hydrocarbyl group includes for example: a C₁-C₄₀ alkyl group, a C₁-C₄₀ fluoroalkyl group, a C₁-C₄₀ alkoxy or oxaalkyl group, a C₂-C₄₀ alkenyl group, a C₂-C₄₀ alkynyl group, a C₃-C₄₀ allyl group, a C₄-C₄₀ alkyldienyl group, a C₄-C₄₀ polyenyl group, a C₂-C₄₀ ketone group, a C₂-C₄₀ ester group, a C₆-C₁₈ aryl group, a C₆-C₄₀ alkylaryl group, a C₆-C₄₀ arylalkyl group, a C₄-C₄₀ cycloalkyl group, a C₄-C₄₀ cycloalkenyl group, and the like. Preferred among the foregoing groups are a C₁-C₂₀ alkyl group, a C₁-C₂₀ fluoroalkyl group, a C₂-C₂₀ alkenyl group, a C₂-C₂₀ alkynyl group, a C₃-C₂₀ allyl group, a C₄-C₂₀ alkyldienyl group, a C₂-C₂₀ ketone group, a C₂-C₂₀ ester group, a C₆-C₁₂ aryl group, and a C₄-C₂₀ polyenyl group, respectively. Also included are combinations of groups having carbon atoms and groups having hetero atoms, like e.g. an alkynyl group, preferably ethynyl, that is substituted with a silyl group, preferably a trialkylsilyl group.

The terms “aryl” and “heteroaryl” as used herein preferably mean a mono-, bi- or tricyclic aromatic or heteroaromatic group with 4 to 30 ring C atoms that may also comprise condensed rings and is optionally substituted with one or more groups L,

wherein L is selected from halogen, —CN, —NC, —NCO, —NCS, —OCN, —SCN, —C(═O)NR⁰R⁰⁰, —C(═O)X⁰, —C(═O)R⁰, —NH₂, —NR⁰R⁰⁰, —SH, —SR⁰, —SO₃H, —SO₂R⁰, —OH, —NO₂, —CF₃, —SF₅, P-Sp-, optionally substituted silyl, or carbyl or hydrocarbyl with 1 to 40 C atoms that is optionally substituted and optionally comprises one or more hetero atoms, and is preferably alkyl, alkoxy, thiaalkyl, alkylcarbonyl, alkoxycarbonyl or alkoxycarbonyloxy with 1 to 20 C atoms that is optionally fluorinated, and R⁰, R⁰⁰, X⁰, P and Sp have the meanings given above and below.

Very preferred substituents L are selected from halogen, most preferably F, or alkyl, alkoxy, oxaalkyl, thioalkyl, fluoroalkyl and fluoroalkoxy with 1 to 12 C atoms or alkenyl, alkynyl with 2 to 12 C atoms.

Especially preferred aryl and heteroaryl groups are phenyl in which, in addition, one or more CH groups may be replaced by N, naphthalene, thiophene, selenophene, thienothiophene, dithienothiophene, fluorene and oxazole, all of which can be unsubstituted, mono- or polysubstituted with L as defined above. Very preferred rings are selected from pyrrole, preferably N-pyrrole, furan, pyridine, preferably 2- or 3-pyridine, pyrimidine, pyridazine, pyrazine, triazole, tetrazole, pyrazole, imidazole, isothiazole, thiazole, thiadiazole, isoxazole, oxazole, oxadiazole, thiophene, preferably 2-thiophene, selenophene, preferably 2-selenophene, thieno[3,2-b]thiophene, thieno[2,3-b]thiophene, furo[3,2-b]furan, furo[2,3-b]furan, seleno[3,2-b]selenophene, seleno[2,3-b]selenophene, thieno[3,2-b]selenophene, thieno[3,2-b]furan, indole, isoindole, benzo[b]furan, benzo[b]thiophene, benzo[1,2-b;4,5-b′]dithiophene, benzo[2,1-b;3,4-b′]dithiophene, quinole, 2-methylquinole, isoquinole, quinoxaline, quinazoline, benzotriazole, benzimidazole, benzothiazole, benzisothiazole, benzisoxazole, benzoxadiazole, benzoxazole, benzothiadiazole, all of which can be unsubstituted, mono- or polysubstituted with L as defined above. Further examples of aryl and heteroaryl groups are those selected from the groups shown hereinafter.

An alkyl or alkoxy radical, i.e. where the terminal CH₂ group is replaced by —O—, can be straight-chain or branched. It is preferably straight-chain, has 2, 3, 4, 5, 6, 7 or 8 carbon atoms and accordingly is preferably ethyl, propyl, butyl, pentyl, hexyl, heptyl, octyl, ethoxy, propoxy, butoxy, pentoxy, hexoxy, heptoxy, or octoxy, furthermore methyl, nonyl, decyl, undecyl, dodecyl, tridecyl, tetradecyl, pentadecyl, nonoxy, decoxy, undecoxy, dodecoxy, tridecoxy or tetradecoxy, for example.

An alkenyl group, wherein one or more CH₂ groups are replaced by —CH═CH— can be straight-chain or branched. It is preferably straight-chain, has 2 to 10 C atoms and accordingly is preferably vinyl, prop-1-, or prop-2-enyl, but-1-, 2- or but-3-enyl, pent-1-, 2-, 3- or pent-4-enyl, hex-1-, 2-, 3-, 4- or hex-5-enyl, hept-1-, 2-, 3-, 4-, 5- or hept-6-enyl, oct-1-, 2-, 3-, 4-, 5-, 6- or oct-7-enyl, non-1-, 2-, 3-, 4-, 5-, 6-, 7- or non-8-enyl, dec-1-, 2-, 3-, 4-, 5-, 6-, 7-, 8- or dec-9-enyl.

Especially preferred alkenyl groups are C₂-C₇-1E-alkenyl, C₄-C₇-3E-alkenyl, C₅-C₇-4-alkenyl, C₆-C₇-5-alkenyl and C₇-6-alkenyl, in particular C₂-C₇-1E-alkenyl, C₄-C₇-3E-alkenyl and C₅-C₇-4-alkenyl. Examples for particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Groups having up to 5 C atoms are generally preferred.

An oxaalkyl group, i.e. where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example oxaalkyl, i.e. where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example.

In an alkyl group wherein one CH₂ group is replaced by —O— and one by —C(O)—, these radicals are preferably neighboured. Accordingly these radicals together form a carbonyloxy group —C(O)—O— or an oxycarbonyl group —O—C(O)—. Preferably this group is straight-chain and has 2 to 6 C atoms. It is accordingly preferably acetyloxy, propionyloxy, butyryloxy, pentanoyloxy, hexanoyloxy, acetyloxymethyl, propionyloxymethyl, butyryloxymethyl, pentanoyloxymethyl, 2-acetyloxyethyl, 2-propionyloxy-ethyl, 2-butyryloxyethyl, 3-acetyloxypropyl, 3-propionyloxypropyl, 4-acetyloxybutyl, methoxycarbonyl, ethoxycarbonyl, propoxycarbonyl, butoxycarbonyl, pentoxycarbonyl, methoxycarbonylmethyl, ethoxy-carbonylmethyl, propoxycarbonylmethyl, butoxycarbonylmethyl, 2-(methoxycarbonyl)ethyl, 2-(ethoxycarbonyl)ethyl, 2-(propoxy-carbonyl)ethyl, 3-(methoxycarbonyl)propyl, 3-(ethoxycarbonyl)propyl, 4-(methoxycarbonyl)-butyl.

An alkyl group wherein two or more CH₂ groups are replaced by —O— and/or —C(O)O— can be straight-chain or branched. It is preferably straight-chain and has 3 to 12 C atoms. Accordingly it is preferably bis-carboxy-methyl, 2,2-bis-carboxy-ethyl, 3,3-bis-carboxy-propyl, 4,4-bis-carboxy-butyl, 5,5-bis-carboxy-pentyl, 6,6-bis-carboxy-hexyl, 7,7-bis-carboxy-heptyl, 8,8-bis-carboxy-octyl, 9,9-bis-carboxy-nonyl, 10,10-bis-carboxy-decyl, bis-(methoxycarbonyl)-methyl, 2,2-bis-(methoxycarbonyl)-ethyl, 3,3-bis-(methoxycarbonyl)-propyl, 4,4-bis-(methoxycarbonyl)-butyl, 5,5-bis-(methoxycarbonyl)-pentyl, 6,6-bis-(methoxycarbonyl)-hexyl, 7,7-bis-(methoxycarbonyl)-heptyl, 8,8-bis-(methoxycarbonyl)-octyl, bis-(ethoxycarbonyl)-methyl, 2,2-bis-(ethoxycarbonyl)-ethyl, 3,3-bis-(ethoxycarbonyl)-propyl, 4,4-bis-(ethoxycarbonyl)-butyl, 5,5-bis-(ethoxycarbonyl)-hexyl.

A thioalkyl group, i.e. where one CH₂ group is replaced by —S—, is preferably straight-chain thiomethyl (—SCH₃), 1-thioethyl (—SCH₂CH₃), 1-thiopropyl (=—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH₂ group adjacent to the sp² hybridised vinyl carbon atom is replaced.

A fluoroalkyl group is preferably perfluoroalkyl CᵢF₂ᵢ₊₁, wherein i is an integer from 1 to 15, in particular CF₃, C₂F₅, C₃F₇, C₄F₉, C₅F₁₁, C₆F₁₃, C₇F₁₅ or C₈F₁₇, very preferably C₆F₁₃, or partially fluorinated alkyl, in particular 1,1-difluoroalkyl, all of which are straight-chain or branched.

Alkyl, alkoxy, alkenyl, oxaalkyl, thioalkyl, carbonyl and carbonyloxy groups can be achiral or chiral groups. Particularly preferred chiral groups are 2-butyl (=1-methylpropyl), 2-methylbutyl, 2-methylpentyl, 3-methylpentyl, 2-ethylhexyl, 2-propylpentyl, in particular 2-methylbutyl, 2-methylbutoxy, 2-methylpentoxy, 3-methylpentoxy, 2-ethyl-hexoxy, 1-methylhexoxy, 2-octyloxy, 2-oxa-3-methylbutyl, 3-oxa-4-methyl-pentyl, 4-methylhexyl, 2-hexyl, 2-octyl, 2-nonyl, 2-decyl, 2-dodecyl, 6-meth-oxyoctoxy, 6-methyloctoxy, 6-methyloctanoyloxy, 5-methylheptyloxy-carbonyl, 2-methylbutyryloxy, 3-methylvaleroyloxy, 4-methylhexanoyloxy, 2-chloropropionyloxy, 2-chloro-3-methylbutyryloxy, 2-chloro-4-methyl-valeryl-oxy, 2-chloro-3-methylvaleryloxy, 2-methyl-3-oxapentyl, 2-methyl-3-oxa-hexyl, 1-methoxypropyl-2-oxy, 1-ethoxypropyl-2-oxy, 1-propoxypropyl-2-oxy, 1-butoxypropyl-2-oxy, 2-fluorooctyloxy, 2-fluorodecyloxy, 1,1,1-trifluoro-2-octyloxy, 1,1,1-trifluoro-2-octyl, 2-fluoromethyloctyloxy for example. Very preferred are 2-hexyl, 2-octyl, 2-octyloxy, 1,1,1-trifluoro-2-hexyl, 1,1,1-trifluoro-2-octyl and 1,1,1-trifluoro-2-octyloxy.

Preferred achiral branched groups are isopropyl, isobutyl (=methylpropyl), isopentyl (=3-methylbutyl), tertbutyl, isopropoxy, 2-methyl-propoxy and 3-methylbutoxy.

In a preferred embodiment, R¹ and R² are independently of each other selected from primary, secondary or tertiary alkyl or alkoxy with 1 to 30 C atoms, wherein one or more H atoms are optionally replaced by F, or aryl, aryloxy, heteroaryl or heteroaryloxy that is optionally alkylated or alkoxylated and has 4 to 30 ring atoms. Very preferred groups of this type are selected from the group consisting of the following formulae

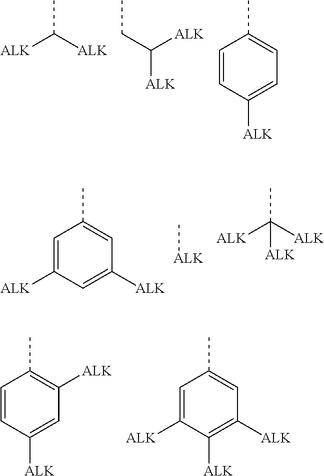

wherein “ALK” denotes optionally fluorinated, preferably linear, alkyl or alkoxy with 1 to 20, preferably 1 to 12 C-atoms, in case of tertiary groups very preferably 1 to 9 C atoms, and the dashed line denotes the link to the ring to which these groups are attached. Especially preferred among these groups are those wherein all ALK subgroups are identical.

—CY¹¹═CY¹²— is preferably —CH═CH—, —CF═CF— or —CH═C(CN)—.

As used herein, “halogen” includes F, Cl, Br or I, preferably F, Cl or Br.

A used herein, —CO—, —C(═O)— and —C(O)— will be understood to mean a carbonyl group, i.e. a group having the structure

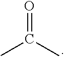

In the units of formula I and its preferred subformulae, R¹ and R² preferably denote straight-chain, branched or cyclic alkyl with 1 to 30 C atoms which is unsubstituted or substituted by one or more F atoms.

Further preferably one of R¹ and R² is H and the other is different from H, and is preferably straight-chain, branched or cyclic alkyl with 1 to 30 C atoms which is unsubstituted or substituted by one or more F atoms.

Further preferably R¹ and/or R² are independently of each other selected from the group consisting of aryl and heteroaryl, each of which is optionally fluorinated, alkylated or alkoxylated and has 4 to 30 ring atoms.

If R¹ and/or R² in formula I denote substituted aryl or heteroaryl, it is preferably substituted by one or more groups L, wherein L is selected from P-Sp-, F, Cl, Br, I, —OH, —CN, —NO₂, —NCO, —NCS, —OCN, —SCN, —C(═O)NR⁰R⁰⁰, —C(═O)X⁰, —C(═O)R⁰, —NR⁰R⁰⁰, C(═O)OH, optionally substituted aryl or heteroaryl having 4 to 20 ring atoms, or straight chain, branched or cyclic alkyl with 1 to 20, preferably 1 to 12 C atoms wherein one or more non-adjacent CH₂ groups are optionally replaced, in each case independently from one another, by —O—, —S—, —NR⁰—, —SiR⁰R⁰⁰—, —C(═O)—, —C(═O)O—, —CY¹═CY²— or —C≡C— in such a manner that O and/or S atoms are not linked directly to one another and which is unsubstituted or substituted with one or more F or Cl atoms or OH groups, X⁰ is halogen, preferably F, Cl or Br, and Y¹, Y², R⁰ and R⁰⁰ have the meanings given above and below.

Further preferably R¹ and/or R² in formula I denote aryl or heteroaryl that is substituted by one or more straight-chain, branched or cyclic alkyl groups with 1 to 30 C atoms, in which one or more non-adjacent CH₂ groups are optionally replaced by one or more non-adjacent CH₂ groups are optionally replaced by —O—, —S—, —C(O)—, —C(O)—O—, —O—C(O)—, —O—C(O)—O—, —NR⁰—, —SiR⁰R⁰⁰—, —CF₂—, —CHR⁰═CR⁰⁰—, —CY¹═CY²— or —C≡C— in such a manner that O and/or S atoms are not linked directly to one another, and in which one or more H atoms are optionally replaced by F, Cl, Br, I or CN.

The compounds according to the present invention include small molecules, monomers, oligomers and polymers.

Oligomers and polymers according to the present invention preferably comprise one or more units of formula I as defined above and below.

Preferred polymers according to the present invention comprise one or more repeating units of formula IIa or IIb:

—[(Ar¹)ₐ—(U)_{b}-(Ar²)_{c}—(Ar³)_{d}]— IIa

—[(U)_{b}-(Ar¹)ₐ—(U)_{b}-(Ar²)_{c}—(Ar³)_{d}]— IIb

wherein
U is a unit of formula I,
Ar¹, Ar², Ar³ are, on each occurrence identically or differently, and independently of each other, aryl or heteroaryl that is different from U, preferably has 5 to 30 ring atoms, and is optionally substituted, preferably by one or more groups R^{S},
R^{S} is on each occurrence identically or differently F, Br, Cl, —CN, —NC, —NCO, —NCS, —OCN, —SCN, —C(O)NR⁰R⁰⁰, —C(O)X⁰, —C(O)R⁰, —C(O)OR⁰, —NH₂, —NR⁰R⁰⁰, —SH, —SR⁰, —SO₃H, —SO₂R⁰, —OH, —NO₂, —CF₃, —SF₅, optionally substituted silyl, carbyl or hydrocarbyl with 1 to 40 C atoms that is optionally substituted and optionally comprises one or more hetero atoms,
R⁰ and R⁰⁰ are independently of each other H or optionally substituted C₁₋₄₀ carbyl or hydrocarbyl, and preferably denote H or alkyl with 1 to 12 C-atoms,

X⁰ is halogen, preferably F, Cl or Br,
a, b, c are on each occurrence identically or differently 0, 1 or 2,
d is on each occurrence identically or differently 0 or an integer from 1 to 10,

wherein the polymer comprises at least one repeating unit of formula IIa or IIb wherein b is at least 1.

Further preferred polymers according to the present invention comprise, in addition to the units of formula I, IIa or IIb, one or more repeating units selected from monocyclic or polycyclic aryl or heteroaryl groups that are optionally substituted.

These additional repeating units are preferably selected of formula IIIa and IIIb

—[(Ar¹)ₐ-(D)_{b}-(Ar²)_{c}—(Ar³)_{d}]— IIIa

-[(D)_{b}-(Ar¹)ₐ-(D)_{b}-(Ar²)_{c}—(Ar³)_{d}]— IIIb

wherein Ar¹, Ar², Ar³, a, b, c and d are as defined in formula IIa, and D is an aryl or heteroaryl group that is different from U and Ar¹⁻³, preferably has 5 to 30 ring atoms, is optionally substituted by one or more groups R^{S} as defined above and below, and is preferably selected from aryl or heteroaryl groups having electron donor properties, wherein the polymer comprises at least one repeating unit of formula IIIa or IIIb wherein b is at least 1.

R^{S} preferably has one of the meanings given for R¹.

The conjugated polymers according to the present invention are preferably selected of formula IV:

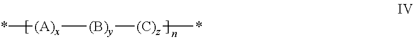

wherein
A, B, C independently of each other denote a distinct unit of formula I, IIa, IIb, IIIa, IIIb, or their preferred subformulae,
x is >0 and ≦1,
y is ≧0 and <1,
z is ≧0 and <1,
x+y+z is 1, and
n is an integer >1.

Preferred polymers of formula IV are selected of the following formulae

*—[(Ar¹—U—Ar²)ₓ—(Ar³)_{y}]ₙ—* IVa

*—[(Ar¹—U—Ar²)ₓ—(Ar³—Ar³)_{y}]ₙ—* IVb

*—[(Ar¹—U—Ar²)ₓ—(Ar³—Ar³—Ar³)_{y}]ₙ—* IVc

*—[(Ar¹)ₐ—(U)_{b}-(Ar²)_{c}—(Ar³)_{d}]ₙ-* IVd

*—([(Ar¹)ₐ—(U)_{b}-(Ar²)_{c}—(Ar³)_{d}]ₓ—[(Ar¹)ₐ-(D)_{b}-(Ar²)_{c}—(Ar³)_{d}]_{y})ₙ—* IVe

*—[(U—Ar¹—U)ₓ—(Ar²—Ar³)_{y}]ₙ—* IVf

*—[(U—Ar¹—U)ₓ—(Ar²—Ar³—Ar²)_{y}]ₙ—* IVg

*—[(U)_{b}-(Ar¹)ₐ—(U)_{b}-(Ar²)_{c}]ₙ—* IVh

*—([(U)_{b}-(Ar¹)ₐ—(U)_{b}-(Ar²)_{c}]ₓ-[(D)_{b}-(Ar¹)ₐ-(D)_{b}-(Ar²)_{d}]_{y})ₙ—* IVi

*—[(U—Ar¹)ₓ—(U—Ar²)_{y}—(U—Ar³)_{z}]ₙ-* IVk

wherein U, Ar¹, Ar², Ar³, a, b, c and d have in each occurrence identically or differently one of the meanings given in formula IIa, D has on each occurrence identically or differently one of the meanings given in formula IIIa, and x, y, z and n are as defined in formula IV, wherein these polymers can be alternating or random copolymers, and wherein in formula IVd and IVe in at least one of the repeating units [(Ar¹)ₐ—(U)_{b}-(Ar²)_{c}—(Ar³)_{d}] and in at least one of the repeating units [(Ar¹)ₐ-(D)_{b}-(Ar²)_{c}—(Ar³)_{d}] b is at least 1 and wherein in formula IVh and IVi in at least one of the repeating units [(U)_{b}-(Ar¹)ₐ—(U)_{b}-(Ar²)_{d}] and in at least one of the repeating units [(U)_{b}-(Ar¹)ₐ—(U)_{b}-(Ar²)_{d}] b is at least 1.

In the polymers according to the present invention, the total number of repeating units n is preferably from 2 to 10,000. The total number of repeating units n is preferably ≧5, very preferably ≧10, most preferably ≧50, and preferably ≦500, very preferably ≦1,000, most preferably ≦2,000, including any combination of the aforementioned lower and upper limits of n.

The polymers of the present invention include homopolymers and copolymers, like statistical or random copolymers, alternating copolymers and block copolymers, as well as combinations thereof.

Especially preferred are polymers selected from the following groups:
Group A consisting of homopolymers of the unit U or (Ar¹—U) or (Ar¹—U—Ar²) or (Ar¹—U—Ar³) or (U—Ar²—Ar³) or (Ar¹—U—Ar²—Ar³) or (U—Ar¹—U), i.e. where all repeating units are identical,
Group B consisting of random or alternating copolymers formed by identical units (Ar¹—U—Ar²) or (U—Ar¹—U) and identical units (Ar³),
Group C consisting of random or alternating copolymers formed by identical units (Ar¹—U—Ar²) or (U—Ar¹—U) and identical units (A¹),
Group D consisting of random or alternating copolymers formed by identical units (Ar¹—U—Ar²) or (U—Ar¹—U) and identical units (Ar¹-D-Ar²) or (D-Ar¹-D),

wherein in all these groups U, D, Ar¹, Ar² and Ar³ are as defined above and below, in groups A, B and C Ar¹, Ar² and Ar³ are different from a single bond, and in group D one of Ar¹ and Ar² may also denote a single bond.

Preferred polymers of formula IV and IVa to IVe are selected of formula V

R⁵-chain-R⁶ V

wherein “chain” denotes a polymer chain of formulae IV or IVa to IVk, and R⁵ and R⁶ have independently of each other one of the meanings of R¹ as defined above, or denote, independently of each other, H, F, Br, Cl, I, —CH₂Cl, —CHO, —CR′═CR″₂, —SiR′R″R′″, —SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′ or an endcap group, X′ and X″ denote halogen, R′, R″ and R′″ have independently of each other one of the meanings of R⁰ given in formula I, and two of R′, R″ and R′″ may also form a ring together with the hetero atom to which they are attached.

Preferred endcap groups R⁵ and R⁶ are H, C₁₋₂₀ alkyl, or optionally substituted C₆₋₁₂ aryl or C₂₋₁₀ heteroaryl, very preferably H or phenyl.

In the polymers represented by formula IV, IVa to IVk and V, x, y and z denote the mole fraction of units A, B and C, respectively, and n denotes the degree of polymerisation or total number of units A, B and C. These formulae includes block copolymers, random or statistical copolymers and alternating copolymers of A, B and C, as well as homopolymers of A for the case when x>0 and y=z=0.

The invention further relates to monomers of formula VIa and VIb

R⁷—(Ar¹)ₐ—U—(Ar²)_{c}—R⁸ VIa

R⁷—U—(Ar¹)ₐ—U—R⁸ VIb

wherein U, Ar¹, Ar², a and b have the meanings of formula IIa, or one of the preferred meanings as described above and below, and R⁷ and R⁸ are, preferably independently of each other, selected from the group consisting of Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ and —Sn(Z⁴)₃, wherein X⁰ is halogen, preferably Cl, Br or I, Z¹⁻⁴ are selected from the group consisting of alkyl and aryl, each being optionally substituted, and two groups Z² may also together form a cyclic group.

Especially preferred are monomers of the following formulae

R⁷—Ar¹—U—Ar²—R⁸ VI1

R⁷—U—R⁸ VI2

R⁷—Ar¹—U—R⁸ VI3

R⁷—U—Ar²—R⁸ VI4

R⁷—U—Ar¹—U—R⁸ VI5

wherein U, Ar¹, Ar², R⁷ and R⁸ are as defined in formula VI.

Especially preferred are repeating units, monomers and polymers of formulae I, IIa, IIb, IIIa. IIIb, IV, IVa-IVk, V, VIa, VIb and their subformulae wherein one or more of D, Ar¹, Ar² and Ar³ denote aryl or heteroaryl, preferably having electron donor properties, selected from the group consisting of the following formulae

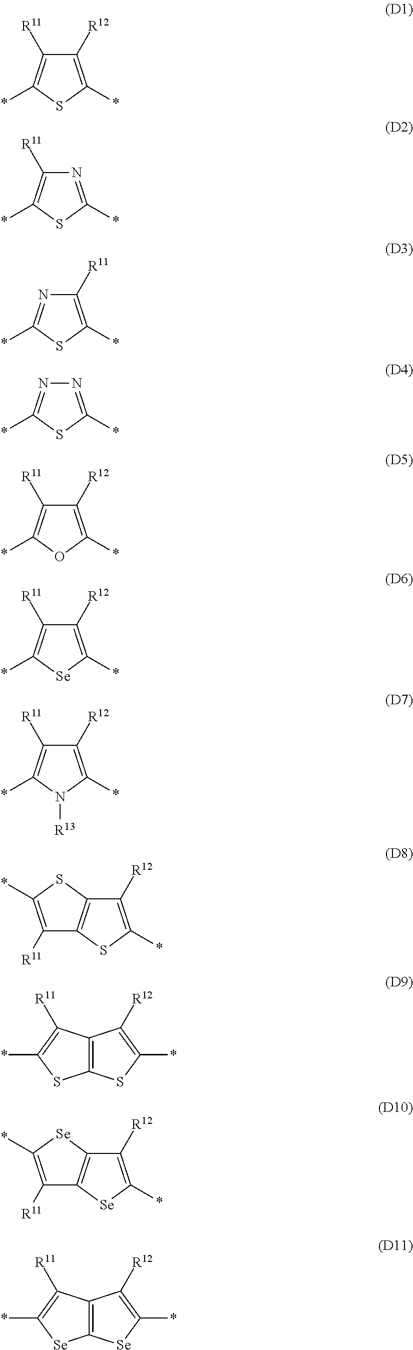

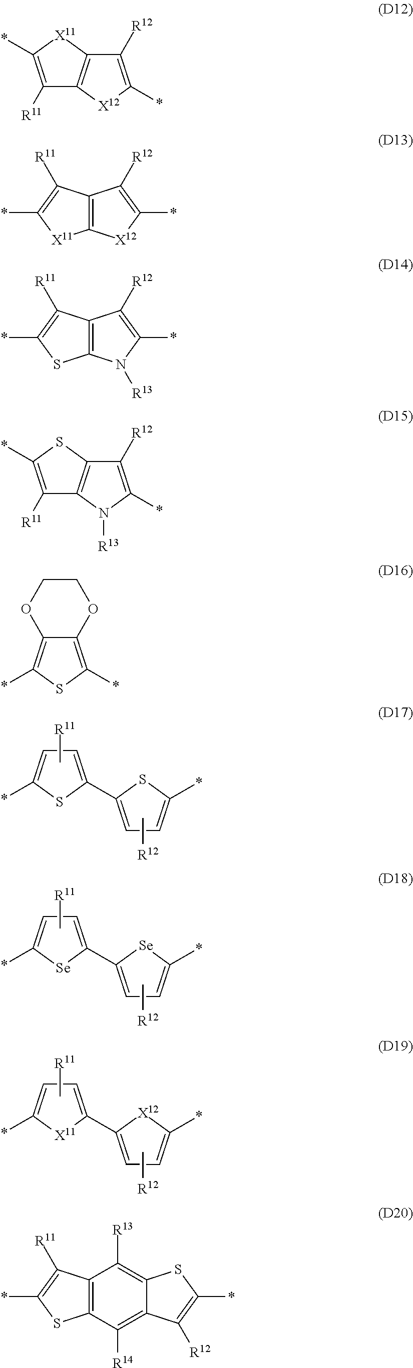

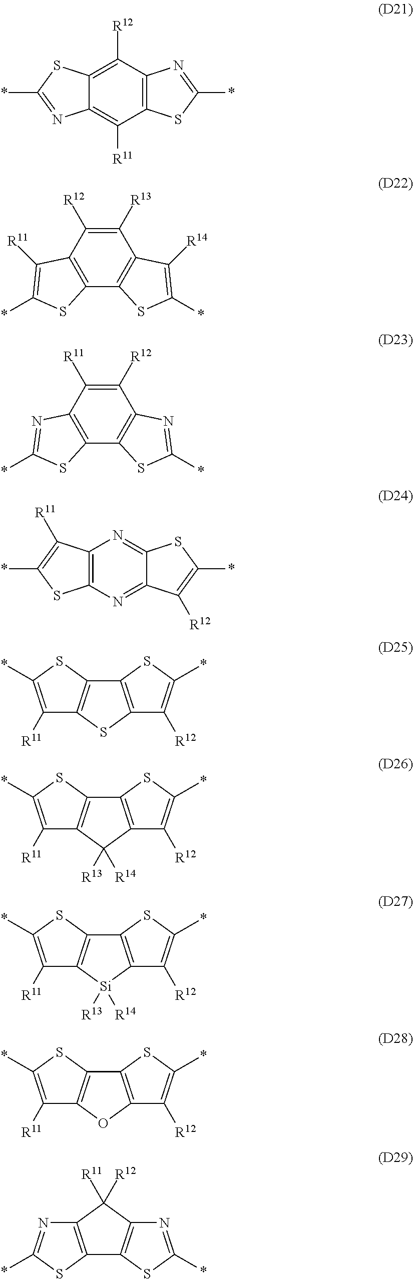

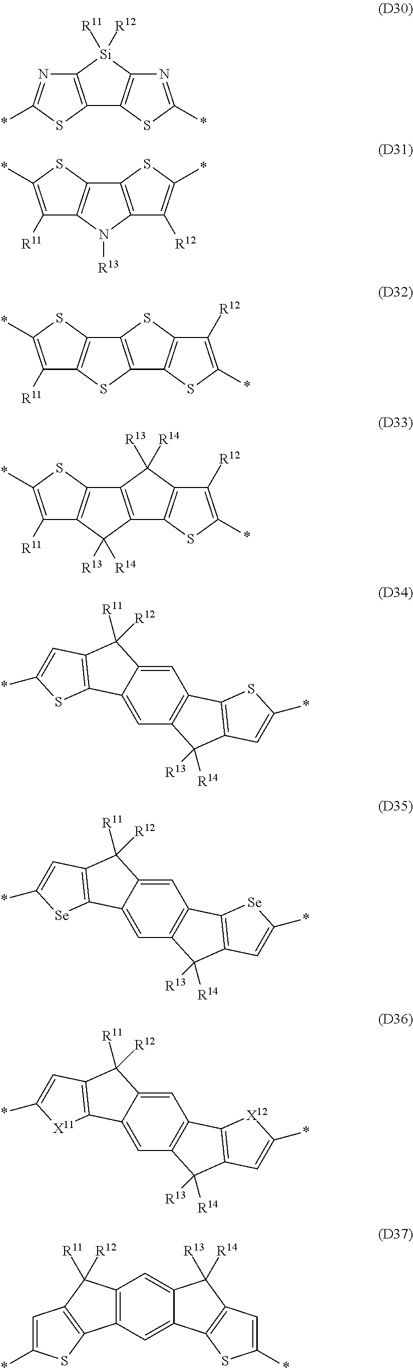

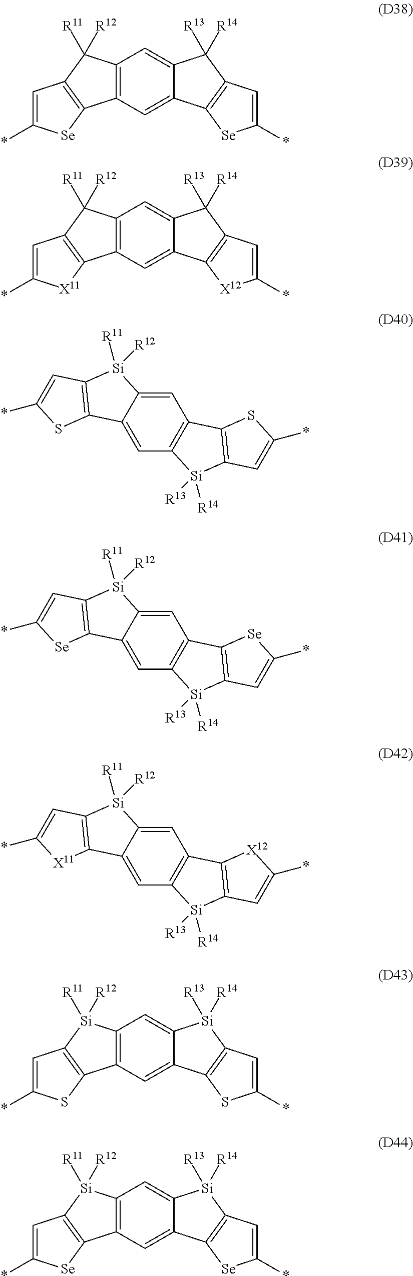

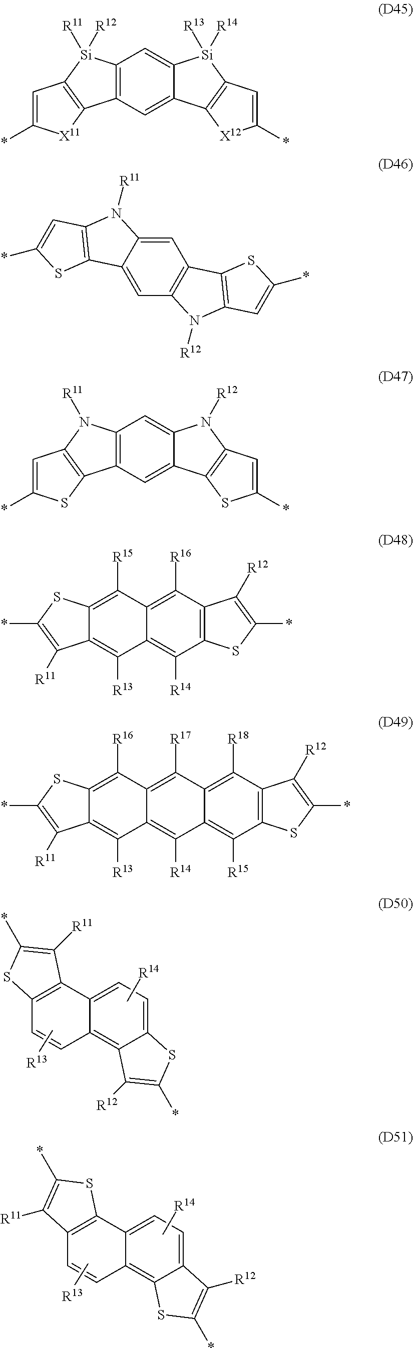

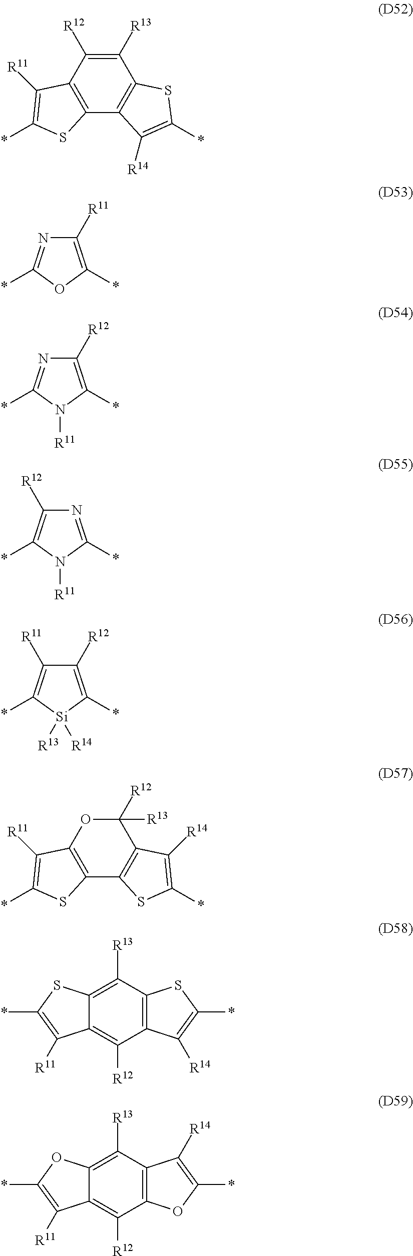

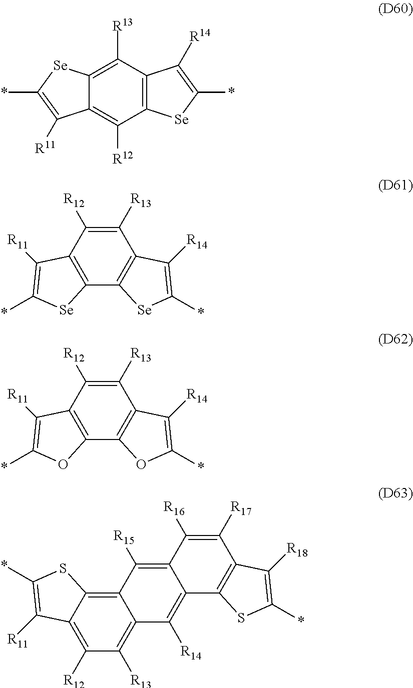

wherein one of X¹¹ and X¹² is S and the other is Se, and R¹¹, R¹², R¹³, R¹⁴, R¹⁵, R¹⁶, R¹⁷ and R¹⁸ independently of each other denote H or have one of the meanings of R¹ as defined above and below.

Further preferred are repeating units, monomers and polymers of formulae I, IIa, IIb, IIIa. IIIb, IV, IVa-IVk, V, VIa, VIb and their subformulae wherein Ar³ denotes aryl or heteroaryl, preferably having electron acceptor properties, selected from the group consisting of the following formulae

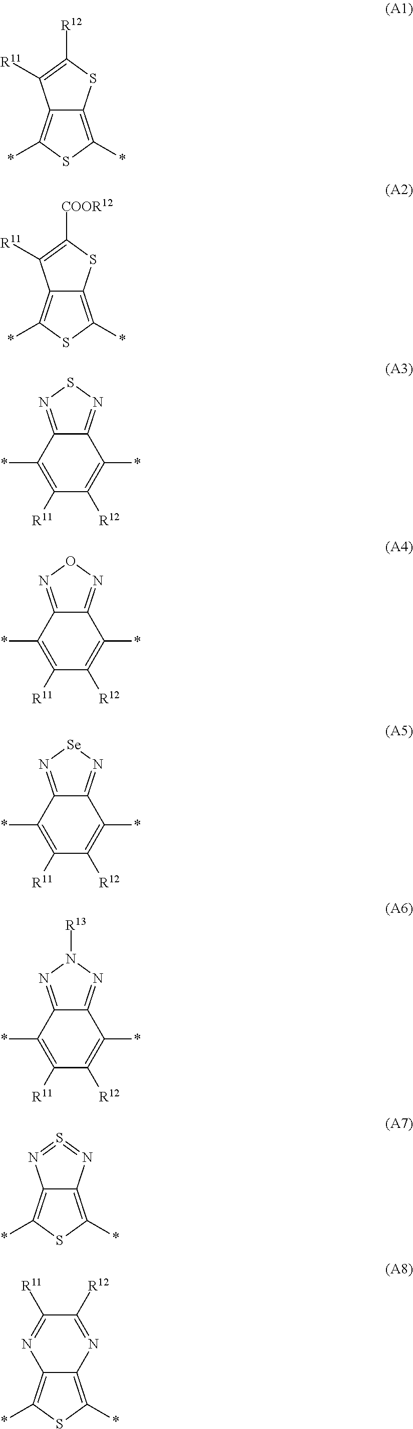

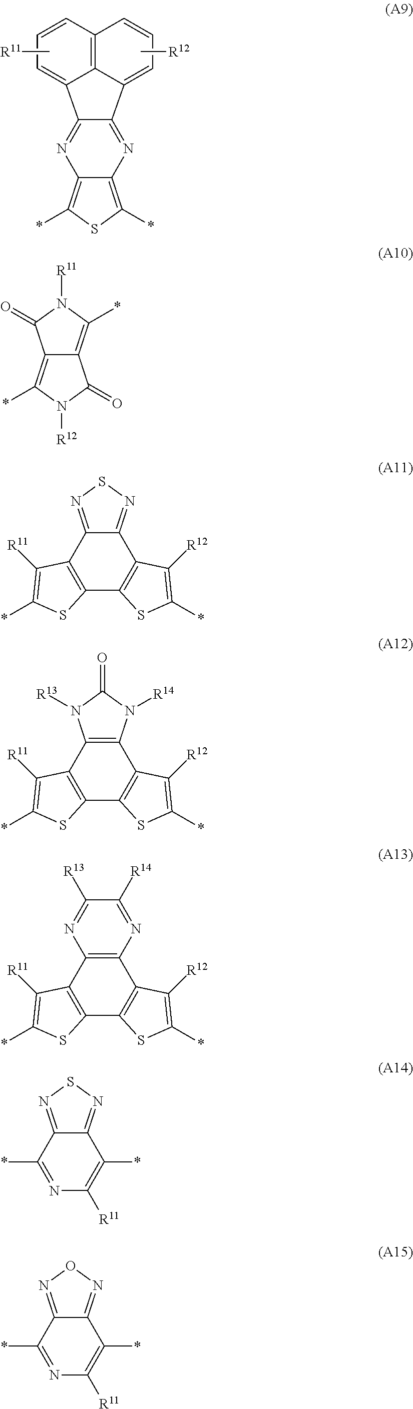

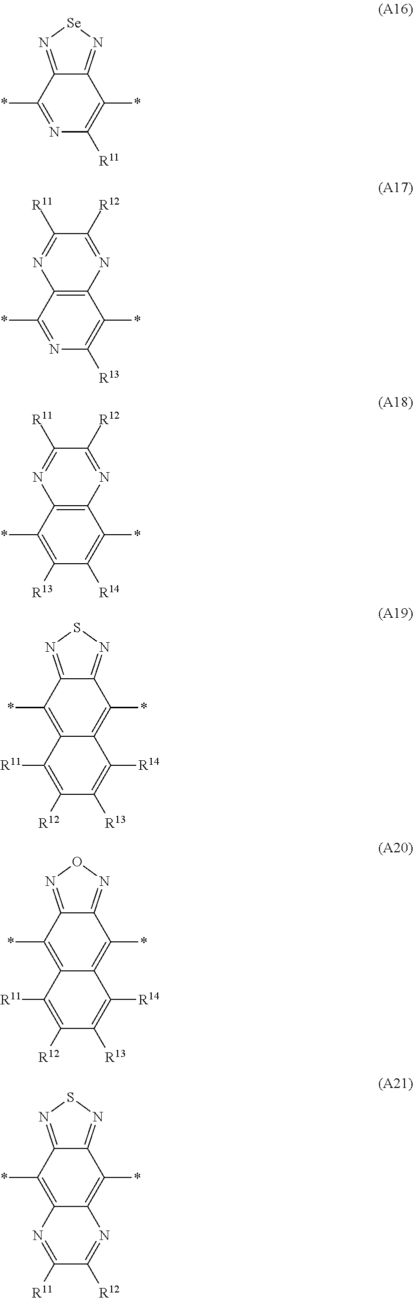

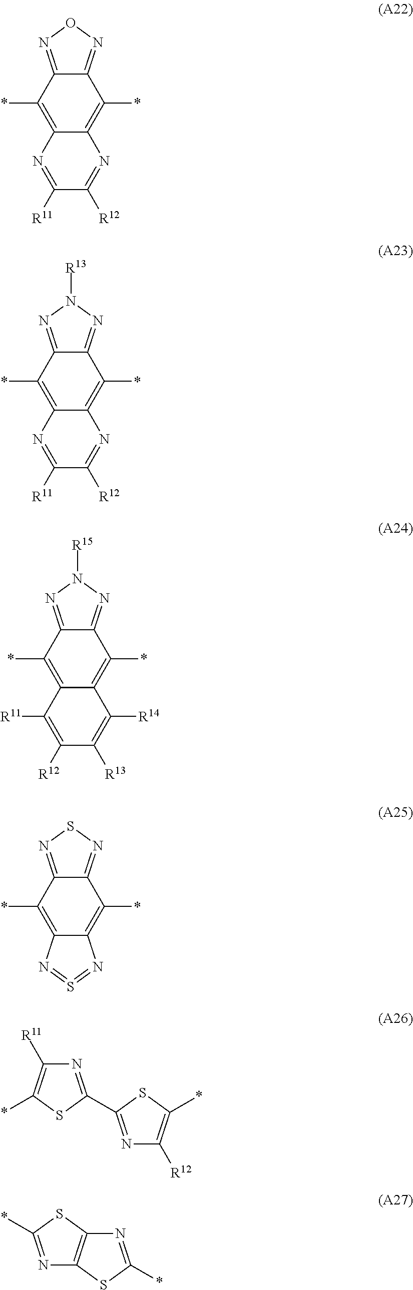

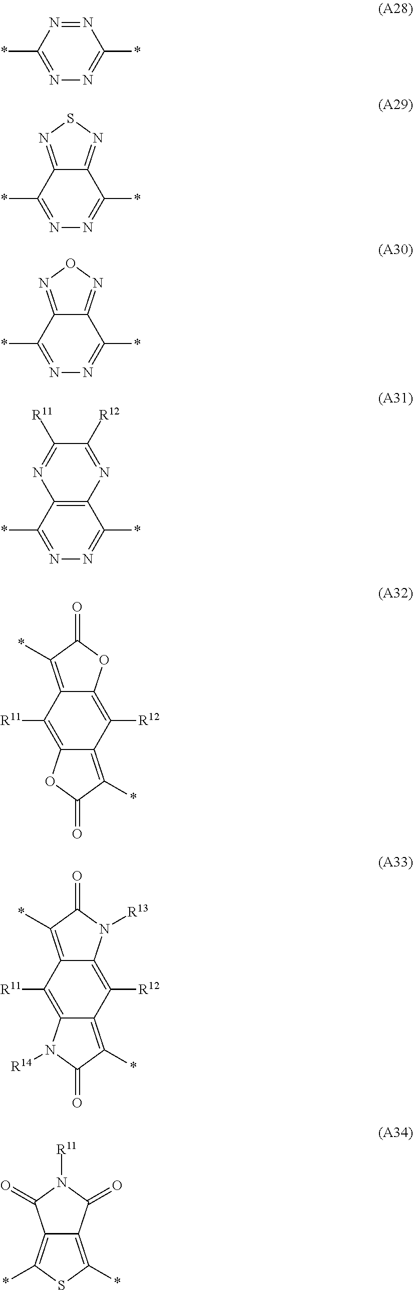

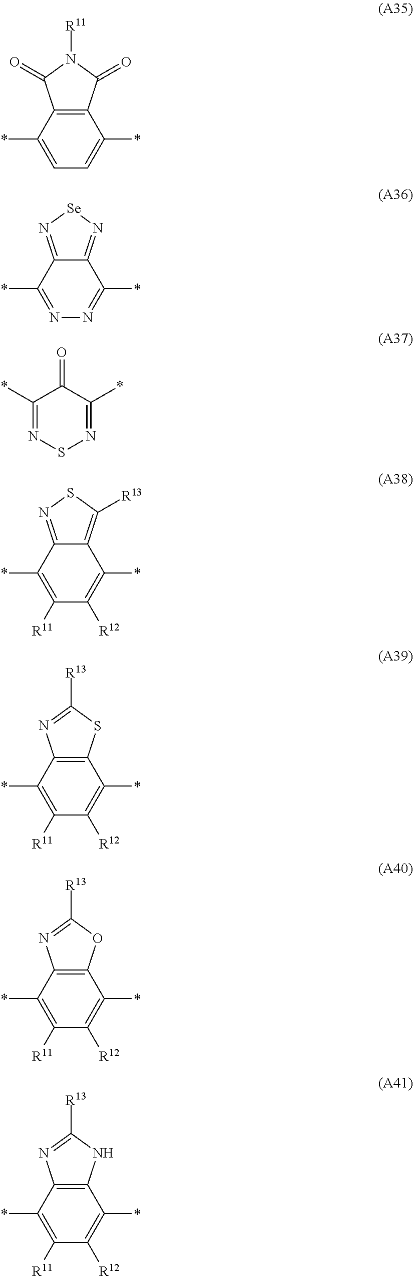

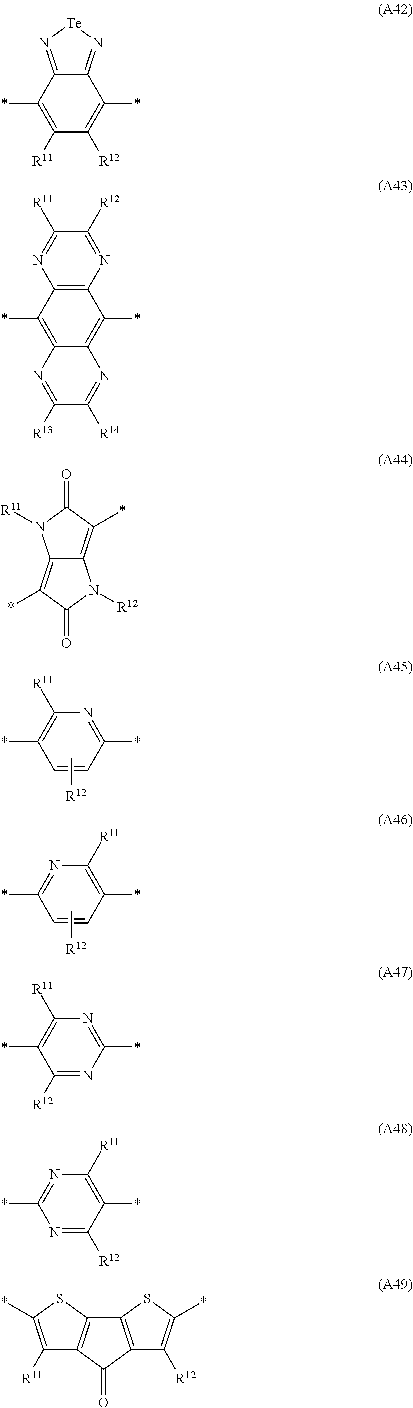

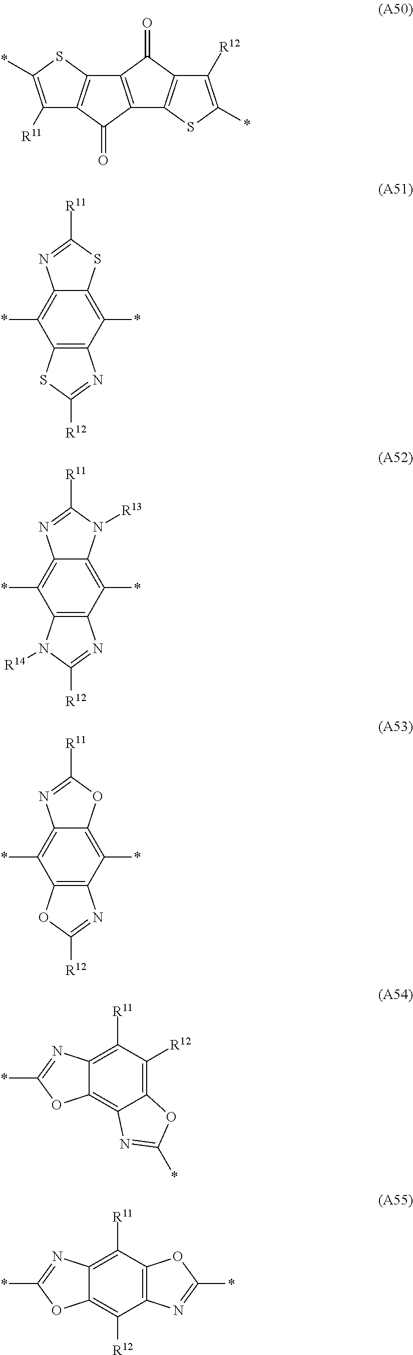

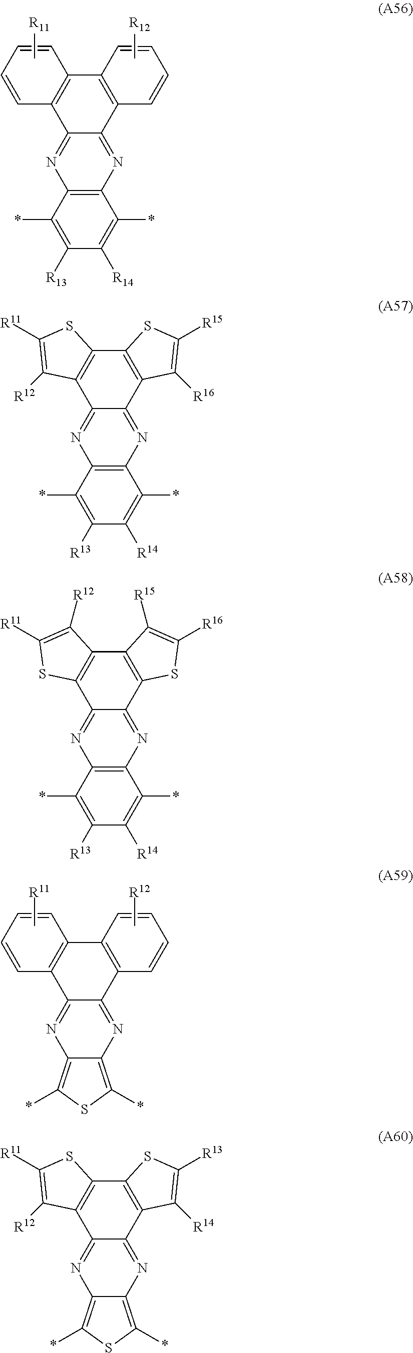

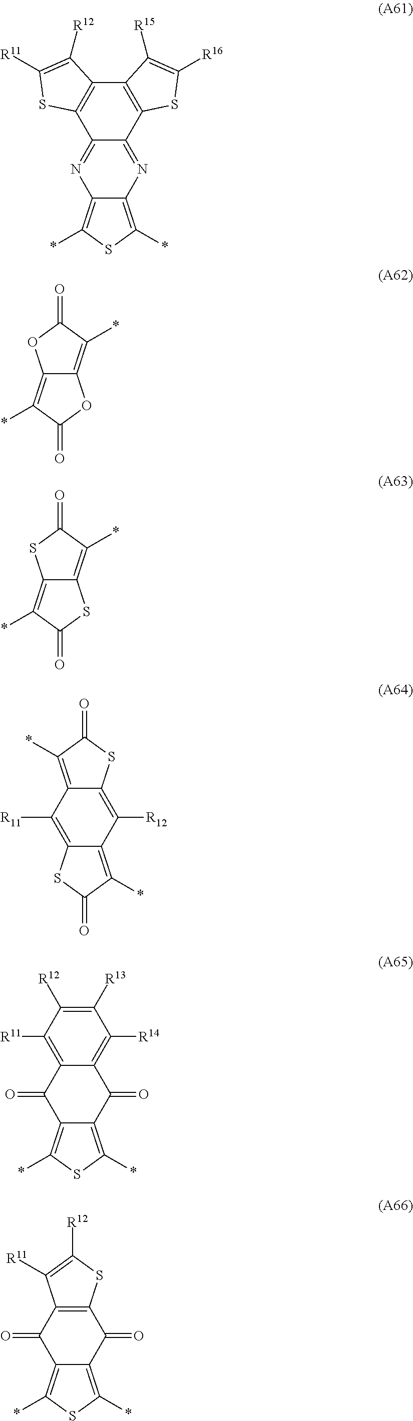

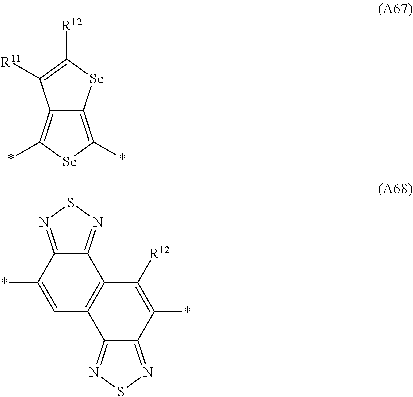

wherein one of X¹¹ and X¹² is S and the other is Se, and R¹¹, R¹², R¹³, R¹⁴ and R¹⁵ independently of each other denote H or have one of the meanings of R¹ as defined above and below.

Small molecule compounds and oligomers according to the present invention are preferably selected of formula VII)

R^{t1}-(Ar⁴)ₑ—(Ar⁵)_{f}—[(Ar⁶)_{g}—(Ar⁷)ₕ—U—(Ar⁸)ᵢ—(Ar⁹)ₖ]ₚ—(Ar¹⁰)ₗ—(Ar¹¹)ₒ—R^{t2} VII

wherein
U is a unit of formula I as defined above,
Ar⁴⁻¹² independently of each other denote —CY¹═CY²—, —C≡C—, or aryl or heteroaryl that has 5 to 30 ring atoms and is unsubstituted or substituted by one or more groups R¹ as defined in formula I, and one or more of Ar⁴⁻¹² may also denote U,
Y¹, Y² independently of each other denote H, F, Cl or CN,
R^{t1, t2} independently of each other denote H, F, Cl, Br, —CN, —CF₃, R, —CF₂—R, —O—R, —S—R, —SO₂—R, —SO₃—R —C(O)—R, —C(S)—R, —C(O)—CF₂—R, —C(O)—OR, —C(S)—OR, —O—C(O)—R, —O—C(S)—R, —C(O)—SR, —S—C(O)—R, —C(O)NRR′, —NR′—C(O)—R, —NHR, —NRR′, —CR′═CR″R′″, —C≡C—R′, —C≡C—SiR′R″R′″, —SiR′R″R′″, —CH═C(CN)—C(O)—OR, —CH═C(COOR)₂, CH═C(CONRR′)₂, CH═C(CN)(Ar¹²),

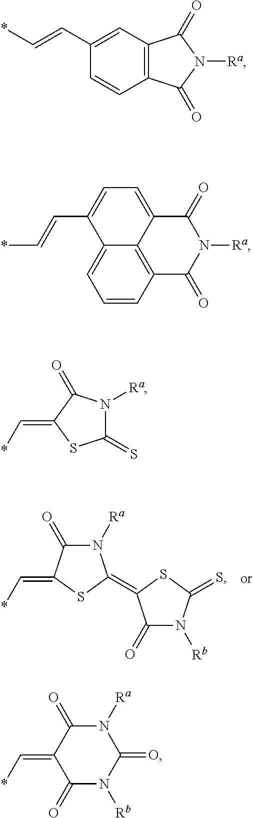

R^{a}, R^{b} are independently of each other aryl or heteroaryl, each having from 4 to 30 ring atoms and being unsubstituted or substituted with one or more groups R or R¹,
Ar¹² is aryl or heteroaryl, each having from 4 to 30 ring atoms and being unsubstituted or substituted with one or more groups R¹,
R is alkyl with 1 to 30 C atoms which is straight-chain, branched or cyclic, and is unsubstituted, substituted with one or more F or Cl atoms or CN groups, or perfluorinated, and in which one or more C atoms are optionally replaced by —O—, —S—, —C(O)—, —C(S)—, —SiR⁰R⁰⁰—, —NR⁰R⁰⁰—, —CHR⁰═CR⁰⁰— or —C≡C— such that O- and/or S-atoms are not directly linked to each other,
R⁰, R⁰⁰ independently of each other denote H or C₁₋₁₀ alkyl,
R′, R″, R′″ independently of each other have one of the meanings of R or denote H,
e, f, g, h, i, k, l, o are independently of each other 0 or 1, with at least one of e, f, g, h, i, k, l, o being 1,
p is 1, 2or 3.

Especially preferred groups Ar¹⁻¹² in the polymers and small molecules according to the present invention are selected from the following formulae:

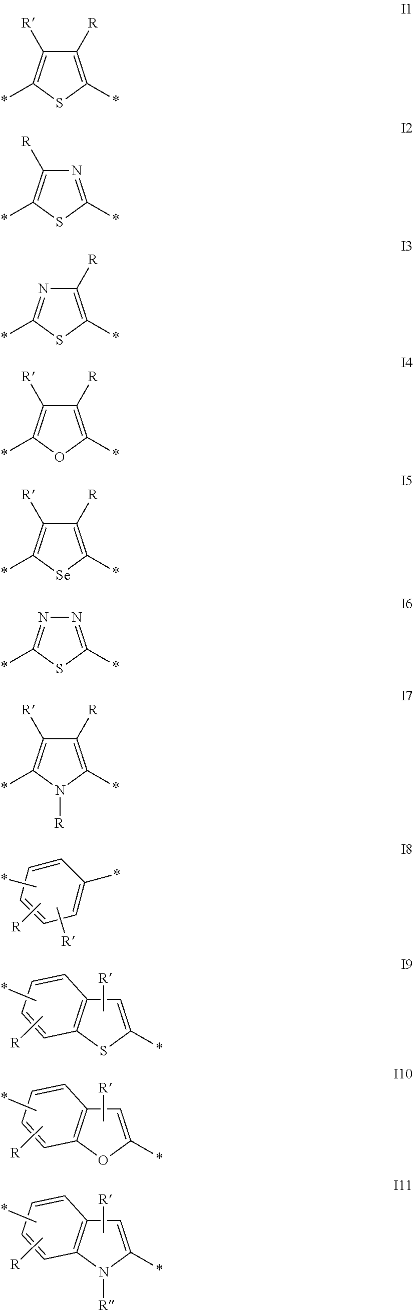

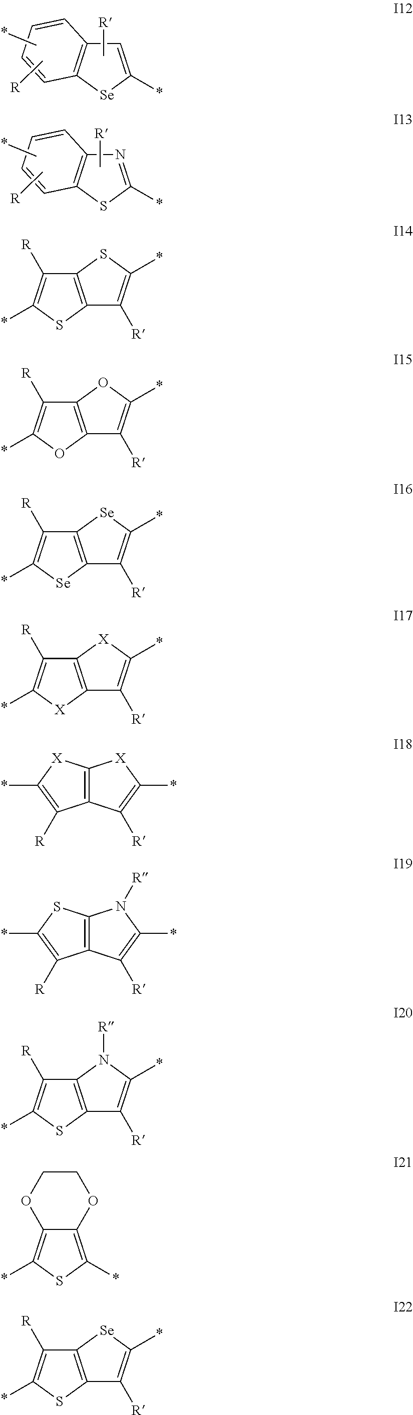

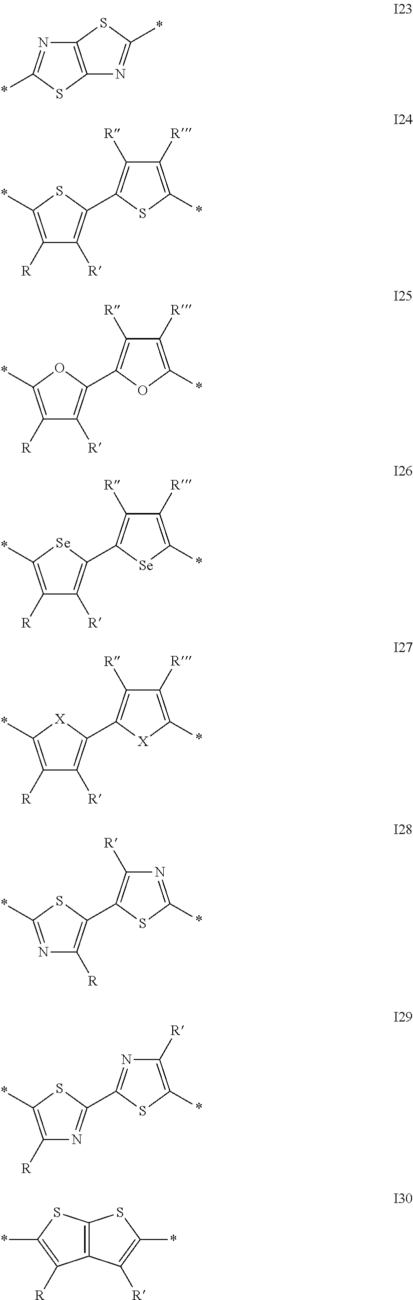

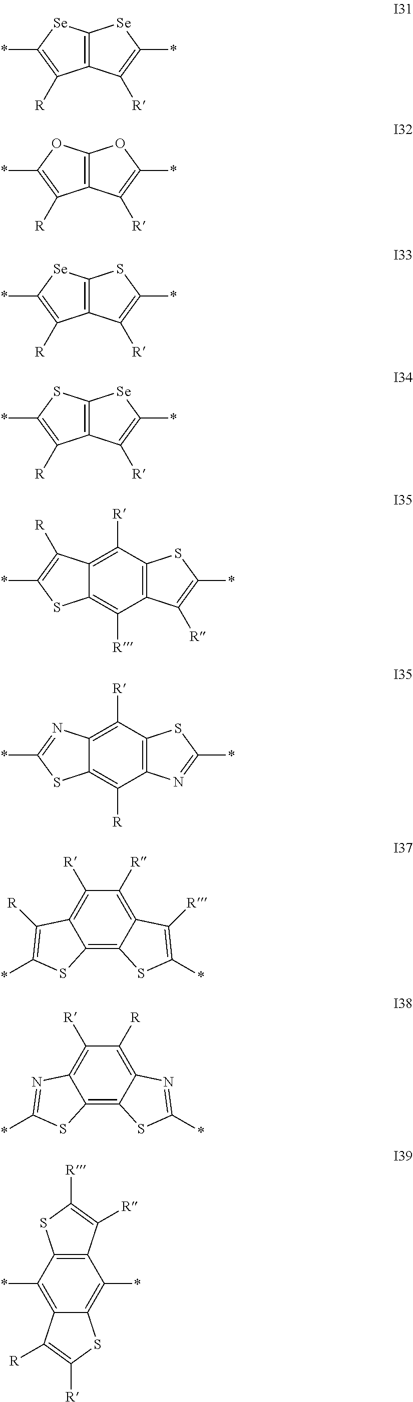

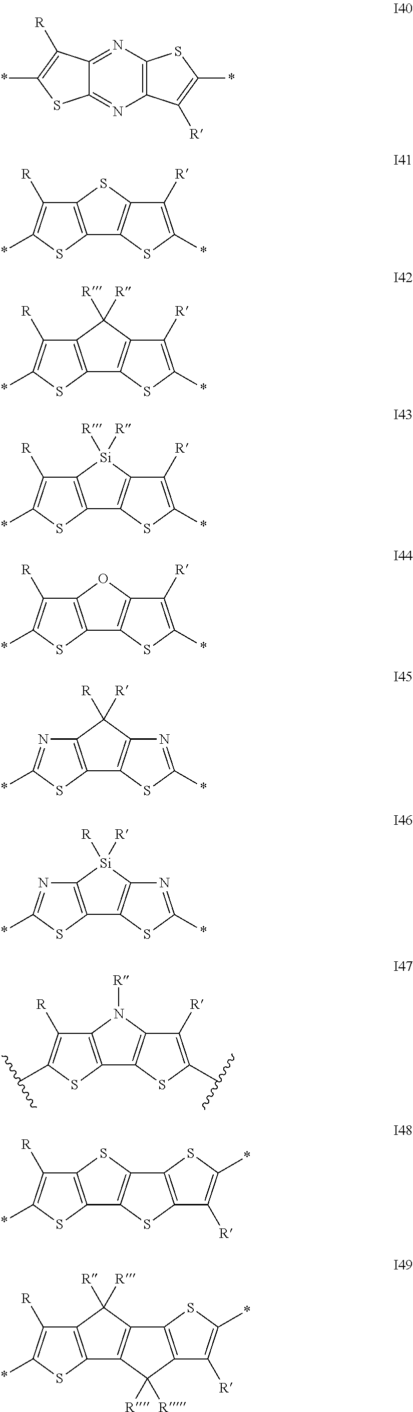

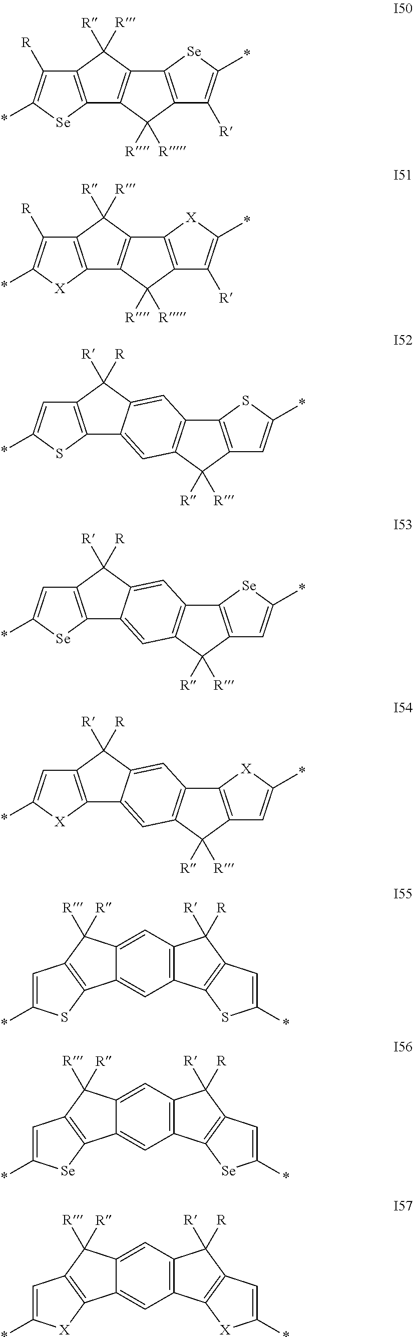

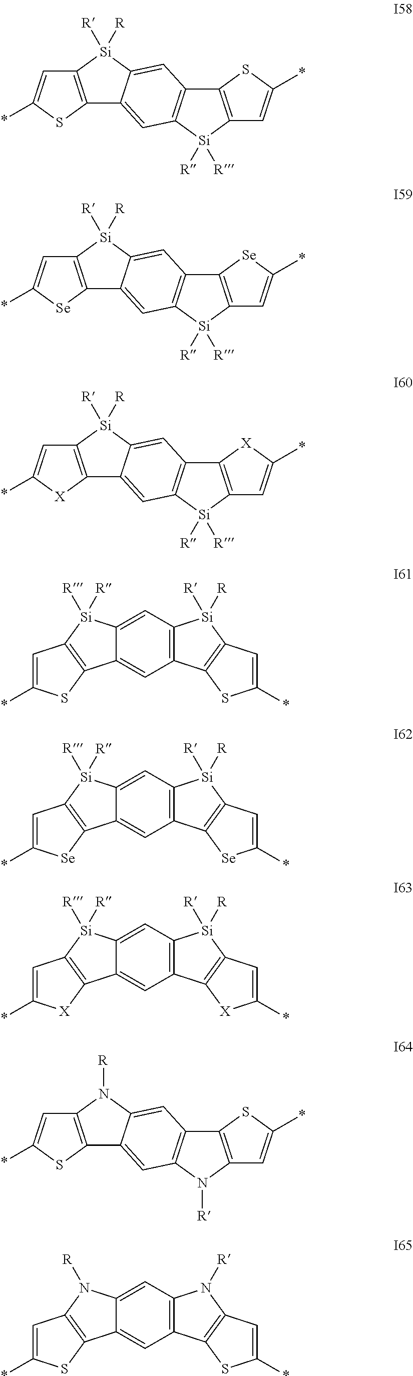

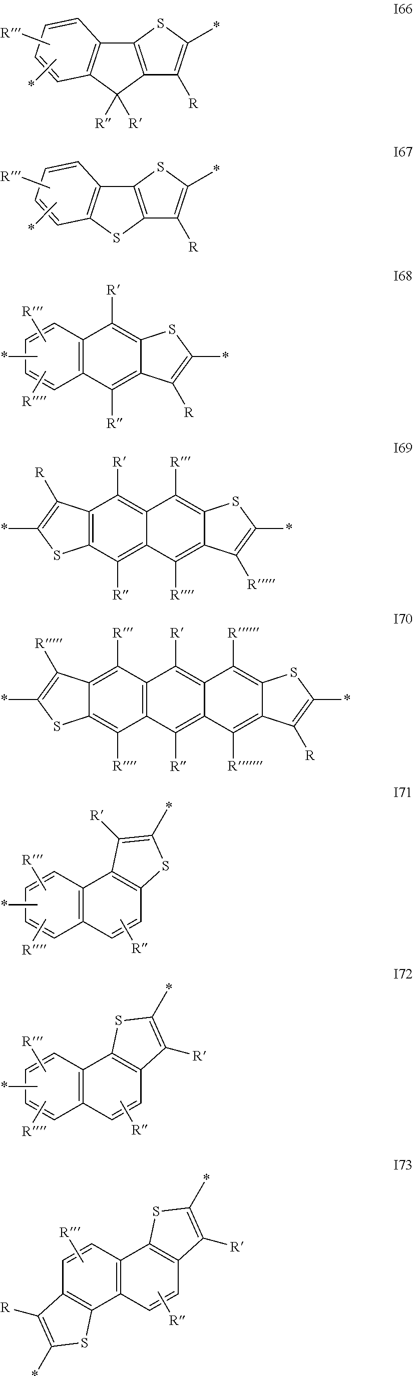

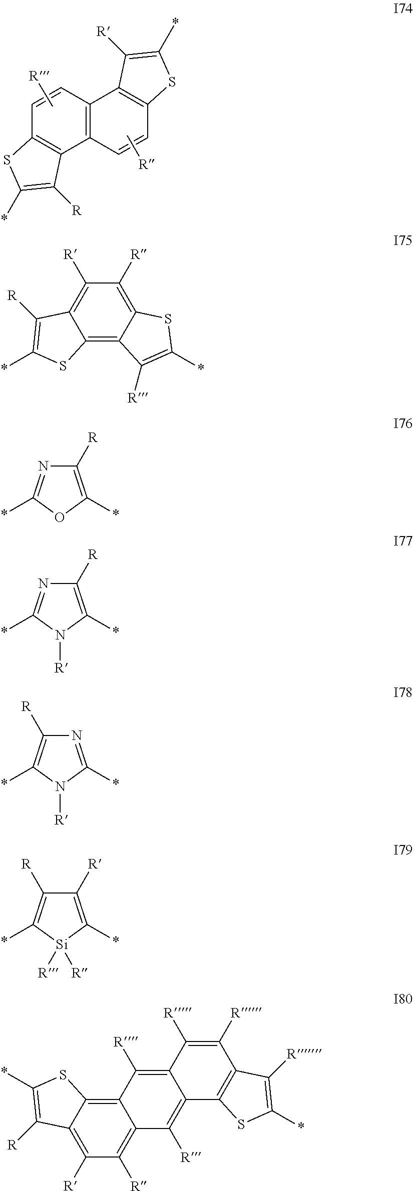

wherein R, R′, R″, R′″, R″″, R″″′, R″″″, R″″″′ and R″″″′ have one of the meanings of R¹ as given above.

Further preferred are repeating units, monomers and polymers of formulae I-VI and their subformulae selected from the following list of embodiments:
y is ≧0 and ≦1,
z is ≧0 and ≦1,
X¹ and X² are S,
X¹ and X² are O,
n is at least 5, preferably at least 10, very preferably at least 50, and up to 2,000, preferably up to 500.
M_{w} is at least 5,000, preferably at least 8,000, very preferably at least 10,000, and preferably up to 300,000, very preferably up to 100,000,
one of R¹ and R² is H and the other is different from H,
R¹ and R² are different from H,
R¹ and/or R² are independently of each other selected from the group consisting of primary alkyl with 1 to 30 C atoms, secondary alkyl with 3 to 30 C atoms, and tertiary alkyl with 4 to 30 C atoms, wherein in all these groups one or more H atoms are optionally replaced by F,
R¹ and/or R² are independently of each other selected from the group consisting of aryl and heteroaryl, each of which is optionally fluorinated, alkylated or alkoxylated and has 4 to 30 ring atoms,
R¹ and/or R² are independently of each other selected from the group consisting of primary alkoxy or sulfanylalkyl with 1 to 30 C atoms, secondary alkoxy or sulfanylalkyl with 3 to 30 C atoms, and tertiary alkoxy or sulfanylalkyl with 4 to 30 C atoms, wherein in all these groups one or more H atoms are optionally replaced by F,
R¹ and/or R² are independently of each other selected from the group consisting of aryloxy and heteroaryloxy, each of which is optionally alkylated or alkoxylated and has 4 to 30 ring atoms,
R¹ and/or R² are independently of each other selected from the group consisting of alkylcarbonyl, alkoxycarbonyl and alkylcarbonyloxy, all of which are straight-chain or branched, are optionally fluorinated, and have from 1 to 30 C atoms,
R¹ and/or R² denote independently of each other F, Cl, Br, I, CN, R⁹, —C(O)—R⁹, —C(O)—O—R⁹, or —O—C(O)—R⁹, —SO₂—R⁹, —SO₃—R⁹, wherein R⁹ is straight-chain, branched or cyclic alkyl with 1 to 30 C atoms, in which one or more non-adjacent C atoms are optionally replaced by —O—, —S—, —C(O)—, —C(O)—O—, —O—C(O)—, —O—C(O)—O—, —SO₂—, —SO₃—, —CR⁰═CR⁰⁰— or —C≡C— and in which one or more H atoms are optionally replaced by F, Cl, Br, I or CN, or R⁹ is aryl or heteroaryl having 4 to 30 ring atoms which is unsubstituted or which is substituted by one or more halogen atoms or by one or more groups R¹ as defined above,
R⁰ and R⁰⁰ are selected from H or C₁-C₁₀-alkyl,
R⁵ and R⁶ are selected from H, halogen, —CH₂Cl, —CHO, —CH═CH₂—SiR′R″R′″, —SnR′R″R′″, —BR′R″, —B(OR′)(OR″), —B(OH)₂, P-Sp, C₁-C₂₀-alkyl, C₁-C₂₀-alkoxy, C₂-C₂₀-alkenyl, C₁-C₂₀-fluoroalkyl and optionally substituted aryl or heteroaryl, preferably phenyl,
R⁷ and R⁸ are, preferably independently of each other, selected from the group consisting of Cl, Br, I, O-tosylate, O-triflate, O-mesylate, O-nonaflate, —SiMe₂F, —SiMeF₂, —O—SO₂Z¹, —B(OZ²)₂, —CZ³═C(Z⁴)₂, —C≡CH, C≡CSi(Z¹)₃, —ZnX⁰ and —Sn(Z⁴)₃, wherein X⁰ is halogen, Z¹⁻⁴ are selected from the group consisting of alkyl and aryl, each being optionally substituted, and two groups Z² may also form a cyclic group.

The compounds of the present invention can be synthesized according to or in analogy to methods that are known to the skilled person and are described in the literature. Other methods of preparation can be taken from the examples. For example, the polymers can be suitably prepared by aryl-aryl coupling reactions, such as Yamamoto coupling, Suzuki coupling, Stille coupling, Sonogashira coupling, Heck coupling or Buchwald coupling. Suzuki coupling, Stille coupling and Yamamoto coupling are especially preferred. The monomers which are polymerised to form the repeat units of the polymers can be prepared according to methods which are known to the person skilled in the art.

Preferably the polymers are prepared from monomers of formula VIa or VIb or their preferred subformulae as described above and below.

Another aspect of the invention is a process for preparing a polymer by coupling one or more identical or different monomeric units of formula I or monomers of formula VIa or VIb with each other and/or with one or more comonomers in a polymerisation reaction, preferably in an aryl-aryl coupling reaction.

Suitable and preferred comonomers are selected from the following formulae

R⁷—(Ar¹)ₐ-D-(Ar²)_{c}—R⁸ VIII

R⁷—Ar¹—R⁸ IX

R⁷—Ar³—R⁸ X

wherein Ar¹, Ar², Ar³, a and c have one of the meanings of formula IIa or one of the preferred meanings given above and below, A^{c} has one of the meanings of formula IIIa or one of the preferred meanings given above and below, and R⁷ and R⁸ have one of meanings of formula VI or one of the preferred meanings given above and below.

Very preferred is a process for preparing a polymer by coupling one or more monomers selected from formula VIa, VIb or formulae VI1-VI5 with one or more monomers of formula VIII, and optionally with one or more monomers selected from formula IX and X, in an aryl-aryl coupling reaction, wherein preferably R⁷ and R⁸ are selected from Cl, Br, I, —B(OZ²)₂ and —Sn(Z⁴)₃.

For example, preferred embodiments of the present invention relate to

a) a process of preparing a polymer by coupling a monomer of formula VI1

R⁷—Ar¹—U—Ar²—R⁸ VI1

with a monomer of formula IX

R⁷—Ar¹—R⁸ IX

in an aryl-aryl coupling reaction, or

b) a process of preparing a polymer by coupling a monomer of formula VI2

R⁷—U—R⁸ VI2

with a monomer of formula VIII1

R⁷—Ar¹-D-Ar²—R⁸ VIII1

in an aryl-aryl coupling reaction,

or

c) a process of preparing a polymer by coupling a monomer of formula VI2

R⁷—U—R⁸ VI2

with a monomer of formula VIII-2

R⁷-D-R⁸ VIII2

in an aryl-aryl coupling reaction, or

d) a process of preparing a polymer by coupling a monomer of formula VI2

R⁷—U—R⁸ VI2

with a monomer of formula VIII2

R⁷-D-R⁸ VIII2

and a monomer of formula IX

R⁷—Ar¹—R⁸ IX

in an aryl-aryl coupling reaction,

e) a process of preparing a polymer by coupling a monomer of formula VI1

R⁷—U—Ar¹—U—R⁸ VI5

with a monomer of formula IX

R⁷—Ar¹—R⁸ IX

in an aryl-aryl coupling reaction,

or

f) a process of preparing a polymer by coupling a monomer of formula VI2

R⁷—U—R⁸ VI2

with a monomer of formula IX

R⁷—Ar¹—R⁸ IX

and a monomer of formula X

R⁷—Ar³—R⁸ X

in an aryl-aryl coupling reaction,

wherein R⁷, R⁸, U, D, Ar^{1,2,3} are as defined in formula IIa, IIIa and VIa, and R⁷ and R⁸ are preferably selected from Cl, Br, I, —B(OZ²)₂ and —Sn(Z⁴)₃ as defined in formula VIa.

Preferred aryl-aryl coupling and polymerisation methods used in the processes described above and below are Yamamoto coupling, Kumada coupling, Negishi coupling, Suzuki coupling, Stille coupling, Sonogashira coupling, Heck coupling, C—H activation coupling, Ullmann coupling or Buchwald coupling. Especially preferred are Suzuki coupling, Negishi coupling, Stille coupling and Yamamoto coupling. Suzuki coupling is described for example in WO 00/53656 A1. Negishi coupling is described for example in J. Chem. Soc., Chem. Commun., 1977, 683-684. Yamamoto coupling is described in for example in T. Yamamoto et al., Prog. Polym. Sci., 1993, 17, 1153-1205, or WO 2004/022626 A1. For example, when using Yamamoto coupling, compounds of formula II having two reactive halide groups are preferably used. When using Suzuki coupling, compounds of formula II having two reactive boronic acid or boronic acid ester groups or two reactive halide groups are preferably used. When using Stille coupling, compounds of formula II having two reactive stannane groups or two reactive halide groups are preferably used. When using Negishi coupling, compounds of formula II having two reactive organozinc groups or two reactive halide groups are preferably used.

Preferred catalysts, especially for Suzuki, Negishi or Stille coupling, are selected from Pd(0) complexes or Pd(II) salts. Preferred Pd(0) complexes are those bearing at least one phosphine ligand such as Pd(Ph₃P)₄. Another preferred phosphine ligand is tris(ortho-tolyl)phosphine, i.e. Pd(o-Tol₃P)₄. Preferred Pd(II) salts include palladium acetate, i.e. Pd(OAc)₂. Alternatively the Pd(0) complex can be prepared by mixing a Pd(0) dibenzylideneacetone complex, for example tris(dibenzyl-ideneacetone)dipalladium(0), bis(dibenzylideneacetone)palladium(0), or Pd(II) salts e.g. palladium acetate, with a phosphine ligand, for example triphenylphosphine, tris(ortho-tolyl)phosphine or tri(tert-butyl)phosphine. Suzuki coupling is performed in the presence of a base, for example sodium carbonate, potassium carbonate, lithium hydroxide, potassium phosphate or an organic base such as tetraethylammonium carbonate or tetraethylammonium hydroxide. Yamamoto coupling employs a Ni(0) complex, for example bis(1,5-cyclooctadienyl) nickel(0).

As alternatives to halogens as described above, leaving groups of formula —O—SO₂Z¹ can be used wherein Z¹ is as described above. Particular examples of such leaving groups are tosylate, mesylate and triflate.

Especially suitable and preferred synthesis methods of the repeating units, small molecules, monomers and polymers of formulae I-VII and their subformulae are illustrated in the synthesis schemes shown hereinafter, wherein R has one of the meanings of R¹ given above.

The generic preparation of symmetric 1,5-dimethyl-1,5-dihydro-[1,5]naphthyridine-2,6-dione core has been described for example in H. Rapoport, A. D. Batcho, J. Org. Chem. 1963, 28, 1753-1759, and illustrated in Scheme 1.

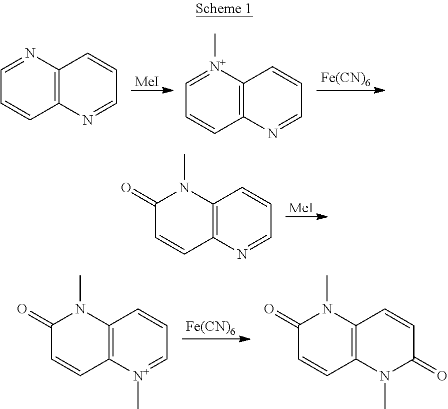

Synthesis of the 1,5-disubstituted-1,5-dihydro-[1,5]naphthyridine-2,6-dione core could be achieved, for example, by the following methods described in Schemes 2 and 3 (Frydman, B. Los, M. Rapoport, H. J. Org. Chem., 1971, 36, 450-454), 4 (Singh, A. N. Thummel, R. P., Inorg. Chem., 2009, 48, 6459-6470) and 5 (Bowers, S. Truong, A. P. Neitz, R. J. Flom, R. K. Sealy, J. M. Probst, G. D. Quincy, D. Peterson, B. Chan, W. Galemmo Jr., R. A. Konradi, A. W. Sham, H. L. Tóth, G. Pan, H. Lin, M. Yao, N. Artis, D. R. Zhang, H. Chen, L. Dryer, M. Samant, B. Zmolek, W. Wong, K. Lorentzen, C. Goldbach, E. Tonn, G. Quinn, K. P. Sauer, J-M. Wright, S. Powell, K. Ruslim, L. Ren, Z. Bard, F. Yednock, T. A. Griswold-Prenner, I. Bioorg. Med. Chem. Lett. 2011, 21, 5521-5527) to prepare the required precursors to polymers and compounds.

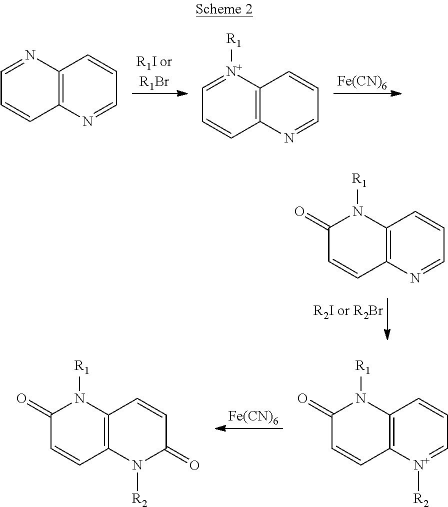

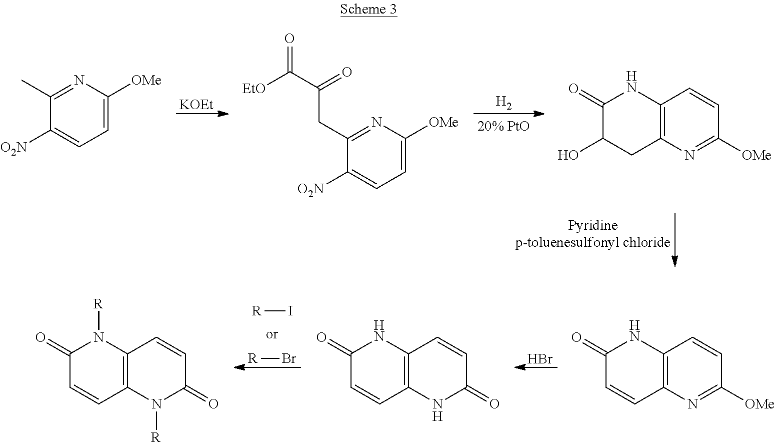

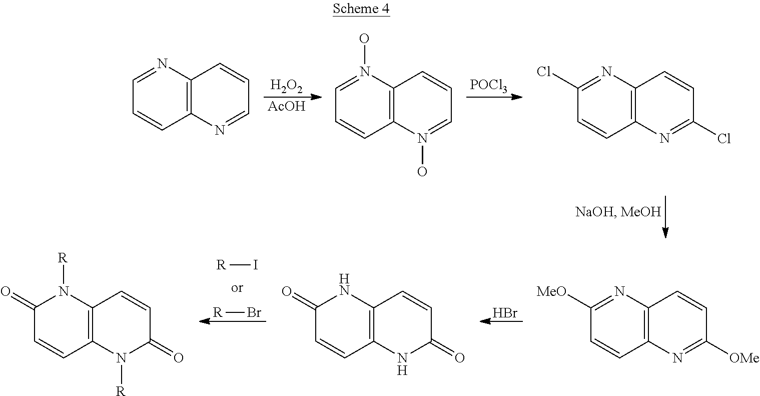

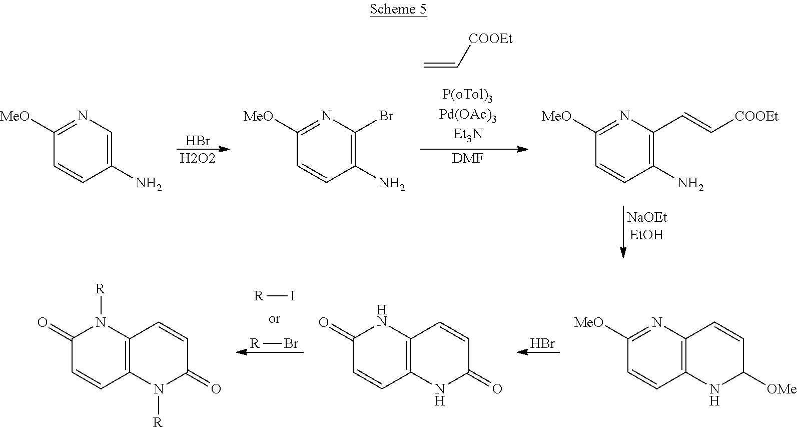

Following the generic dihydro-[1,5]naphthyridine-2,6-dione core synthesis, further substitution can be done as described in Scheme 6.

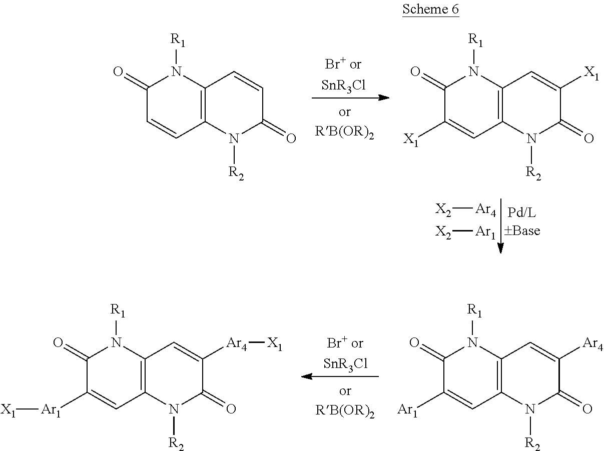

The dithione can be accessed as shown in Scheme 7.

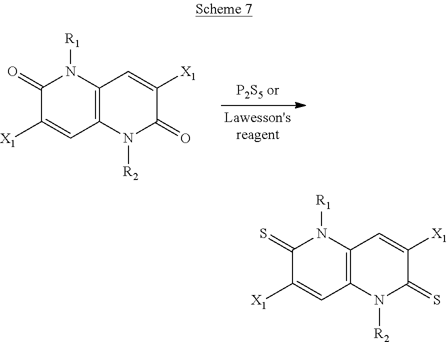

Synthesis schemes for alternating co-polymers of the 1,5-disubstituted-1,5-dihydro-[1,5]naphthyridine-2,6-dione are shown in Scheme 8.

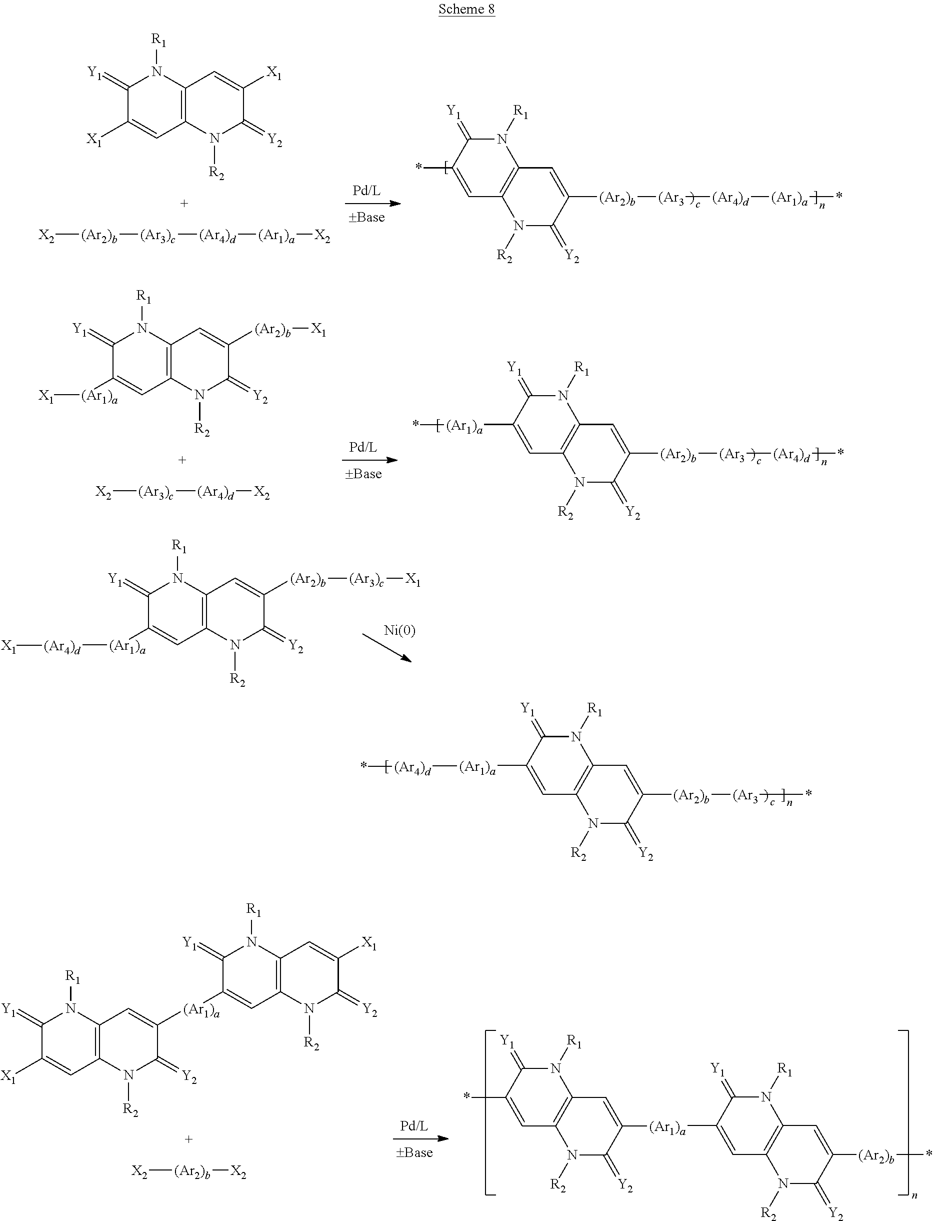

where Y1 and Y2 is in each occurrence identically or differently O or S, X1=Br and X2=SnR₃ or X1=Br and X2=B(OR)₂ or X1=SnR₃ and X2=Br or X1=B(OR)₂ and X2=Br or X1=Br or Cl, Arₓ is an optionally substituted aryl or heteroaryl and a+b+c+d≧0

Synthesis schemes for the statistical block co-polymers of the 1,5-disubstituted-1,5-dihydro-[1,5]naphthyridine-2,6-dione are shown in Scheme 9.

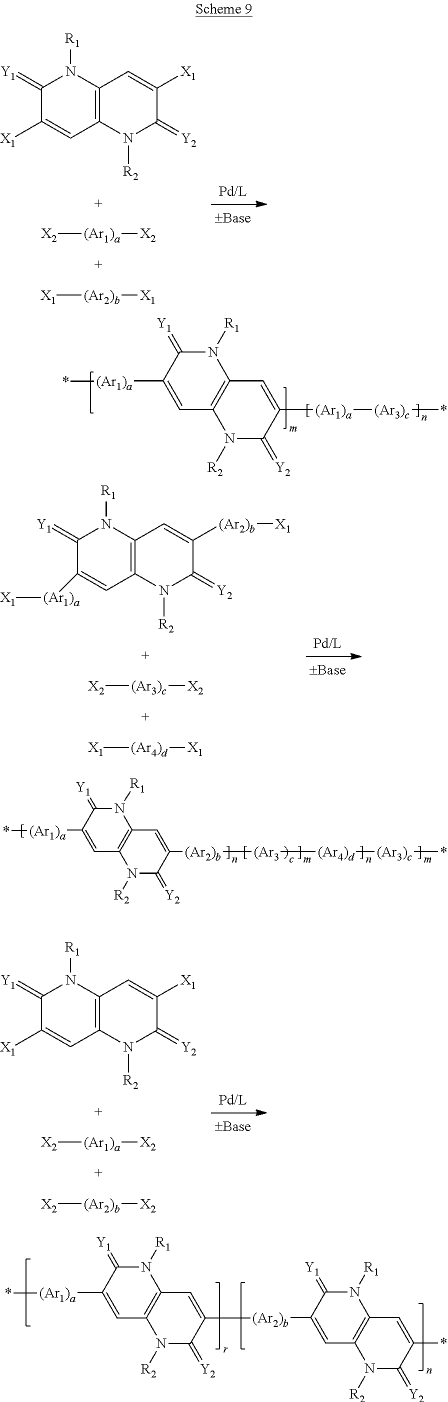

where Y1 and Y2 is in each occurrence identically or differently O or S, X1=Br and X2=SnR₃, X1=Br and X2=B(OR)₂ or X1=SnR₃ and X2=Br or X1=B(OR)₂ and X2=Br, Arₓ is in each occurrence identically or differently, and independently of each other, an optionally substituted aryl or heteroaryl and a, b, c and d are equal or greater than 1.

Synthesis schemes for the 1,5-disubstituted-1,5-dihydro-[1,5]naphthyridine-2,6-dione core based compounds are shown in Scheme 10.

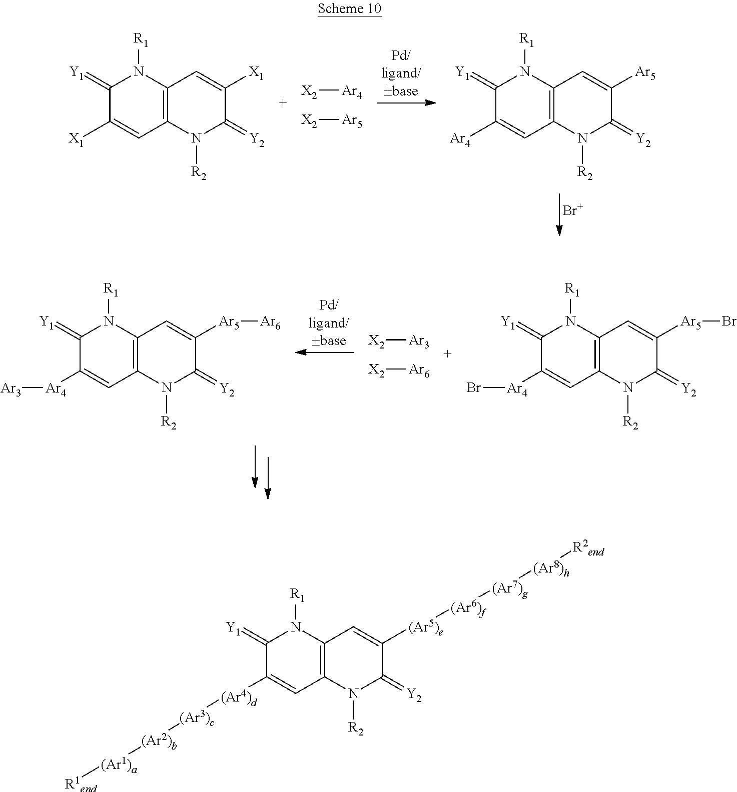

where Y1 and Y2 is in each occurrence identically or differently O or S, X1=Br and X2=SnR₃ or B(OR)₂ or X1=SnR₃ and X2=Br or X1=B(OR)₂ and X2=Br and where Ar₅—Ar₆—Ar₇—Ar₈—R²_{end} is identical to Ar₄—Ar₃—Ar₂—Ar₁—R¹_{end.}

Alternatively the 1,5-disubstituted-1,5-dihydro-[1,5]naphthyridine-2,6-dione based organic semiconductor can be obtained via a convergent synthesis strategy as shown in Scheme 11.

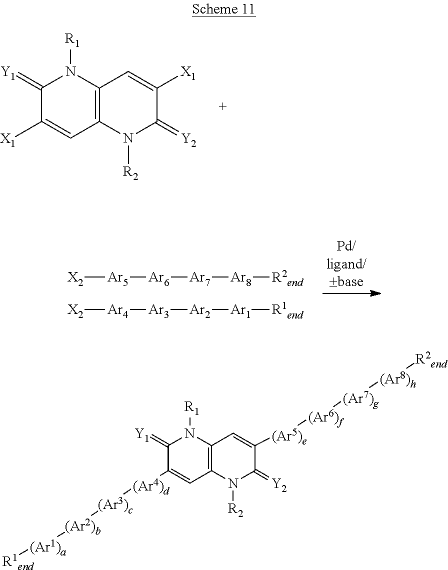

where Y1 and Y2 is in each occurrence identically or differently O or S, X1=Br and X2=SnR₃ or B(OR)₂ or X1=SnR₃ and X2=Br or X1=B(OR)₂ and X2=Br.

Alternatively the asymmetric 1,5-disubstituted-1,5-dihydro-[1,5]naphthyridine-2,6-dione based organic semiconductor can be obtained via a convergent synthesis strategy as shown in Scheme 12.

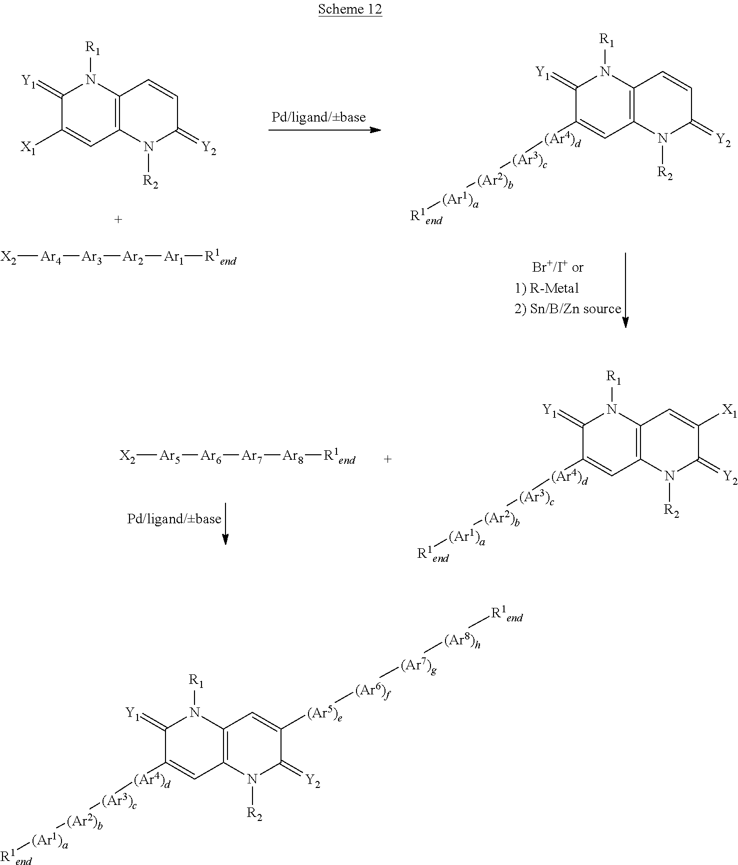

where Y1 and Y2 is in each occurrence identically or differently O or S, X1=Br and X2=SnR₃ or B(OR)₂ or X1=SnR₃ and X2=Br or X1=B(OR)₂ and X2=Br.

The synthesis scheme for asymmetric organic semiconductor compounds containing multiple 1,5-disubstituted-1,5-dihydro-[1,5]naphthyridine-2,6-dione units is shown in Scheme 13.

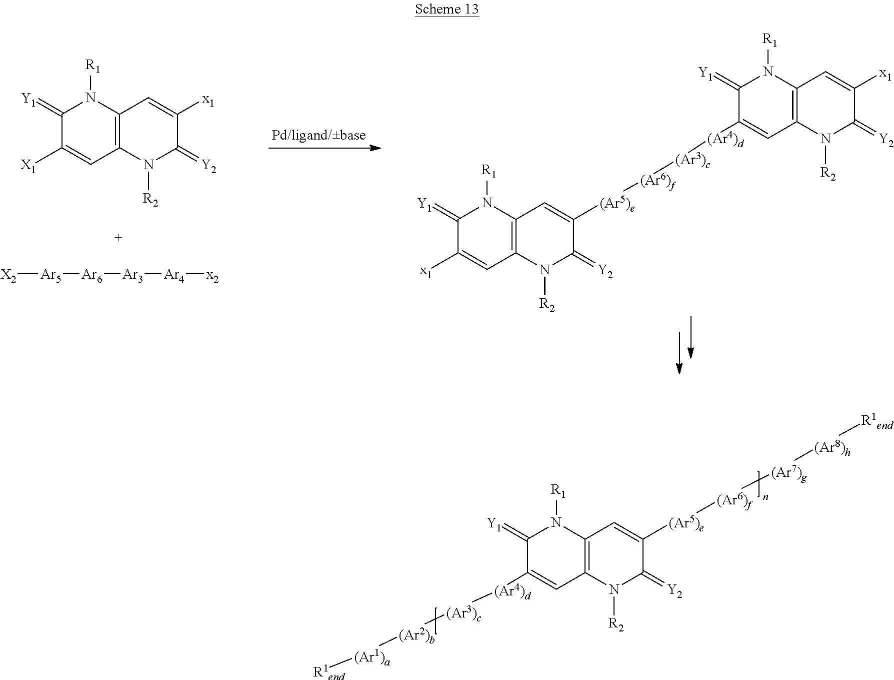

where Y1 and Y2 is in each occurrence identically or differently O or S, X1=Br and X2=SnR₃ or B(OR)₂ or X1=SnR₃ and X2=Br or X1=B(OR)₂ and X2=Br, and 1<n≦10.

When preparing small molecules, further substitution can be added to the 1,5-disubstituted-1,5-dihydro-[1,5]naphthyridine-2,6-dione core at the R^{x}_{end} substitution after the 1,5-disubstituted-1,5-dihydro-[1,5]naphthyridine-2,6-dione core organic semiconductors have been prepared as shown in Scheme 14.

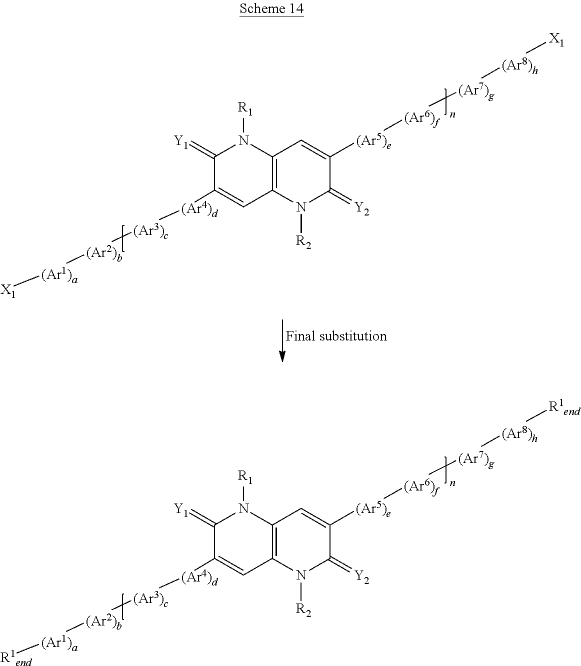

The novel methods of preparing small molecules, monomers and polymers as described above and below are another aspect of the invention.

The compounds and polymers according to the present invention can also be used in mixtures or polymer blends, for example together with monomeric compounds or together with other polymers having charge-transport, semiconducting, electrically conducting, photoconducting and/or light emitting semiconducting properties, or for example with polymers having hole blocking or electron blocking properties for use as interlayers or charge blocking layers in OLED devices. Thus, another aspect of the invention relates to a polymer blend comprising one or more polymers according to the present invention and one or more further polymers having one or more of the above-mentioned properties. These blends can be prepared by conventional methods that are described in prior art and known to the skilled person. Typically the polymers are mixed with each other or dissolved in suitable solvents and the solutions combined.

Another aspect of the invention relates to a formulation comprising one or more small molecules, polymers, mixtures or polymer blends as described above and below and one or more organic solvents.

Preferred solvents are aliphatic hydrocarbons, chlorinated hydrocarbons, aromatic hydrocarbons, ketones, ethers and mixtures thereof. Additional solvents which can be used include 1,2,4-trimethylbenzene, 1,2,3,4-tetra-methyl benzene, pentylbenzene, mesitylene, cumene, cymene, cyclohexylbenzene, diethylbenzene, tetralin, decalin, 2,6-lutidine, 2-fluoro-m-xylene, 3-fluoro-o-xylene, 2-chlorobenzotrifluoride, N,N-dimethylformamide, 2-chloro-6-fluorotoluene, 2-fluoroanisole, anisole, 2,3-dimethylpyrazine, 4-fluoroanisole, 3-fluoroanisole, 3-trifluoro-methylanisole, 2-methylanisole, phenetol, 4-methylanisole, 3-methylanisole, 4-fluoro-3-methylanisole, 2-fluorobenzonitrile, 4-fluoroveratrol, 2,6-dimethylanisole, 3-fluorobenzo-nitrile, 2,5-dimethylanisole, 2,4-dimethylanisole, benzonitrile, 3,5-dimethyl-anisole, N,N-dimethylaniline, ethyl benzoate, 1-fluoro-3,5-dimethoxy-benzene, 1-methylnaphthalene, N-methylpyrrolidinone, 3-fluorobenzo-trifluoride, benzotrifluoride, dioxane, trifluoromethoxy-benzene, 4-fluorobenzotrifluoride, 3-fluoropyridine, toluene, 2-fluoro-toluene, 2-fluorobenzotrifluoride, 3-fluorotoluene, 4-isopropylbiphenyl, phenyl ether, pyridine, 4-fluorotoluene, 2,5-difluorotoluene, 1-chloro-2,4-difluorobenzene, 2-fluoropyridine, 3-chlorofluorobenzene, 1-chloro-2,5-difluorobenzene, 4-chlorofluorobenzene, chloro-benzene, o-dichlorobenzene, 2-chlorofluorobenzene, p-xylene, m-xylene, o-xylene or mixture of o-, m-, and p-isomers. Solvents with relatively low polarity are generally preferred. For inkjet printing solvents and solvent mixtures with high boiling temperatures are preferred. For spin coating alkylated benzenes like xylene and toluene are preferred.

Examples of especially preferred solvents include, without limitation, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, N,N-dimethylformamide, dimethylacetamide, dimethylsulfoxide, tetraline, decaline, indane, methyl benzoate, ethyl benzoate, mesitylene and/or mixtures thereof.

The concentration of the compounds or polymers in the solution is preferably 0.1 to 10% by weight, more preferably 0.5 to 5% by weight. Optionally, the solution also comprises one or more binders to adjust the rheological properties, as described for example in WO 2005/055248 A1.

After the appropriate mixing and ageing, solutions are evaluated as one of the following categories: complete solution, borderline solution or insoluble. The contour line is drawn to outline the solubility parameter-hydrogen bonding limits dividing solubility and insolubility. ‘Complete’ solvents falling within the solubility area can be chosen from literature values such as published in “Crowley, J. D., Teague, G. S. Jr and Lowe, J. W. Jr., Journal of Paint Technology, 1966, 38 (496), 296”. Solvent blends may also be used and can be identified as described in “Solvents, W.H.Ellis, Federation of Societies for Coatings Technology, p 9-10, 1986”. Such a procedure may lead to a blend of ‘non’ solvents that will dissolve both the polymers of the present invention, although it is desirable to have at least one true solvent in a blend.

The compounds and polymers according to the present invention can also be used in patterned OSC layers in the devices as described above and below. For applications in modern microelectronics it is generally desirable to generate small structures or patterns to reduce cost (more devices/unit area), and power consumption. Patterning of thin layers comprising a polymer according to the present invention can be carried out for example by photolithography, electron beam lithography or laser patterning.

For use as thin layers in electronic or electrooptical devices the compounds, polymers, polymer blends or formulations of the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot dye coating or pad printing.

Ink jet printing is particularly preferred when high resolution layers and devices needs to be prepared. Selected formulations of the present invention may be applied to prefabricated device substrates by ink jet printing or microdispensing. Preferably industrial piezoelectric print heads such as but not limited to those supplied by Aprion, Hitachi-Koki, InkJet Technology, On Target Technology, Picojet, Spectra, Trident, Xaar may be used to apply the organic semiconductor layer to a substrate. Additionally semi-industrial heads such as those manufactured by Brother, Epson, Konica, Seiko Instruments Toshiba TEC or single nozzle microdispensers such as those produced by Microdrop and Microfab may be used.

In order to be applied by ink jet printing or microdispensing, the compounds or polymers should be first dissolved in a suitable solvent. Solvents must fulfil the requirements stated above and must not have any detrimental effect on the chosen print head. Additionally, solvents should have boiling points >100° C., preferably >140° C. and more preferably >150° C. in order to prevent operability problems caused by the solution drying out inside the print head. Apart from the solvents mentioned above, suitable solvents include substituted and non-substituted xylene derivatives, di-C₁₋₂-alkyl formamide, substituted and non-substituted anisoles and other phenol-ether derivatives, substituted heterocycles such as substituted pyridines, pyrazines, pyrimidines, pyrrolidinones, substituted and non-substituted N,N-di-C₁₋₂-alkylanilines and other fluorinated or chlorinated aromatics.

A preferred solvent for depositing a compound or polymer according to the present invention by ink jet printing comprises a benzene derivative which has a benzene ring substituted by one or more substituents wherein the total number of carbon atoms among the one or more substituents is at least three. For example, the benzene derivative may be substituted with a propyl group or three methyl groups, in either case there being at least three carbon atoms in total. Such a solvent enables an ink jet fluid to be formed comprising the solvent with the compound or polymer, which reduces or prevents clogging of the jets and separation of the components during spraying. The solvent(s) may include those selected from the following list of examples: dodecylbenzene, 1-methyl-4-tert-butylbenzene, terpineol, limonene, isodurene, terpinolene, cymene, diethylbenzene. The solvent may be a solvent mixture, that is a combination of two or more solvents, each solvent preferably having a boiling point >100° C., more preferably >140° C. Such solvent(s) also enhance film formation in the layer deposited and reduce defects in the layer.

The ink jet fluid (that is mixture of solvent, binder and semiconducting compound) preferably has a viscosity at 20° C. of 1-100 mPa·s, more preferably 1-50 mPa·s and most preferably 1-30 mPa·s.

The polymer blends and formulations according to the present invention can additionally comprise one or more further components or additives selected for example from surface-active compounds, lubricating agents, wetting agents, dispersing agents, hydrophobing agents, adhesive agents, flow improvers, defoaming agents, deaerators, diluents which may be reactive or non-reactive, auxiliaries, colourants, dyes or pigments, sensitizers, stabilizers, nanoparticles or inhibitors.

The compounds and polymers to the present invention are useful as charge transport, semiconducting, electrically conducting, photoconducting or light emitting materials in optical, electrooptical, electronic, electroluminescent or photoluminescent components or devices. In these devices, the polymers of the present invention are typically applied as thin layers or films.

Thus, the present invention also provides the use of the semiconducting compound, polymer, polymers blend, formulation or layer in an electronic device. The formulation may be used as a high mobility semiconducting material in various devices and apparatus. The formulation may be used, for example, in the form of a semiconducting layer or film. Accordingly, in another aspect, the present invention provides a semiconducting layer for use in an electronic device, the layer comprising a compound, polymer, polymer blend or formulation according to the invention. The layer or film may be less than about 30 microns. For various electronic device applications, the thickness may be less than about 1 micron thick. The layer may be deposited, for example on a part of an electronic device, by any of the aforementioned solution coating or printing techniques.

The invention additionally provides an electronic device comprising a compound, polymer, polymer blend, formulation or organic semiconducting layer according to the present invention. Especially preferred devices are OFETs, TFTs, ICs, logic circuits, capacitors, RFID tags, OLEDs, OLETs, OPEDs, OPVs, OPDs, solar cells, laser diodes, photoconductors, photodetectors, electrophotographic devices, electrophotographic recording devices, organic memory devices, sensor devices, charge injection layers, Schottky diodes, planarising layers, antistatic films, conducting substrates and conducting patterns.

Especially preferred electronic device are OFETs, OLEDs and OPV devices, in particular bulk heterojunction (BHJ) OPV devices and OPD devices. In an OFET, for example, the active semiconductor channel between the drain and source may comprise the layer of the invention. As another example, in an OLED device, the charge (hole or electron) injection or transport layer may comprise the layer of the invention.

For use in OPV or OPD devices the polymer according to the present invention is preferably used in a formulation that comprises or contains, more preferably consists essentially of, very preferably exclusively of, a p-type (electron donor) semiconductor and an n-type (electron acceptor) semiconductor. The p-type semiconductor is constituted by a polymer according to the present invention. The n-type semiconductor can be an inorganic material such as zinc oxide (ZnOₓ), zinc tin oxide (ZTO), titan oxide (TiOₓ), molybdenum oxide (MoOₓ), nickel oxide (NiOₓ), or cadmium selenide (CdSe), or an organic material such as graphene or a fullerene or substituted fullerene, for example an indene-C₆₀-fullerene bisaduct like ICBA, or a (6,6)-phenyl-butyric acid methyl ester derivatized methano C₆₀ fullerene, also known as “PCBM-C₆₀” or “C₆₀PCBM”, as disclosed for example in G. Yu, J. Gao, J.C. Hummelen, F. Wudl, A. J. Heeger, Science 1995, Vol. 270, p. 1789 ff and having the structure shown below, or structural analogous compounds with e.g. a C₆₁ fullerene group, a C₇₀ fullerene group, or a C₇₁ fullerene group, or an organic polymer (see for example Coakley, K. M. and McGehee, M. D. Chem. Mater. 2004, 16, 4533).

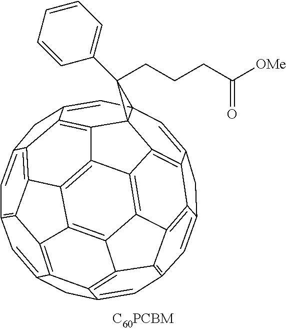 [SMILES: COC(=O)CCCC1(c2ccccc2)C23C=C4Cc5cc6cc7c8c9c(cc%10cc%11c%12c(c%13c%14c%15c(c4c5c4c6c8c(c%154)c4c9c%10c%12c%144)C%1321)C(=C3)C%11)C7]

Preferably the polymer according to the present invention is blended with an n-type semiconductor such as a fullerene or substituted fullerene, like for example PCBM-C₆₀, PCBM-C₇₀, PCBM-C₆₁, PCBM-C₇₁, bis-PCBM-C₆₁, bis-PCBM-C₇₁, ICBA (1′,1″,4′,4″-tetrahydro-di[1,4]methanonaphthaleno[1,2:2′,3′;56,60:2″,3″][5,6]fullerene-C60-lh), graphene, or a metal oxide, like for example, ZnOₓ, TiOₓ, ZTO, MoOₓ, NiOₓ, to form the active layer in an OPV or OPD device. The device preferably further comprises a first transparent or semi-transparent electrode on a transparent or semi-transparent substrate on one side of the active layer, and a second metallic or semi-transparent electrode on the other side of the active layer.

Further preferably the OPV or OPD device comprises, between the active layer and the first or second electrode, one or more additional buffer layers acting as hole transporting layer and/or electron blocking layer, which comprise a material such as metal oxide, like for example, ZTO, MoOₓ, NiOₓ a conjugated polymer electrolyte, like for example PEDOT:PSS, a conjugated polymer, like for example polytriarylamine (PTAA), an organic compound, like for example N,N′-diphenyl-N,N′-bis(1-naphthyl)(1,1′-biphenyl)-4,4′diamine (NPB), N,N′-diphenyl-N,N′-(3-methylphenyl)-1,1′-biphenyl-4,4′-diamine (TPD), or alternatively as hole blocking layer and/or electron transporting layer, which comprise a material such as metal oxide, like for example, ZnOₓ, TiOₓ, a salt, like for example LiF, NaF, CsF, a conjugated polymer electrolyte, like for example poly[3-(6-trimethylammoniumhexyl)thiophene], poly(9,9-bis(2-ethylhexyl)-fluorene]-b-poly[3-(6-trimethylammoniumhexyl)thiophene], or poly [(9,9-bis(3″-(N,N-dimethylamino)propyl)-2,7-fluorene)-alt-2,7-(9,9-dioctylfluorene)] or an organic compound, like for example tris(8-quinolinolato)-aluminium(III) (Alq₃), 4,7-diphenyl-1,10-phenanthroline.

In a blend or mixture of a polymer according to the present invention with a fullerene or modified fullerene, the ratio polymer:fullerene is preferably from 5:1 to 1:5 by weight, more preferably from 1:1 to 1:3 by weight, most preferably 1:1 to 1:2 by weight. A polymeric binder may also be included, from 5 to 95% by weight. Examples of binder include polystyrene (PS), polypropylene (PP) and polymethylmethacrylate (PMMA).

To produce thin layers in BHJ OPV devices the compounds, polymers, polymer blends or formulations of the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, dip coating, curtain coating, brush coating, slot dye coating or pad printing. For the fabrication of OPV devices and modules area printing method compatible with flexible substrates are preferred, for example slot dye coating, spray coating and the like.

Suitable solutions or formulations containing the blend or mixture of a polymer according to the present invention with a C₆₀ or C₇₀ fullerene or modified fullerene like PCBM must be prepared. In the preparation of formulations, suitable solvent must be selected to ensure full dissolution of both component, p-type and n-type and take into account the boundary conditions (for example rheological properties) introduced by the chosen printing method.

Organic solvents are generally used for this purpose. Typical solvents can be aromatic solvents, halogenated solvents or chlorinated solvents, including chlorinated aromatic solvents. Examples include, but are not limited to chlorobenzene, 1,2-dichlorobenzene, chloroform, 1,2-dichloroethane, dichloromethane, carbon tetrachloride, toluene, cyclohexanone, ethylacetate, tetrahydrofuran, anisole, morpholine, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, dimethylformamide, dimethylacetamide, dimethylsulfoxide, tetraline, decaline, indane, methyl benzoate, ethyl benzoate, mesitylene and combinations thereof.

The OPV device can for example be of any type known from the literature (see e.g. Waldauf et al., Appl. Phys. Lett., 2006, 89, 233517).

A first preferred OPV device according to the invention comprises the following layers (in the sequence from bottom to top):
optionally a substrate,
a high work function electrode, preferably comprising a metal oxide, like for example ITO, serving as anode,
an optional conducting polymer layer or hole transport layer, preferably comprising an organic polymer or polymer blend, for example of PEDOT:PSS (poly(3,4-ethylenedioxythiophene): poly(styrene-sulfonate), or TBD (N,N′-dyphenyl-N—N′-bis(3-methylphenyl)-1,1′biphenyl-4,4′-diamine) or NBD (N,N′-dyphenyl-N—N′-bis(1-napthylphenyl)-1,1′biphenyl-4,4′-diamine),
a layer, also referred to as “active layer”, comprising a p-type and an n-type organic semiconductor, which can exist for example as a p-type/n-type bilayer or as distinct p-type and n-type layers, or as blend or p-type and n-type semiconductor, forming a BHJ,
optionally a layer having electron transport properties, for example comprising LiF,
a low work function electrode, preferably comprising a metal like for example aluminum, serving as cathode,
wherein at least one of the electrodes, preferably the anode, is transparent to visible light, and
wherein the p-type semiconductor is a polymer according to the present invention.

A second preferred OPV device according to the invention is an inverted OPV device and comprises the following layers (in the sequence from bottom to top):
optionally a substrate,
a high work function metal or metal oxide electrode, comprising for example ITO, serving as cathode,
a layer having hole blocking properties, preferably comprising a metal oxide like TiOₓ or Znₓ,
an active layer comprising a p-type and an n-type organic semiconductor, situated between the electrodes, which can exist for example as a p-type/n-type bilayer or as distinct p-type and n-type layers, or as blend or p-type and n-type semiconductor, forming a BHJ,
an optional conducting polymer layer or hole transport layer, preferably comprising an organic polymer or polymer blend, for example of PEDOT:PSS or TBD or NBD,
an electrode comprising a high work function metal like for example silver, serving as anode,
wherein at least one of the electrodes, preferably the cathode, is transparent to visible light, and
wherein the p-type semiconductor is a polymer according to the present invention.

In the OPV devices of the present invention the p-type and n-type semiconductor materials are preferably selected from the materials, like the polymer/fullerene systems, as described above.

When the active layer is deposited on the substrate, it forms a BHJ that phase separates at nanoscale level. For discussion on nanoscale phase separation see Dennler et al, Proceedings of the IEEE, 2005, 93 (8), 1429 or Hoppe et al, Adv. Func. Mater, 2004, 14(10), 1005. An optional annealing step may be then necessary to optimize blend morpohology and consequently OPV device performance.

Another method to optimize device performance is to prepare formulations for the fabrication of OPV(BHJ) devices that may include high boiling point additives to promote phase separation in the right way. 1,8-Octanedithiol, 1,8-diiodooctane, nitrobenzene, chloronaphthalene, and other additives have been used to obtain high-efficiency solar cells. Examples are disclosed in J. Peet, et al, Nat. Mater., 2007, 6, 497 or Fréchet et al. J. Am. Chem. Soc., 2010, 132, 7595-7597.

The compounds, polymers, formulations and layers of the present invention are also suitable for use in an OFET as the semiconducting channel. Accordingly, the invention also provides an OFET comprising a gate electrode, an insulating (or gate insulator) layer, a source electrode, a drain electrode and an organic semiconducting channel connecting the source and drain electrodes, wherein the organic semiconducting channel comprises a compound, polymer, polymer blend, formulation or organic semiconducting layer according to the present invention. Other features of the OFET are well known to those skilled in the art.

OFETs where an OSC material is arranged as a thin film between a gate dielectric and a drain and a source electrode, are generally known, and are described for example in U.S. Pat. No. 5,892,244, U.S. Pat. No. 5,998,804, U.S. Pat. No. 6,723,394 and in the references cited in the background section. Due to the advantages, like low cost production using the solubility properties of the compounds according to the invention and thus the processability of large surfaces, preferred applications of these FETs are such as integrated circuitry, TFT displays and security applications.

The gate, source and drain electrodes and the insulating and semiconducting layer in the OFET device may be arranged in any sequence, provided that the source and drain electrode are separated from the gate electrode by the insulating layer, the gate electrode and the semiconductor layer both contact the insulating layer, and the source electrode and the drain electrode both contact the semiconducting layer.

An OFET device according to the present invention preferably comprises:
a source electrode,
a drain electrode,
a gate electrode,
a semiconducting layer,
one or more gate insulator layers,
optionally a substrate.

wherein the semiconductor layer preferably comprises a compound, polymer, polymer blend or formulation as described above and below.

The OFET device can be a top gate device or a bottom gate device. Suitable structures and manufacturing methods of an OFET device are known to the skilled in the art and are described in the literature, for example in US 2007/0102696 A1.

The gate insulator layer preferably comprises a fluoropolymer, like e.g. the commercially available Cytop 809M® or Cytop 107M® (from Asahi Glass). Preferably the gate insulator layer is deposited, e.g. by spin-coating, doctor blading, wire bar coating, spray or dip coating or other known methods, from a formulation comprising an insulator material and one or more solvents with one or more fluoro atoms (fluorosolvents), preferably a perfluorosolvent. A suitable perfluorosolvent is e.g. FC75® (available from Acros, catalogue number 12380). Other suitable fluoropolymers and fluorosolvents are known in prior art, like for example the perfluoropolymers Teflon AF® 1600 or 2400 (from DuPont) or Fluoropel® (from Cytonix) or the perfluorosolvent FC 43® (Acros, No. 12377). Especially preferred are organic dielectric materials having a low permittivity (or dielectric contant) from 1.0 to 5.0, very preferably from 1.8 to 4.0 (“low k materials”), as disclosed for example in US 2007/0102696 A1 or U.S. Pat. No. 7,095,044.

In security applications, OFETs and other devices with semiconducting materials according to the present invention, like transistors or diodes, can be used for RFID tags or security markings to authenticate and prevent counterfeiting of documents of value like banknotes, credit cards or ID cards, national ID documents, licenses or any product with monetary value, like stamps, tickets, shares, cheques etc.

Alternatively, the materials according to the invention can be used in OLEDs, e.g. as the active display material in a flat panel display applications, or as backlight of a flat panel display like e.g. a liquid crystal display. Common OLEDs are realized using multilayer structures. An emission layer is generally sandwiched between one or more electron-transport and/or hole-transport layers. By applying an electric voltage electrons and holes as charge carriers move towards the emission layer where their recombination leads to the excitation and hence luminescence of the lumophor units contained in the emission layer. The inventive compounds, materials and films may be employed in one or more of the charge transport layers and/or in the emission layer, corresponding to their electrical and/or optical properties. Furthermore their use within the emission layer is especially advantageous, if the compounds, materials and films according to the invention show electroluminescent properties themselves or comprise electroluminescent groups or compounds. The selection, characterization as well as the processing of suitable monomeric, oligomeric and polymeric compounds or materials for the use in OLEDs is generally known by a person skilled in the art, see, e.g., Müller et al, Synth. Metals, 2000, 111-112, 31-34, Alcala, J. Appl. Phys., 2000, 88, 7124-7128 and the literature cited therein.

According to another use, the materials according to this invention, especially those showing photoluminescent properties, may be employed as materials of light sources, e.g. in display devices, as described in EP 0 889 350 A1 or by C. Weder et al., Science, 1998, 279, 835-837.

A further aspect of the invention relates to both the oxidised and reduced form of the compounds according to this invention. Either loss or gain of electrons results in formation of a highly delocalised ionic form, which is of high conductivity. This can occur on exposure to common dopants. Suitable dopants and methods of doping are known to those skilled in the art, e.g. from EP 0 528 662, U.S. Pat. No. 5,198,153 or WO 96/21659.

The doping process typically implies treatment of the semiconductor material with an oxidating or reducing agent in a redox reaction to form delocalised ionic centres in the material, with the corresponding counterions derived from the applied dopants. Suitable doping methods comprise for example exposure to a doping vapor in the atmospheric pressure or at a reduced pressure, electrochemical doping in a solution containing a dopant, bringing a dopant into contact with the semiconductor material to be thermally diffused, and ion-implantation of the dopant into the semiconductor material.

When electrons are used as carriers, suitable dopants are for example halogens (e.g., I₂, Cl₂, Br₂, ICl, ICl₃, IBr and IF), Lewis acids (e.g., PF₅, AsF₅, SbF₅, BF₃, BCl₃, SbCl₅, BBr₃ and SO₃), protonic acids, organic acids, or amino acids (e.g., HF, HCl, HNO₃, H₂SO₄, HClO₄, FSO₃H and ClSO₃H), transition metal compounds (e.g., FeCl₃, FeOCl, Fe(ClO₄)₃, Fe(4-CH₃C₆H₄SO₃)₃, TiCl₄, ZrCl₄, HfCl₄, NbF₅, NbCl₅, TaCl₅, MoF₅, MoCl₅, WF₅, WCl₆, UF₆ and LnCl₃ (wherein Ln is a lanthanoid), anions (e.g., Cl⁻, Br⁻, I⁻, I₃⁻, HSO₄⁻, SO₄²⁻, NO₃⁻, ClO₄⁻, BF₄⁻, PF₆⁻, AsF₆⁻, SbF₆⁻, FeCl₄⁻, Fe(CN)₆³⁻, and anions of various sulfonic acids, such as aryl-SO₃⁻). When holes are used as carriers, examples of dopants are cations (e.g., H⁺, Li⁺, Na⁺, K⁺, Rb⁺ and Cs⁺), alkali metals (e.g., Li, Na, K, Rb, and Cs), alkaline-earth metals (e.g., Ca, Sr, and Ba), O₂, XeOF₄, (NO₂⁺) (SbF₆⁻), (NO₂⁺) (SbCl₆⁻), (NO₂⁺) (BF₄⁻), AgClO₄, H₂IrCl₆, La(NO₃)₃.6H₂O, FSO₂OOSO₂F, Eu, acetylcholine, R₄N⁺, (R is an alkyl group), R₄P⁺ (R is an alkyl group), R₆As⁺ (R is an alkyl group), and R₃S⁺ (R is an alkyl group).

The conducting form of the compounds of the present invention can be used as an organic “metal” in applications including, but not limited to, charge injection layers and ITO planarising layers in OLED applications, films for flat panel displays and touch screens, antistatic films, printed conductive substrates, patterns or tracts in electronic applications such as printed circuit boards and condensers.

The compounds and formulations according to the present invention may also be suitable for use in organic plasmon-emitting diodes (OPEDs), as described for example in Koller et al., Nat. Photonics, 2008, 2, 684.

According to another use, the materials according to the present invention can be used alone or together with other materials in or as alignment layers in LCD or OLED devices, as described for example in US 2003/0021913. The use of charge transport compounds according to the present invention can increase the electrical conductivity of the alignment layer. When used in an LCD, this increased electrical conductivity can reduce adverse residual dc effects in the switchable LCD cell and suppress image sticking or, for example in ferroelectric LCDs, reduce the residual charge produced by the switching of the spontaneous polarisation charge of the ferroelectric LCs. When used in an OLED device comprising a light emitting material provided onto the alignment layer, this increased electrical conductivity can enhance the electroluminescence of the light emitting material. The compounds or materials according to the present invention having mesogenic or liquid crystalline properties can form oriented anisotropic films as described above, which are especially useful as alignment layers to induce or enhance alignment in a liquid crystal medium provided onto said anisotropic film. The materials according to the present invention may also be combined with photoisomerisable compounds and/or chromophores for use in or as photoalignment layers, as described in US 2003/0021913 A1.

According to another use the materials according to the present invention, especially their water-soluble derivatives (for example with polar or ionic side groups) or ionically doped forms, can be employed as chemical sensors or materials for detecting and discriminating DNA sequences. Such uses are described for example in L. Chen, D. W. McBranch, H. Wang, R. Helgeson, F. Wudl and D. G. Whitten, Proc. Natl. Acad. Sci. U.S.A., 1999, 96, 12287; D. Wang, X. Gong, P. S. Heeger, F. Rininsland, G. C. Bazan and A. J. Heeger, Proc. Natl. Acad. Sci. U.S.A., 2002, 99, 49; N. DiCesare, M. R. Pinot, K. S. Schanze and J. R. Lakowicz, Langmuir, 2002, 18, 7785; D. T. McQuade, A. E. Pullen, T. M. Swager, Chem. Rev., 2000, 100, 2537.

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

It will be appreciated that many of the features described above, particularly of the preferred embodiments, are inventive in their own right and not just as part of an embodiment of the present invention. Independent protection may be sought for these features in addition to or alternative to any invention presently claimed.

Above and below, unless stated otherwise percentages are percent by weight and temperatures are given in degrees Celsius. The values of the dielectric constant c (“permittivity”) refer to values taken at 20° C. and 1,000 Hz.

The invention will now be described in more detail by reference to the following examples, which are illustrative only and do not limit the scope of the invention.

### Example 1

### 1-Methyl-[1,5]naphthyridin-1-ium

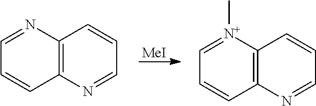

To a solution of [1,5]naphthyridine (5.0 g, 38.42 mmol) in toluene (100 cm³) is added iodomethane (7.2 cm³, 115.25 mmol), the reaction mixture is heated at 110° C. for 18 hours, and then cooled to 22° C. The resultant solids are collected by filtration and washed with further toluene to yield a yellow solid (5.6 g, 100%). ¹H NMR (300 MHz, D₂O) 9.34 (1H, d, ArH, J=5.8), 9.28 (1H, dd, ArH, J=4.3, 1.2), 9.16 (1H, d, ArH, J=8.9), 8.89 (1H, d, ArH, J=9.2), 8.28 (1H, dd, ArH, J=8.8, 5.8), 8.19 (1H, dd, ArH, J=9.1, 4.3), 4.68 (3H, s, CH₃).

### 1-Methyl-1H-[1,5]naphthyridin-2-one

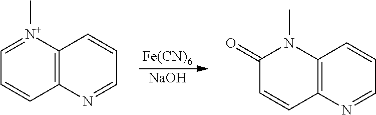

To a stirred solution of 1-methyl-[1,5]naphthyridin-1-ium (5.6 g, 38.57 mmol) in water (16 cm³) at 0° C. is added sodium hydroxide (6.2 g, 154.29 mmol) in water (16 cm³) and then potassium ferricyanide (25.4 g, 77.15 mmol) in water (16 cm³) dropwise. The reaction mixture is stirred at 0° C. for 1 hour and then at 22° C. for a further 2 hours. The reaction mixture is then extracted with chloroform, the organic phases combined and dried over magnesium sulphate before the solvent is removed in vacuo. The crude material is then purified by column chromatography (eluent: chloroform:methanol, 9:1) to yield the product as an orange solid (3.3 g, 54%). ¹H NMR (300 MHz, CDCl₃) 8.54 (1H, dd, ArH, J=4.4, 1.3), 7.88 (1H, d, ArH, J=9.8), 7.67 (1H, d, ArH, J=8.6), 7.46 (1H, dd, ArH, J=8.6, 4.4), 6.92 (1H, d, ArH, J=9.8), 3.68 (3H, s, CH₃).

### 1,5-Dimethyl-6-oxo-5,6-dihydro-[1,5]naphthyridin-1-ium

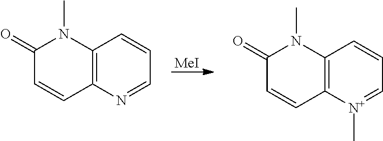

To a solution of 1-methyl-1H-[1,5]naphthyridin-2-one (0.26 g, 1.59 mmol) in toluene (5 cm³) is added iodomethane (0.11 cm³, 1.75 mmol), the reaction mixture is heated at 110° C. for 18 hours, then cooled to 22° C. The resultant solids are collected by filtration and washed with further toluene to yield an orange solid (0.28 g, 100%). ¹H NMR (300 MHz, D₂O) 8.80 (1H, d, ArH, J=5.9), 8.70 (1H, d, ArH, J=9.1), 8.41 (1H, d, ArH, J=10.8), 8.10 (1H, dd, ArH, J=9.1, 5.9), 7.30 (1H, d, ArH, J=10.3), 4.52 (3H, s, CH₃), 3.78 (3H, s, CH₃).

### 1,5-Dimethyl-1,5-dihydro-[1,5]naphthyridine-2,6-dione

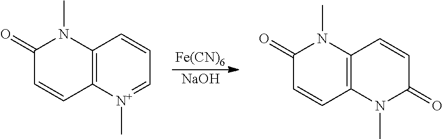

To a stirred solution of 1,5-dimethyl-6-oxo-5,6-dihydro-[1,5]naphthyridin-1-ium (0.37 g, 2.08 mmol) in water (4 cm³) at 0° C. is added sodium hydroxide (0.33 g, 8.33 mmol) in water (4 cm³) and then potassium ferricyanide (1.37 g, 4.17 mmol) in water (4 cm³) dropwise. The reaction mixture is stirred at 0° C. for 1 hour and then at 22° C. for a further 2 hours.

The reaction mixture is then extracted with chloroform, the organic phases combined and dried over magnesium sulphate before the solvent is removed in vacuo. The crude material is then purified by recrystallisation from acetonitrile/tetrahydrofuran to yield the product as orange needles (0.25 g, 63%). ¹H NMR (300 MHz, CDCl₃) 7.60 (2H, d, ArH, J=10.0), 6.87 (2H, d, ArH, J=10.0), 3.72 (6H, s, CH₃).

### 3,7-Dibromo-1,5-dimethyl-1,5-dihydro-[1,5]naphthyridine-2,6-dione

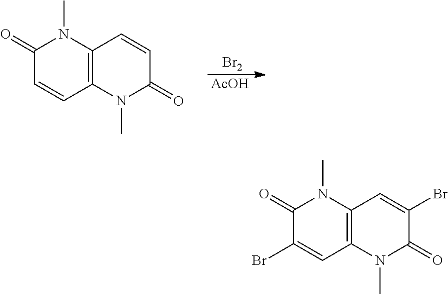 [SMILES: "AcOH" ; Cn1c(=O)ccc2c1ccc(=O)n2C ; "Br2" ; Cn1c(=O)c(Br)cc2c1cc(Br)c(=O)n2C]

To a solution of 1,5-dimethyl-1,5-dihydro-[1,5]naphthyridine-2,6-dione (0.25 g, 1.31 mmol) in acetic acid (5 cm³) is added dropwise bromine (0.14 cm³, 2.76 mmol) in acetic acid (1 cm³), and the mixture stirred at 22° C. in the dark for 18 hours. The reaction mixture is then quenched with water and the resultant precipitate collected by filtration to yield the product as an orange solid (0.46 g, 100%).

### 3,7-Bis-[4-(2-ethyl-hexyl)-thiophen-2-yl]-1,5-dimethyl-1,5-dihydro-[1,5]naphthyridine-2,6-dione

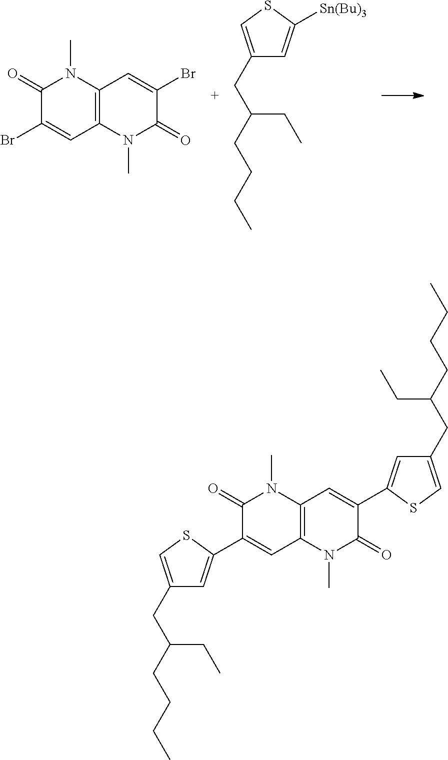

To a degassed mixture of 3,7-dibromo-1,5-dimethyl-1,5-dihydro-[1,5]naphthyridine-2,6-dione (1.30 g, 3.74 mmol) and tributyl-[4-(2-ethyl-hexyl)-thiophen-2-yl]-stannane (5.44 g, 11.21 mmol) in DMF (75 cm³) is added PdCl₂(PPh₃)₂ (131 mg, 0.19 mmol) and the mixture further degassed for 5 minutes. The mixture is then heated at 100° C. for 17 hours and stirred at 22° C. for 4 days before water is added and the product extracted with dichloromethane. The combined organic extracts are dried over anhydrous magnesium sulfate, filtered and the solvent removed in vacuo to give an orange oil. The crude product is purified by column chromatography (eluent: chloroform:methanol, 99:1; 10% potassium carbonate in silica) to give a red/orange oily solid. The solids are then redissolved in dichloromethane and washed with water, dried over magnesium sulfate and the solvent removed in vacuo to yield the product as a red solid (0.70 g, 32%). ¹H NMR (300 MHz, CDCl₃) 7.93 (2H, s, ArH), 7.61 (2H, d, ArH, J=1.3), 7.08 (2H, d, ArH, J=1.1), 3.90 (6H, s, CH₃), 2.56 (4H, d, CH₂, J=7.0), 1.66-1.53 (2H, m, CH), 1.34-1.23 (16H, m, CH₂), 0.87 (12H, t, CH₃, J=7.35).

### 3,7-Bis-[5-bromo-4-(2-ethyl-hexyl)-thiophen-2-yl]-1,5-dimethyl-1,5-dihydro-[1,5]naphthyridine-2,6-dione

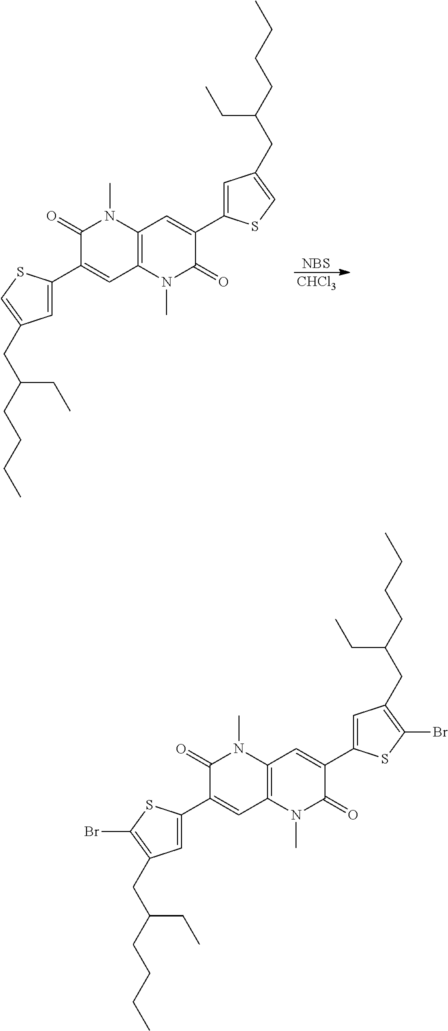

To a solution of 3,7-bis-[4-(2-ethyl-hexyl)-thiophen-2-yl]-1,5-dimethyl-1,5-dihydro-[1,5]naphthyridine-2,6-dione (157 mg, 0.271 mmol) in chloroform (15 cm³) is added 1-bromo-pyrrolidine-2,5-dione (96.5 mg, 0.54 mmol), the reaction mixture is stirred at 22° C. in the dark for 1 hour. The solvent is removed in vacuo and the crude solid recrystallised from acetonitrile/tetrahydrofuran to yield the product as a red solid (0.10 g, 50%). ¹H NMR (300 MHz, CDCl₃) 7.82 (2H, s, ArH), 7.34 (2H, d, ArH, J=1.3), 3.87 (6H, s, CH₃), 2.50 (4H, d, CH₂, J=7.3), 1.67-1.59 (2H, m, CH), 1.35-1.23 (16H, m, CH₂), 0.90-0.86 (12H, m, CH₃).

### Poly-3,7-bis-[4-(2-ethyl-hexyl)-thiophen-2-yl]-1,5-dimethyl-1,5-dihydro-[1,5]naphthyridine-2,6-dione-thiophene (Polymer P1)

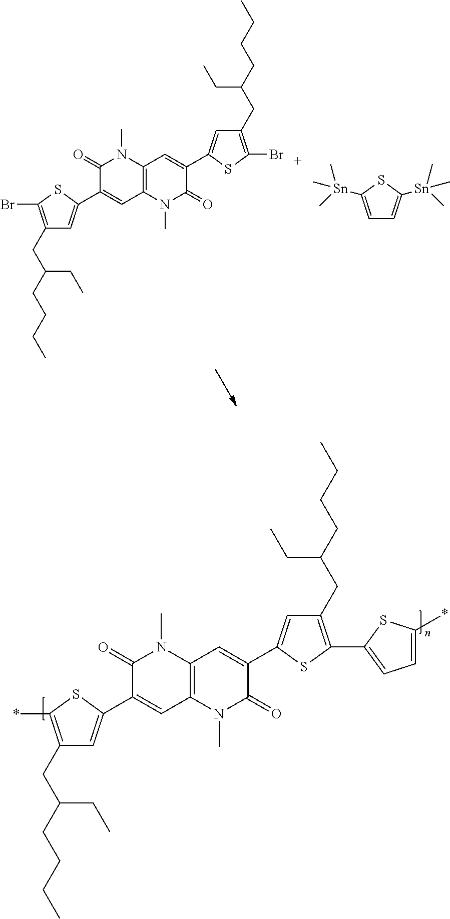

To a dry microwave vial is added 3,7-bis-[5-bromo-4-(2-ethyl-hexyl)-thiophen-2-yl]-1,5-dimethyl-1,5-dihydro-[1,5]naphthyridine-2,6-dione (128.1 mg, 0.17 mmol), 2,5-bis-trimethylstannanyl-thiophene (71.3 mg, 0.17 mmol) and Pd(PPh₃)₂Cl₂ (3.7 mg, 0.005 mmol), the vial is evacuated and nitrogen purged (×3) and then degassed toluene (4 cm³) and degassed N,N-dimethylformamide (1 cm³) are added. The solution is degassed for a further 1 hour before heating to 110° C. for 2 hours, then further heated in a microwave reactor (Biotage Initiator) at 140° C. for 1 minute, 160° C. for 1 minute and 170° C. for 30 minutes. The reaction mixture is precipitated into methanol and collected by filtration to yield a dark blue polymer (77 mg, 67%).

GPC, 1,2,4-trichlorobenzene (140° C.): Mₙ=4.6 kg·mol⁻¹, M_{w}=7.4 kg·mol⁻¹, PDI=1.6.

### Example 2

### Poly-3,7-bis-[4-(2-ethyl-hexyl)-thiophen-2-yl]-1,5-dimethyl-1,5-dihydro-[1,5]naphthyridine-2,6-dione-4,8-bis-(2-ethyl-hexyloxy)-benzo[1,2-b;4,5-b]dithiophene (Polymer P2)

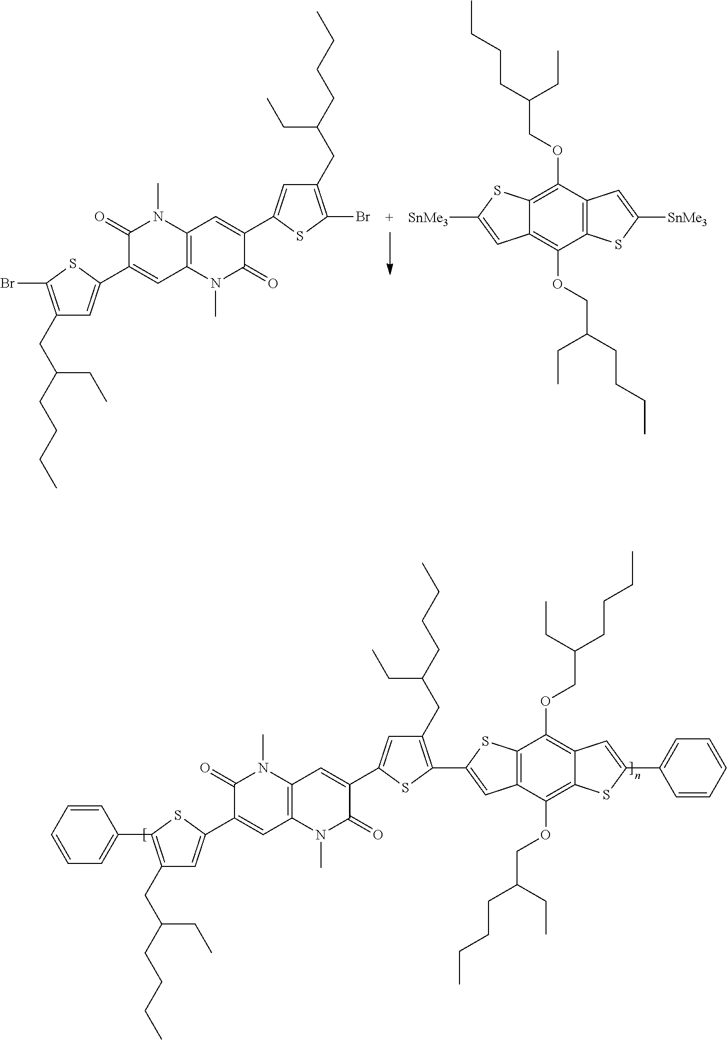

To a dry microwave vial is added 3,7-bis-[5-bromo-4-(2-ethyl-hexyl)-thiophen-2-yl]-1,5-dimethyl-1,5-dihydro-[1,5]naphthyridine-2,6-dione (500 mg, 0.68 mmol), 4,8-bis-(2-ethyl-hexyloxy)-2,6-bis-trimethylstannanyl-benzo[1,2-b;4,5-b′]dithiophene (524 mg, 0.68 mmol), Pd₂(dba)₃ (12.4 mg, 0.014 mmol) and tri-o-tolyl phosphine (16.5 mg, 0.054 mmol), the vial is evacuated and nitrogen purged (×3) and then degassed toluene (5.5 cm³) and degassed N,N-dimethylformamide (1.3 cm³) are added. The solution is degassed for a further 30 minutes before heating to 110° C. for 10 minutes. The reaction mixture is then end-capped with tributylphenyl-stannane (0.22 ml, 0.68 mmol), heated to 110° C. for 1 hour, and then bromobenzene (0.11 ml, 1.02 mmol) is added and the reaction mixture heated to 110° C. for a further 1 hour. The reaction mixture is precipitated into methanol and collected by filtration, and then purified via sequential Soxhlet extraction with acetone, petroleum ether 40-60, cyclohexane and chloroform. The chloroform fraction is then precipitated into methanol to yield a dark blue polymer (0.53 g, 76%).

GPC, chlorobenzene (50° C.): Mₙ=6.5 kg·mol⁻¹, M_{w}=16.3 kg·mol⁻¹, PDI=2.5.

### Example 3

Bulk heterojunction organic photovoltaic devices (OPVs) for Polymer P2

OPV devices are fabricated on ITO-glass substrates (13Ω/), purchased from Zencatec. Substrates are subjected to a conventional photolithography process to define the bottom electrodes (anodes) before cleaning using common solvents (acetone, IPA, DI water) in an ultrasonic bath.

A conducting polymer poly(ethylene dioxythiophene) doped with poly(styrene sulfonic acid) [Clevios VPAI 4083 (H.C. Starck)] is mixed in a 1:1 ratio with DI-water. This solution is sonicated for 20 minutes to ensure proper mixing and filtered using a 0.2 μm filter before spin coating to a thickness of 20 nm. Substrates are exposed to a UV-ozone treatment prior to the spin-coating process to ensure good wetting properties. Films are then annealed at 130° C. for 30 minutes in an inert atmosphere.

Photoactive material solutions are prepared at the concentration and components ratio stated on the examples, and stirred overnight. Thin films are either spin coated or blade coated in an inert atmosphere to achieve thicknesses between 100 and 200 nm, measured using a profilemeter. A short drying period follows to ensure removal of excess solvent. Typically, spin coated films are dried at 23° C. for 10 minutes. Blade coated films are dried at 70° C. for 3 minutes on the hotplate.

As the last step of the device fabrication, Calcium (30 nm)/Al (200 nm) cathodes are thermally evaporated through a shadow mask to define cells. Samples are measured at 23° C. using a Solar Simulator from Newport Ltd (model 91160) as a light source, calibrated to 1 sun using a Si reference cell.

The following device performance for polymer P2 is obtained as described in table 1.

TABLE 1
Average open circuit potential (V_{oc}), current density
(J_{SC}), fill factor (FF), power conversion efficiency
(PCE) and best power conversion efficiency for specific
ratios of polymer P2 and PCBM-C₆₀.

| Ratio | | | | | |
|---|---|---|---|---|---|
| Polymer | conc. | Voc | Jsc | FF | PCE |
| P2:PCBM | mg · cm⁻³ | mV | mA · cm⁻² | % | % |
| 1.0:1.0 | 30 | 680 | −7.93 | 47.8 | 2.57 |
| 1.0:1.5 | 30 | 680 | −8.19 | 49.4 | 2.75 |
| 1.0:2.0 | 30 | 664 | −5.66 | 49.6 | 1.86 |
| 1.0:3.0 | 30 | 671 | −6.90 | 49.5 | 2.29 |

### Example 4

### Poly-3,7-bis-[4-(2-ethyl-hexyl)-thiophen-2-yl]-1,5-dimethyl-1,5-dihydro-[1,5]naphthyridine-2,6-dione-4,8-bis-(1-octyl-nonyloxy)-benzo[1,2-b;4,5-b′]dithiophene (Polymer P3)

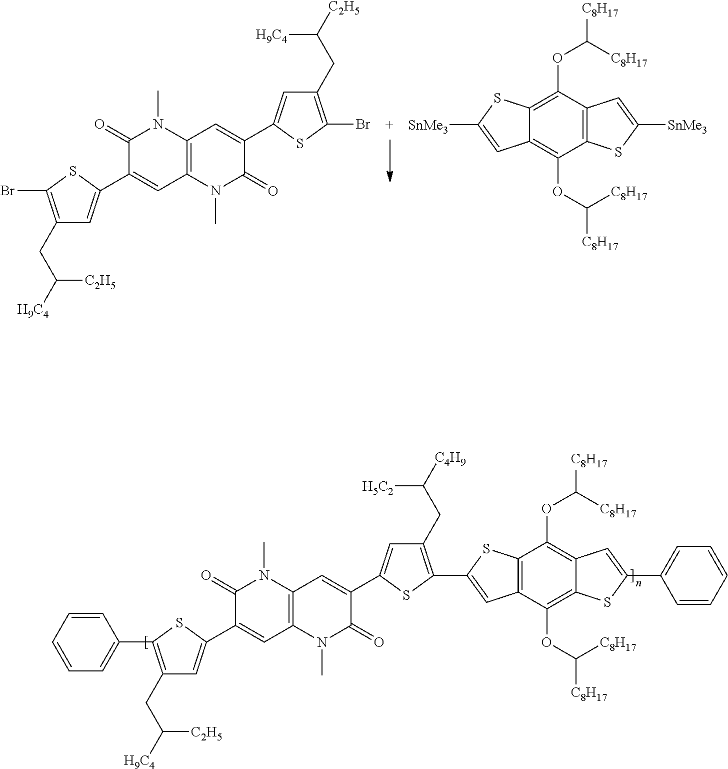

To a dry microwave vial is added 3,7-bis-[5-bromo-4-(2-ethyl-hexyl)-thiophen-2-yl]-1,5-dimethyl-1,5-dihydro-[1,5]naphthyridine-2,6-dione (422.5 mg, 0.57 mmol), 4,8-bis-(1-octyl-nonyloxy)-2,6-bis-trimethylstannanyl-benzo[1,2-b;4,5-b′]dithiophene (588 mg, 0.57 mmol), Pd₂(dba)₃ (10.5 mg, 0.011 mmol) and tri-o-tolyl phosphine (14.0 mg, 0.046 mmol), the vial is evacuated and nitrogen purged (×3) and then degassed toluene (4.6 cm³) and degassed N,N-dimethylformamide (1.2 cm³) are added. The solution is degassed for a further 30 minutes before heating to 110° C. for 3 hours and 20 minutes. The reaction mixture is then end-capped with tributylphenyl-stannane (0.19 ml, 0.57 mmol), heated to 110° C. for 1 hour, and then bromobenzene (0.19 ml, 0.86 mmol) is added and the reaction mixture heated to 110° C. for a further 1 hour. The reaction mixture is precipitated into methanol and collected by filtration and purified via sequential Soxhlet extraction with acetone, petroleum ether 40-60 and cyclohexane. The cyclohexane fraction is then reduced in vacuo and redissolved in chloroform, then precipitated into methanol to yield a black polymer (0.25 g, 34%).

GPC, chlorobenzene (50° C.): Mₙ=19.4 kg·mol⁻¹, M_{w}=38.0 kg·mol⁻¹, PDI=1.96.

### Example 5

OPV devices were built as previously described for polymer P2.

The following device performance for polymer P3 is obtained as described in table 2.

TABLE 2
Average open circuit potential (V_{oc}), current density
(J_{SC}), fill factor (FF), power conversion efficiency
(PCE) and best power conversion efficiency for specific
ratios of polymer P3 and PCBM-C₆₀.

| Ratio | | | | | |
|---|---|---|---|---|---|
| Polymer | conc. | Voc | Jsc | FF | PCE |
| P3:PCBM | mg · cm⁻³ | mV | mA · cm⁻² | % | % |
| 1.0:1.0 | 30 | 775 | −1.19 | 52.3 | 0.48 |
| 1.0:1.5 | 30 | 765 | −1.86 | 58.2 | 0.83 |
| 1.0:2.0 | 20 | 765 | −2.24 | 61.8 | 1.06 |
| 1.0:3.0 | 30 | 757 | −1.31 | 58.0 | 0.57 |

### Example 6

### Poly-3,7-bis-[4-(2-ethyl-hexyl)-thiophen-2-yl]-1,5-dimethyl-1,5-dihydro-[1,5]naphthyridine-2,6-dione-4,8-bis-(1-dodecyl-tridecyloxy)-benzo[1,2-b;4,5-b′]dithiophene (Polymer P4)

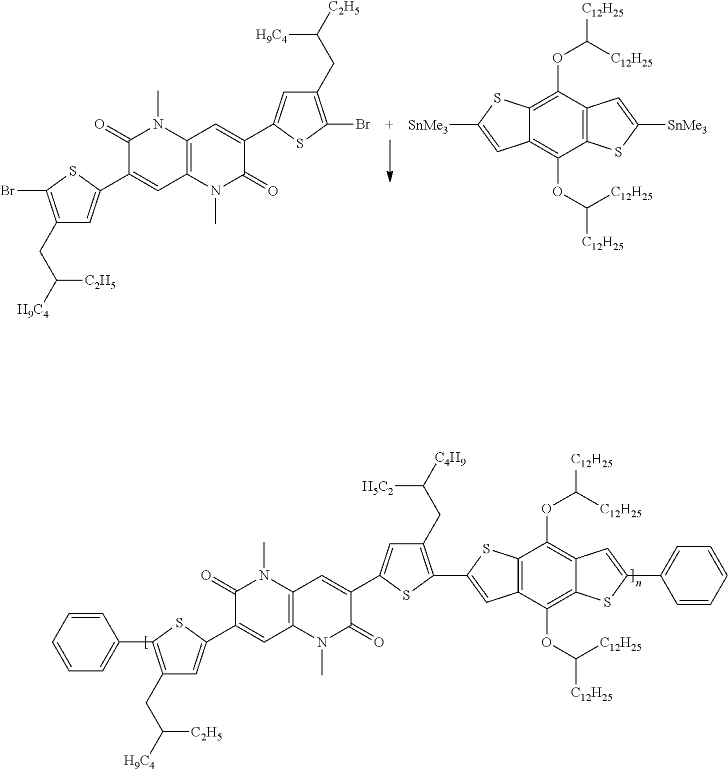

To a dry microwave vial is added 3,7-bis-[5-bromo-4-(2-ethyl-hexyl)-thiophen-2-yl]-1,5-dimethyl-1,5-dihydro-[1,5]naphthyridine-2,6-dione (294.8 mg, 0.40 mmol), 4,8-bis-(1-dodecyl-tridecyloxy)-2,6-bis-trimethylstannanyl-benzo[1,2-b;4,5-b′]dithiophene (499.9 mg, 0.40 mmol), Pd₂(dba)₃ (7.3 mg, 0.008 mmol) and tri-o-tolyl phosphine (9.7 mg, 0.032 mmol), the vial is evacuated and nitrogen purged (×3) and then degassed toluene (4.0 cm³) and degassed N,N-dimethylformamide (1.0 cm³) is added. The solution is degassed for a further 30 minutes before heating to 110° C. for 1 day. The reaction mixture is then end-capped with tributylphenyl-stannane (0.13 ml, 0.40 mmol), heated to 110° C. for 1 hour, and then bromobenzene (0.06 ml, 0.60 mmol) is added and the reaction mixture heated to 110° C. for a further 1 hour. The reaction mixture is precipitated into methanol and collected by filtration and purified via sequential Soxhlet extraction with acetone and petroleum ether 40-60. The petroleum ether 40-60 fraction is then reduced in vacuo and redissolved in chloroform, then precipitated into methanol to yield a black polymer (0.36 g, 60%).

GPC, chlorobenzene (50° C.): Mₙ=15.0 kg·mol⁻¹, M_{w}=28.9 kg·mol⁻¹, PDI=1.93.

### Example 7

### 2-Bromo-6-methoxy-pyridin-3-ylamine

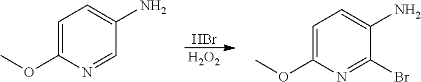

To a solution of 6-methoxy-pyridin-3-ylamine (44.6 g, 360 mmol) in 48% HBr in water (606 cm³) at 5° C. is added 30% hydrogen peroxide in water (45 ml, 396 mmol) dropwise over 30 minutes. The reaction mixture is allowed to warm to 22° C. with stirring over 17 hours, and quenched with aqueous sodium hydroxide. The aqueous phase is extracted with ethyl acetate and the organics combined before washing with brine and drying over magnesium sulphate. The solvent is removed in vacuo to yield the product as a brown oil (64.4 g, 88%). ¹H NMR (300 MHz, DMSO-d) 7.19 (1H, d, ArH, J=8.5), 6.65 (1H, d, ArH, J=8.6), 4.93 (1H, br. s, NH₂), 3.71 (3H, s, CH₃).

### 6-Methoxy-1H-[1,5]naphthyridin-2-one

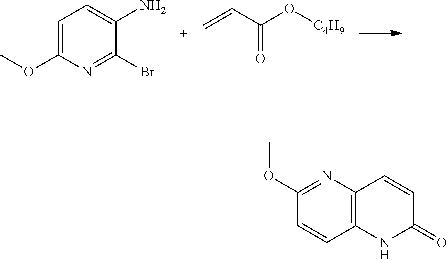

To a solution of 2-bromo-6-methoxy-pyridin-3-ylamine (66.4 g, 327 mmol) in cumene (350 cm³) is added dicyclohexylmethylamine (210 ml, 982 mmol) and the reaction mixture is degassed for 30 minutes. Acrylic acid butyl ester (56 ml, 393 mmol), palladium acetate (1.47 g, 6.55 mmol) and tributylphosphine tetrafluoroborate (3.80 g, 13.1 mmol) is added and the reaction mixture evacuated and nitrogen purged (×3). The reaction mixture is then heated at 150° C. for 17 hours, allowed to warm to 22° C. and quenched with aqueous sodium hydroxide. The aqueous is extracted with diethyl ether and the organics separated, the aqueous phase is then acidified to pH 2 with dilute HCl, the resultant precipitate collected by filtration and dried to yield the product as a cream solid (57.1 g, 99%). ¹H NMR (300 MHz, DMSO-d) 7.80 (1H, d, ArH, J=9.7), 7.68 (1H, d, ArH, J=8.9), 7.03 (1H, d, ArH, J=9.0), 6.68 (1H, d, ArH, J=9.7), 3.87 (3H, s, CH₃).

### 1,5-Dihydro-[1,5]naphthyridine-2,6-dione

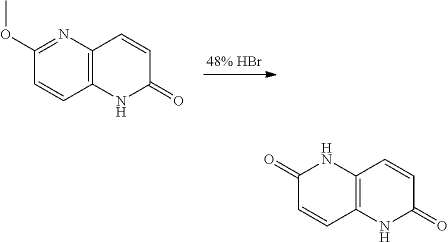

To a solution of 48% HBr in water (45 cm³) is added 6-methoxy-1H-[1,5]naphthyridin-2-one (1.90 g, 10.8 mmol) and the reaction mixture is heated to reflux for 2.5 hours. The reaction mixture is then cooled to 22° C. and adjusted to pH 7 with aqueous sodium carbonate, the resulting suspension is then cooled to 0° C. and the solids collected by filtration to yield the product as a beige solid (1.53 g, 87%). ¹H NMR (300 MHz, TFA-d) 7.47 (1H, d, ArH, J=9.8), 7.33 (1H, d, ArH, J=9.0), 6.71 (1H, d, ArH, J=8.9), 6.35 (1H, d, ArH, J=9.7), 3.55 (3H, s, CH₃).

### 1,5-Didodecyl-1,5-dihydro-[1,5]naphthyridine-2,6-dione

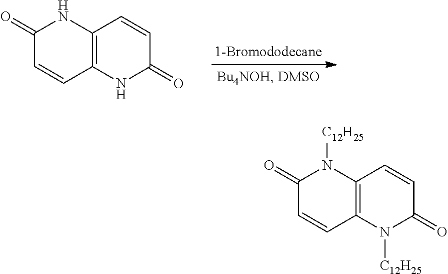

To a solution of 40% tetrabutylammonium hydroxide in water (250 cm³, 370.0) is added 1,5-dihydro-[1,5]naphthyridine-2,6-dione (5.0 g, 30.8 mmol), 1-bromododecane (50 cm³, 208.2 mmol) and DMSO (60 cm³), the reaction mixture is stirred at 65° C. for 3 days. The reaction is precipitated with aqueous ammonium chloride and the solids collected by filtration. The crude solid is then washed with hot petroleum ether 40-60/ethyl acetate (1:1) and the solids collected by filtration to yield the product as a yellow solid (1.7 g, 11%). ¹H NMR (300 MHz, CDCl₃) 7.55 (2H, d, ArH, J=10.1), 6.86 (2H, d, ArH, J=10.1), 4.22 (4H, t, CH₂, J=7.9), 1.70 (4H, m, CH₂), 1.47-1.17 (36H, m, CH₂), 0.88 (6H, t, CH₃, J=6.9).

### 3,7-Dibromo-1,5-didodecyl-1,5-dihydro-[1,5]naphthyridine-2,6-dione

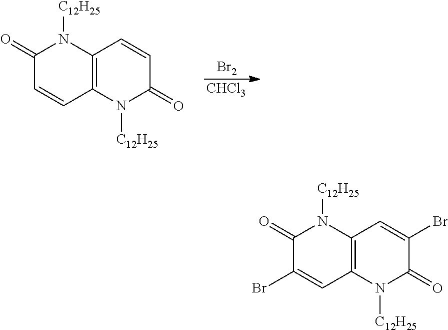

To a solution of 1,5-didodecyl-1,5-dihydro-[1,5]naphthyridine-2,6-dione (1.63 g, 3.26 mmol) in chloroform (60 cm³) is added bromine (0.37 ml, 7.17 mmol), the reaction mixture is stirred at reflux in the dark for 17 hours. The reaction is precipitated with methanol and the solids collected by filtration to yield a yellow solid. The crude solid is then purified by recrystallisation from methanol/tetrahydrofuran to yield the product as bright yellow needles (0.82 g, 38%). ¹H NMR (300 MHz, CDCl₃) 7.95 (2H, s, ArH), 4.26 (4H, t, CH₂, J=7.9), 1.73 (4H, tt, CH₂, J=7.5), 1.51-1.27 (36H, m, CH₂), 0.89 (6H, t, CH₃, J=7.0).

### Example 8

### 1,5-Dihexadecyl-1,5-dihydro-[1,5]naphthyridine-2,6-dione

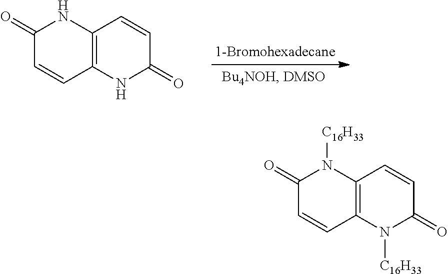

To a solution of 40% tetrabutylammonium hydroxide in water (100 cm³, 150 mmol) is added 1,5-dihydro-[1,5]naphthyridine-2,6-dione (2.0 g, 12.3 mmol), 1-bromohexadecane (18.8 cm³, 61.7 mmol) and DMSO (25 cm³), the reaction mixture is stirred at 65° C. for 3 days. The reaction is precipitated with aqueous ammonium chloride and the solids collected by filtration. The crude solid is then purified by column chromatography (eluent: dichloromethane:methanol, 99:1) and recrystallised from methanol/dichloromethane. Further purification by column chromatography (eluent: ethyl acetate:petroleum ether 40-60, 1:1, then eluent: dichloromethane:methanol, 99:1) yields the product as a yellow solid (0.52 g, 7%). ¹H NMR (300 MHz, CDCl₃) 7.55 (2H, d, ArH, J=10.1), 6.87 (2H, d, ArH, J=10.0), 4.23 (4H, t, CH₂, J=7.9), 1.70 (4H, m, CH₂), 1.45-1.26 (52H, m, CH₂), 0.88 (6H, t, CH₃, J=6.9).

### 3,7-Dibromo-1,5-dihexadecyl-1,5-dihydro-[1,5]naphthyridine-2,6-dione

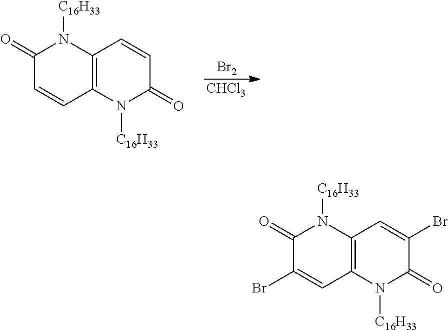

To a solution of 1,5-dihexadecyl-1,5-dihydro-[1,5]naphthyridine-2,6-dione (0.50 g, 0.82 mmol) in chloroform (30 cm³) is added bromine (0.11 ml, 2.05 mmol), the reaction mixture is stirred at reflux in the dark for 17 hours. The reaction is precipitated with methanol and the solids collected by filtration to yield a yellow solid. The crude solid is then purified by recrystallisation from methanol/tetrahydrofuran to yield the product as bright yellow needles (0.59 g, 94%). ¹H NMR (300 MHz, CDCl₃) 7.95 (2H, s, ArH), 4.26 (4H, t, CH₂, J=7.9), 1.72 (4H, m, CH₂), 1.56-1.26 (52H, m, CH₂), 0.89 (6H, t, CH₃, J=6.9).

### Example 9

### Polymer P5

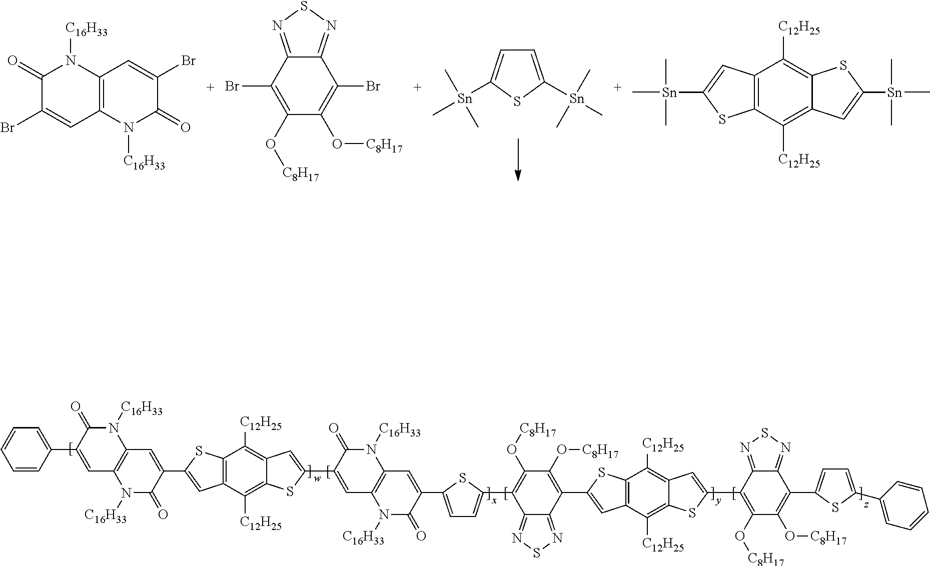

To a dry microwave vial is added 3,7-dibromo-1,5-dihexadecyl-1,5-dihydro-[1,5]naphthyridine-2,6-dione (62 mg, 0.08 mmol), 4,7-dibromo-5,6-bis-octyloxy-benzo[1,2,5]thiadiazole (396 mg, 0.72 mmol), 2,5-bis-trimethylstannanyl-thiophene (164 mg, 0.40 mmol), 4,8-didodecyl-2,6-bis-trimethylstannanyl-benzo[1,2-b;4,5-b′]dithiophene (341 mg, 0.40 mmol), Pd₂(dba)₃ (14.7 mg, 0.016 mmol) and tri-o-tolyl phosphine (19.5 mg, 0.064 mmol), the vial is evacuated and nitrogen purged (×3) and then degassed chlorobenzene (5.0 cm³) is added. The solution is degassed for a further 15 minutes before heating to 140° C. for 2 hours. The reaction mixture is then end-capped with tributylphenyl-stannane (0.26 ml, 0.80 mmol), heated to 140° C. for 1 hour, and then bromobenzene (0.13 ml, 1.20 mmol) is added and the reaction mixture heated to 140° C. for a further 1 hour. The reaction mixture is precipitated into methanol and collected by filtration and purified via sequential Soxhlet extraction with acetone, petroleum ether 40-60 and cyclohexane. The cyclohexane fraction is then reduced in vacuo and redissolved in chloroform, then precipitated into methanol to yield a black polymer (0.56 g, 98%).

GPC, 1,2,4-trichlorobenzene (140° C.): Mₙ=30.1 kg·mol⁻¹, M_{w}=66.0 kg·mol⁻¹, PDI=2.19.

### Example 10

OPV devices were built as previously described for polymer P2.

The following device performance for polymer P5 is obtained as described in table 3.

TABLE 3
Average open circuit potential (V_{oc}), current density
(J_{SC}), fill factor (FF), power conversion efficiency
(PCE) and best power conversion efficiency for specific
ratios of polymer P5 and PCBM-C₆₀.

| Ratio | | | | | |
|---|---|---|---|---|---|
| Polymer | conc. | Voc | Jsc | FF | PCE |
| P5:PCBM | mg · cm⁻³ | mV | mA · cm⁻² | % | % |
| 1.0:1.0 | 30 | 644 | −7.14 | 32.8 | 1.59 |
| 1.0:1.5 | 30 | 743 | −11.73 | 43.7 | 3.81 |
| 1.0:2.0 | 30 | 748 | −12.36 | 55.0 | 5.08 |
| 1.0:3.0 | 30 | 810 | −11.27 | 61.4 | 5.60 |

### Example 11

### Polymer P6

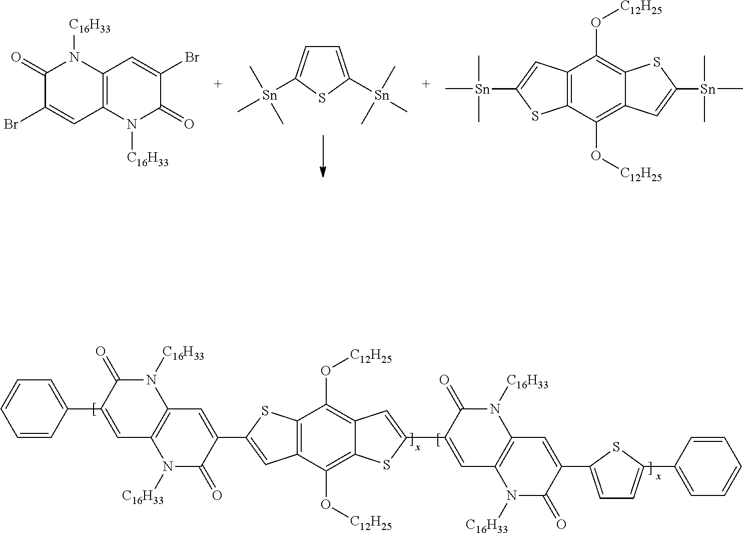

To a dry microwave vial is added 3,7-dibromo-1,5-dihexadecyl-1,5-dihydro-[1,5]naphthyridine-2,6-dione (312.4 mg, 0.41 mmol), 2,5-bis-trimethylstannanyl-thiophene (83.3 mg, 0.20 mmol), 4,8-didodecyloxy-2,6-bis-trimethylstannanyl-benzo[1,2-b;4,5-b′]dithiophene (179.7 mg, 0.20 mmol), Pd₂(dba)₃ (7.4 mg, 0.008 mmol) and tri-o-tolyl phosphine (9.9 mg, 0.033 mmol), the vial is evacuated and nitrogen purged (×3) and then degassed chlorobenzene (2.5 cm³) is added. The solution is degassed for a further 30 minutes before heating in a microwave reactor (Biotage Initiator) at 160° C. for 1 minute, 170° C. for 1 minute and at 180° C. for 30 minutes. The reaction mixture is then end-capped with tributylphenyl-stannane (0.13 ml, 0.40 mmol), heated to 180° C. for 10 minutes, and then bromobenzene (0.06 ml, 0.61 mmol) is added and the reaction mixture heated to 180° C. for a further 10 minutes. The reaction mixture is allowed to cool to 65° C. and precipitated into methanol. The solids are collected by filtration and purified via sequential Soxhlet extraction with acetone, petroleum ether 40-60 and cyclohexane. The cyclohexane fraction is then reduced in vacuo and redissolved in chloroform, then precipitated into methanol to yield a black polymer (0.31 g, 82%).

GPC, chlorobenzene (50° C.): Mₙ=12.7 kg·mol⁻¹, M_{w}=25.0 kg·mol⁻¹, PDI=1.97.

### Example 12

OPV devices were built as previously described for polymer P2.

The following device performance for polymer P6 is obtained as described in table 4.

TABLE 4
Average open circuit potential (V_{oc}), current density
(J_{SC}), fill factor (FF), power conversion efficiency
(PCE) and best power conversion efficiency for specific
ratios of polymer P6 and PCBM-C₆₀.

| Ratio | | | | | |
|---|---|---|---|---|---|
| Polymer | conc. | Voc | Jsc | FF | PCE |
| P6:PCBM | mg · cm⁻³ | mV | mA · cm⁻² | % | % |
| 1.0:1.0 | 30 | 762 | −1.28 | 46.9 | 0.46 |
| 1.0:1.5 | 30 | 776 | −1.31 | 49.6 | 0.51 |
| 1.0:2.0 | 30 | 782 | −1.39 | 44.9 | 0.49 |
| 1.0:3.0 | 30 | 783 | −1.56 | 50.7 | 0.62 |

### Example 13

### Polymer P7

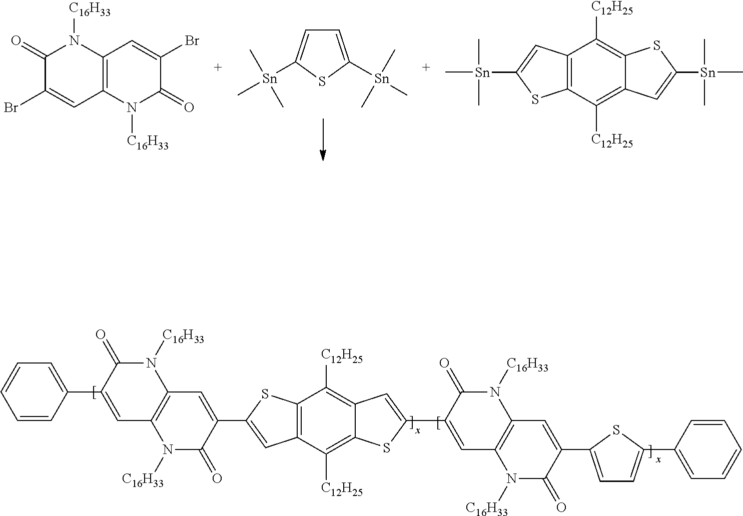

To a dry microwave vial is added 3,7-dibromo-1,5-dihexadecyl-1,5-dihydro-[1,5]naphthyridine-2,6-dione (497.3 mg, 0.65 mmol), 2,5-bis-trimethylstannanyl-thiophene (132.5 mg, 0.32 mmol), 4,8-didodecyl-2,6-bis-trimethylstannanyl-benzo[1,2-b;4,5-b′]dithiophene (275.7 mg, 0.32 mmol), Pd₂(dba)₃ (11.8 mg, 0.013 mmol) and tri-o-tolyl phosphine (15.8 mg, 0.052 mmol), the vial is evacuated and nitrogen purged (×3) and then degassed chlorobenzene (4.0 cm³) is added. The solution is degassed for a further 15 minutes before heating at 140° C. for 2 hours, then in a microwave reactor (Biotage Initiator) at 160° C. for 1 minute, 170° C. for 1 minute and at 180° C. for 30 minutes. N,N-Dimethylformamide (0.4 cm³) is added and the reaction heated at 180° C. for 1 minute, 190° C. for 1 minute and at 200° C. for 28 minutes. The reaction mixture is then end-capped with tributylphenyl-stannane (0.21 ml, 0.65 mmol), heated to 180° C. for 10 minutes, and then bromobenzene (0.10 ml, 0.97 mmol) is added and the reaction mixture heated to 180° C. for a further 10 minutes. The reaction mixture is allowed to cool to 65° C. and precipitated into methanol. The solids are collected by filtration and purified via sequential Soxhlet extraction with acetone, petroleum ether 40-60 and cyclohexane. The cyclohexane fraction is then reduced in vacuo and redissolved in chloroform, then precipitated into methanol to yield a black polymer (0.33 g, 55%).

GPC, chlorobenzene (50° C.): Mₙ=8.6 kg·mol⁻¹, M_{w}=17.0 kg·mol⁻¹, PDI=1.96.

### Example 14

OPV devices were built as previously described for polymer P2.

The following device performance for polymer P7 is obtained as described in table 5.

TABLE 5
Average open circuit potential (V_{oc}), current density
(J_{SC}), fill factor (FF), power conversion efficiency
(PCE) and best power conversion efficiency for specific
ratios of polymer P7 and PCBM-C₆₀.

| Ratio | | | | | |
|---|---|---|---|---|---|
| Polymer | conc. | Voc | Jsc | FF | PCE |
| P7:PCBM | mg · cm⁻³ | mV | mA · cm⁻² | % | % |
| 1.0:1.0 | 30 | 767 | −1.10 | 44.2 | 0.37 |
| 1.0:1.5 | 30 | 762 | −1.47 | 45.6 | 0.52 |
| 1.0:2.0 | 30 | 740 | −1.49 | 38.5 | 0.43 |
| 1.0:3.0 | 30 | 768 | −1.50 | 43.7 | 0.49 |

### Example 15

### Polymer P8

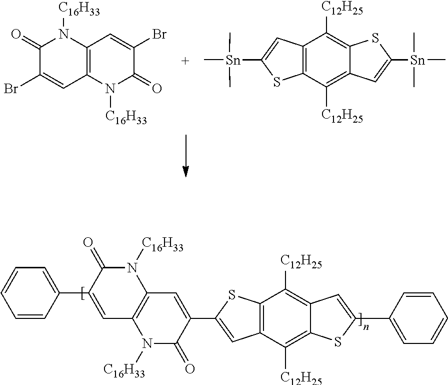

To a dry microwave vial is added 3,7-dibromo-1,5-dihexadecyl-1,5-dihydro-[1,5]naphthyridine-2,6-dione (395.8 mg, 0.52 mmol), 4,8-didodecyl-2,6-bis-trimethylstannanyl-benzo[1,2-b;4,5-b′]dithiophene (438.9 mg, 0.52 mmol), Pd₂(dba)₃ (9.4 mg, 0.010 mmol) and tri-o-tolyl phosphine (12.5 mg, 0.041 mmol), the vial is evacuated and nitrogen purged (×3) and then degassed chlorobenzene (4.0 cm³) and degassed N,N-dimethylformamide (1.0 cm³) is added. The solution is degassed for a further 15 minutes before heating in a microwave reactor (Biotage Initiator) at 160° C. for 1 minute, 170° C. for 1 minute and at 180° C. for 30 minutes. The reaction mixture is then end-capped with tributylphenyl-stannane (0.17 ml, 0.52 mmol), heated to 180° C. for 10 minutes, and then bromobenzene (0.08 ml, 0.77 mmol) is added and the reaction mixture heated to 180° C. for a further 10 minutes. The reaction mixture is allowed to cool to 65° C. and precipitated into methanol. The solids are collected by filtration and purified via sequential Soxhlet extraction with acetone, petroleum ether 40-60 and cyclohexane. The cyclohexane fraction is then reduced in vacuo and redissolved in chloroform, then precipitated into methanol to yield a black polymer (0.46 g, 79%).

GPC, chlorobenzene (50° C.): Mₙ=14.5 kg·mol⁻¹, M_{w}=28.1 kg·mol⁻¹, PDI=1.93.

### Example 16

OPV devices were built as previously described for polymer P2.

The following device performance for polymer P8 is obtained as described in table 6.

TABLE 6
Average open circuit potential (V_{oc}), current density
(J_{SC}), fill factor (FF), power conversion efficiency
(PCE) and best power conversion efficiency for specific
ratios of polymer P8 and PCBM-C₆₀.

| Ratio | | | | | |
|---|---|---|---|---|---|
| Polymer | conc. | Voc | Jsc | FF | PCE |
| P8:PCBM | mg · cm⁻³ | mV | mA · cm⁻² | % | % |
| 1.0:1.0 | 30 | 725 | −5.74 | 38.7 | 1.61 |
| 1.0:1.5 | 30 | 734 | −5.44 | 38.8 | 1.55 |
| 1.0:2.0 | 30 | 751 | −4.34 | 38.7 | 1.30 |
| 1.0:3.0 | 30 | 773 | −3.50 | 40.2 | 1.09 |

1. A compound comprising one or more divalent units of formula I 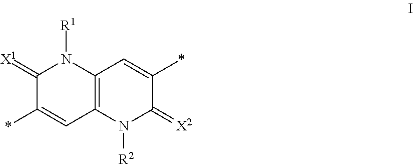
wherein
X¹ and X² independently of each other denote O or S,
R¹ and R² independently of each other denote H, straight-chain, branched or cyclic alkyl with 1 to 30 C atoms, in which one or more CH₂ groups are optionally replaced by —O—, —S—, —C(O)—, —C(S)—, —C(O)—O—, —O—C(O)—, —NR⁰, —SiR⁰R⁰⁰—, —CF₂—, —CHR⁰═cR⁰⁰—, —CY¹═CY²— or —C≡C— in such a manner that O and/or S atoms are not linked directly to one another, and in which one or more H atoms are optionally replaced by F, Cl, Br, I or CN, or denote aryl, heteroaryl, aryloxy or heteroaryloxy with 4 to 20 ring atoms which is optionally substituted, preferably by halogen or by one or more of the aforementioned alkyl or cyclic alkyl groups,
Y¹ and Y² are independently of each other H, F, Cl or CN,
R⁰ and R⁰⁰ are independently of each other H or optionally substituted C₁₋₄₀ carbyl or hydrocarbyl, and preferably denote H or alkyl with 1 to 12 C-atoms.

2. The compound according to claim 1, characterized in that, R¹ and R² denote straight-chain, branched or cyclic alkyl with 1 to 30 C atoms which is unsubstituted or substituted by one or more F atoms.

3. The compound according to claim 1, characterized in that X¹ and X² denote O.

4. The compound according to claim 1, characterized in that one of R¹ and R² is H and the other is different from H.

5. The compound according to claim 1, characterized in that it is a polymer comprising one or more units of formula I.

6. The polymer according to claim 5, characterized in that it comprises one or more units of formula IIa or IIb
—[(Ar¹)ₐ—(U)_{b}-(Ar²)_{c}—(Ar³)_{d}]— IIa
—[(U)_{b}-(Ar¹)ₐ—(U)_{b}-(Ar²)_{c}—(Ar³)_{d}]— IIb
wherein
U is a unit of formula I,
Ar¹, Ar², Ar³ are, on each occurrence identically or differently, and independently of each other, aryl or heteroaryl that is different from U, has 5 to 30 ring atoms and is optionally substituted, by one or more groups R^{S},
R^{S} is on each occurrence identically or differently F, Br, Cl, —CN, —NC, —NCO, —NCS, —OCN, —SCN, —C(O)NR⁰R⁰⁰, —C(O)X⁰, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl, carbyl or hydrocarbyl with 1 to 40 C atoms that is optionally substituted and optionally comprises one or more hetero atoms,
R⁰ and R⁰⁰ are independently of each other H or optionally substituted C₁₋₄₀ carbyl or hydrocarbyl,
X⁰ is halogen,
a, b, c are on each occurrence identically or differently 0, 1 or 2,
d is on each occurrence identically or differently 0 or an integer from 1 to 10,
wherein the polymer comprises at least one repeating unit of formula IIa or IIb wherein b is at least 1.

7. The polymer according to claim 5, characterized in that it additionally comprises one or more repeating units selected of formula IIIa or IIIb
—[(Ar¹)ₐ-(D)_{b}-(Ar²)_{c}—(Ar³)_{d}]— IIIa
-[(D)_{b}-(Ar¹)ₐ-(D)_{b}-(Ar²)_{c}—(Ar³)_{d}]— IIIb
wherein Ar¹, Ar², Ar³, are, on each occurrence identically or differently, and independently of each other, aryl or heteroaryl that is different from U, has 5 to 30 ring atoms and is optionally substituted, by one or more groups R^{S}, wherein U is a unit of formula I, and wherein R^{S} is on each occurrence identically or differently F, Br, Cl, —CN, —NC, —NCO, —NCS, —OCN, —SCN, —C(O)NR⁰R⁰⁰, —C(O)X°, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl, carbyl or hydrocarbyl with 1 to 40 C atoms that is optionally substituted and optionally comprises one or more hetero atoms, and a, b, and c are on each occurrence identically or differently 0, 1 or 2, and d is on each occurrence identically or differently 0 or an integer from 1 to 10, and D is an aryl or heteroaryl group that is different from U and Ar¹⁻³, has 5 to 30 ring atoms, is optionally substituted by one or more groups R^{S}, and is selected from aryl or heteroaryl groups having electron donor properties, wherein the polymer comprises at least one repeating unit of formula IIIa or IIIb wherein b is at least 1.

8. The polymer according to claim 5, characterized in that it is selected of formula IV: 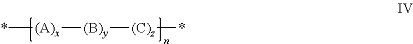
wherein
A, B, C independently of each other denote a distinct unit of formula I, IIa, IIb, IIIa, IIIb, or their preferred subformulae,
x is >0 and ≦1,
y is ≧0 and <1,
z is ≧0 and <1,
x+y+z is 1, and
n is an integer >1.

9. The polymer according to claim 5, characterized in that it is selected from the following formulae
*—[(Ar¹—U—Ar²)ₓ—(Ar³)_{y}]ₙ—* IVa
*—[(Ar¹—U—Ar²)ₓ—(Ar³—Ar³)_{y}]ₙ—* IVb
*—[(Ar¹—U—Ar²)ₓ—(Ar³—Ar³—Ar³)_{y}]ₙ—* IVc
*—[(Ar¹)ₐ—(U)_{b}-(Ar²)_{c}—(Ar³)_{d}]ₙ-* IVd
*—([(Ar¹)ₐ—(U)_{b}-(Ar²)_{c}—(Ar³)_{d}]ₓ—[(Ar¹)ₐ-(D)_{b}-(Ar²)_{c}—(Ar³)_{d}]_{y})ₙ—* IVe
*—[(U—Ar¹—U)ₓ—(Ar²—Ar³)_{y}]ₙ—* IVf
*—[(U—Ar¹—U)ₓ—(Ar²—Ar³—Ar²)_{y}]ₙ—* IVg
*—[(U)_{b}-(Ar¹)ₐ—(U)_{b}-(Ar²)_{c}]ₙ—* IVh
*—([(U)_{b}-(Ar¹)ₐ—(U)_{b}-(Ar²)_{c}]ₓ-[(D)_{b}-(Ar¹)ₐ-(D)_{b}-(Ar²)_{d}]_{y})ₙ—* IVi
*—[(U—Ar¹)ₓ—(U—Ar²)_{y}—(U—Ar³)_{z}]ₙ-* IVk
U is a unit of formula I,
Ar¹, Ar², Ar³ are, on each occurrence identically or differently, and independently of each other, aryl or heteroaryl that is different from U, has 5 to 30 ring atoms and is optionally substituted, by one or more groups R^{S},
R^{S} is on each occurrence identically or differently F, Br, Cl, —CN, —NC, —NCO, —NCS, —OCN, —SCN, —C(O)NR⁰R⁰⁰, —C(O)X°, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl, carbyl or hydrocarbyl with 1 to 40 C atoms that is optionally substituted and optionally comprises one or more hetero atoms,
R⁰ and R⁰⁰ are independently of each other H or optionally substituted C₁₋₄₀ carbyl or hydrocarbyl,
X⁰ is halogen, F, Cl or Br,
a, b, c are on each occurrence identically or differently 0, 1 or 2,
d is on each occurrence identically or differently 0 or an integer from 1 to 10,
D is an aryl or heteroaryl group that is different from U and Ar¹⁻³, has 5 to 30 ring atoms, is optionally substituted by one or more groups R^{S}
x is >0 and ≦1,
y is ≧0 and <1,
z is ≧0 and <1,
x+y+z is 1, and
n is an integer >1
wherein these polymers can be alternating or random copolymers, and wherein in formula IVd and IVe in at least one of the repeating units [(Ar¹)ₐ—(U)_{b}-(Ar²)_{c}—(Ar³)_{d}] and in at least one of the repeating units [(Ar¹)ₐ-(D)_{b}-(Ar²)_{c}—(Ar³)_{d}] b is at least 1 and wherein in formula IVh and IVi in at least one of the repeating units [(U)_{b}-(Ar¹)ₐ—(U)_{b}-(Ar²)_{d}] and in at least one of the repeating units [(U)_{b}-(Ar¹)ₐ—(U)_{b}-(Ar²)_{d}] b is at least 1.

10. The polymer according to claim 8, characterized in that it is selected of formula V
R⁵-chain-R⁶ V
wherein “chain” is a polymer chain selected of formulae IV R⁵ and R⁶ have independently of each other one of the meanings of R¹ or denote, independently of each other, H, F, Br, Cl, I, —CH₂Cl, —CHO, —CR′═CR″₂, —SiR′R″R″′, —SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′, or an endcap group, wherein X′ and X″ denote halogen, R′, R″ and R′″ have independently of each other one of the meanings of R⁰, and two of R′, R″ and R′″ may also form a ring together with the hetero atom to which they are attached.

11. The polymer according to claim 7, wherein one or more of D, Ar¹, Ar² and Ar³ denote aryl or heteroaryl selected from the group consisting of the following formulae 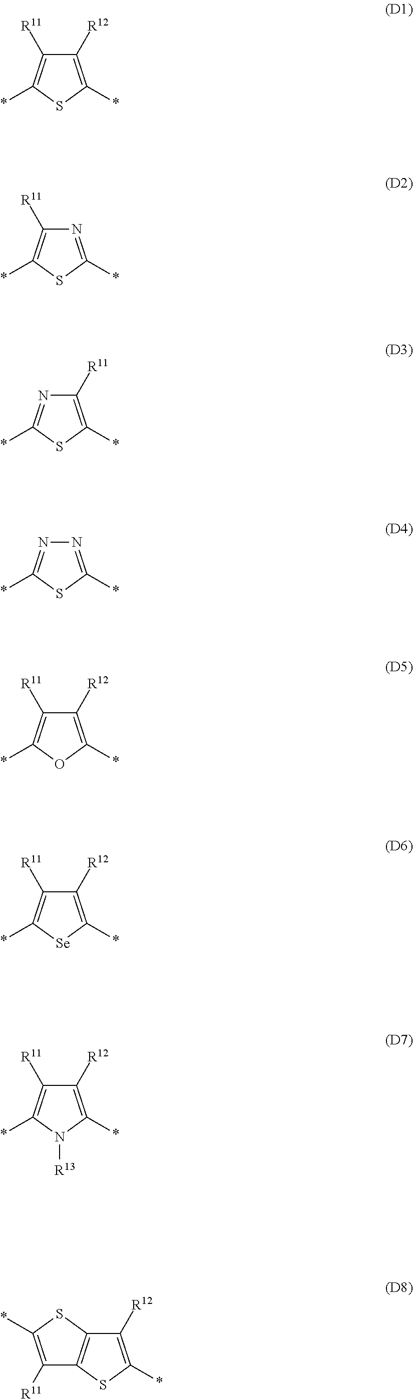 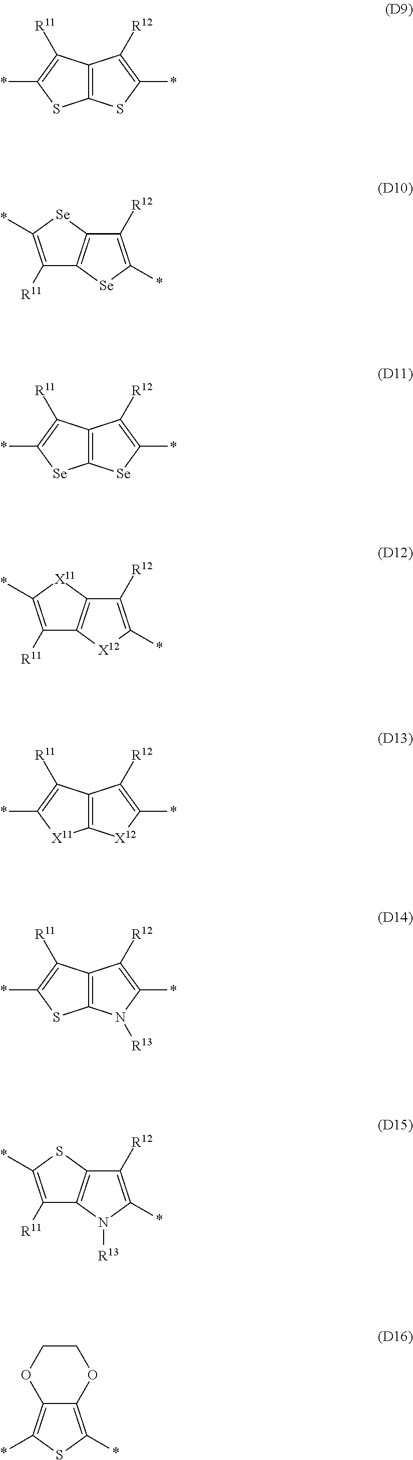 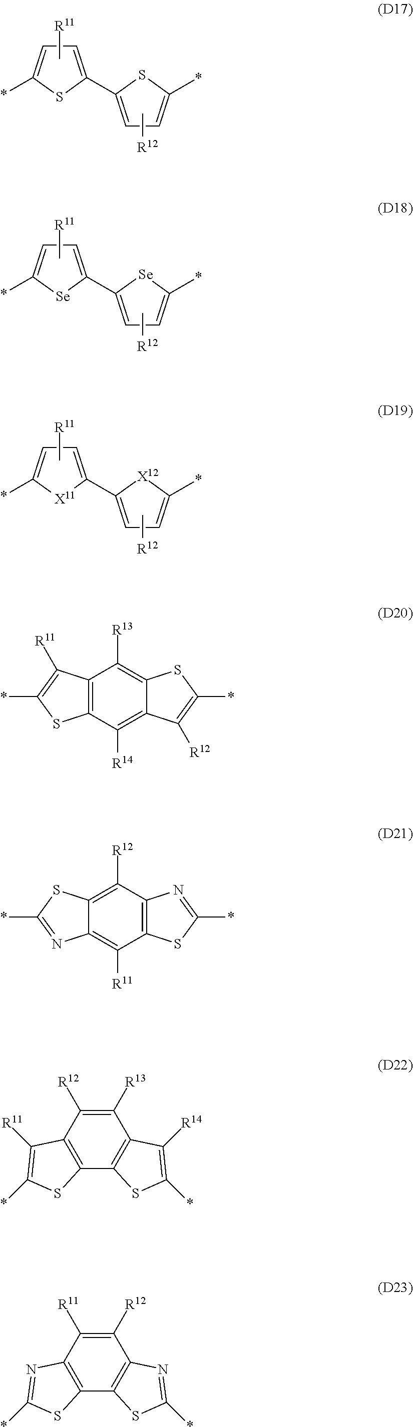 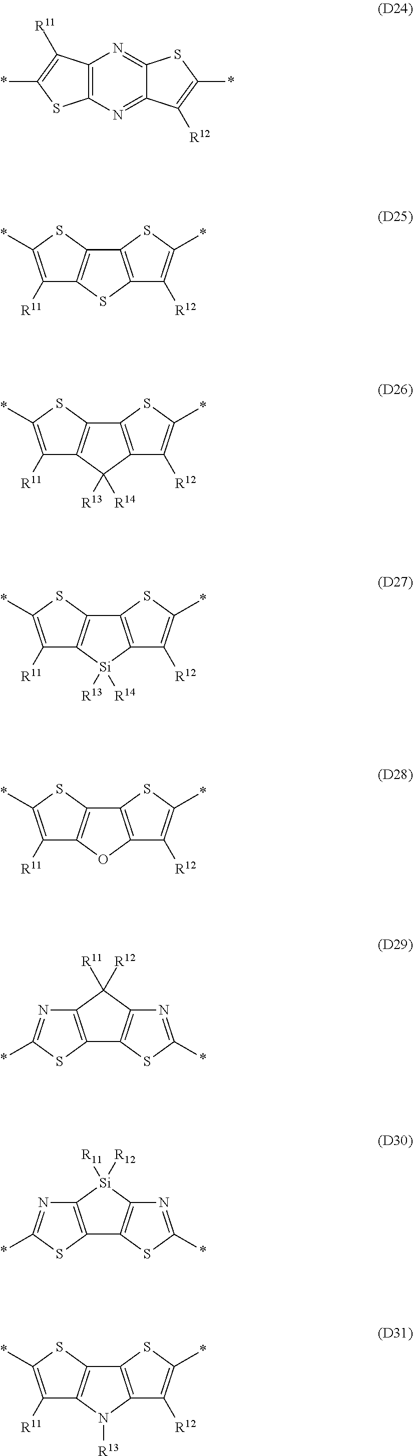 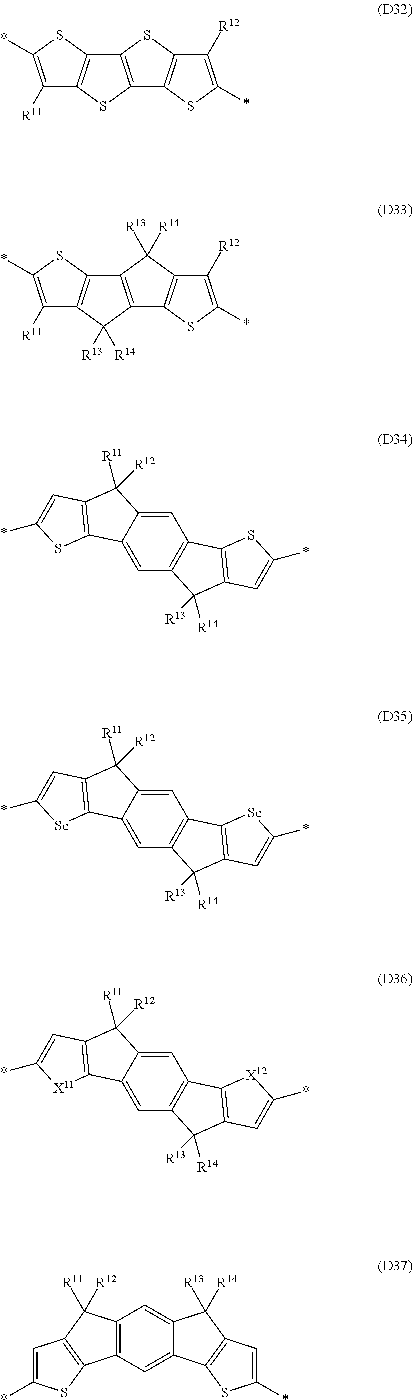 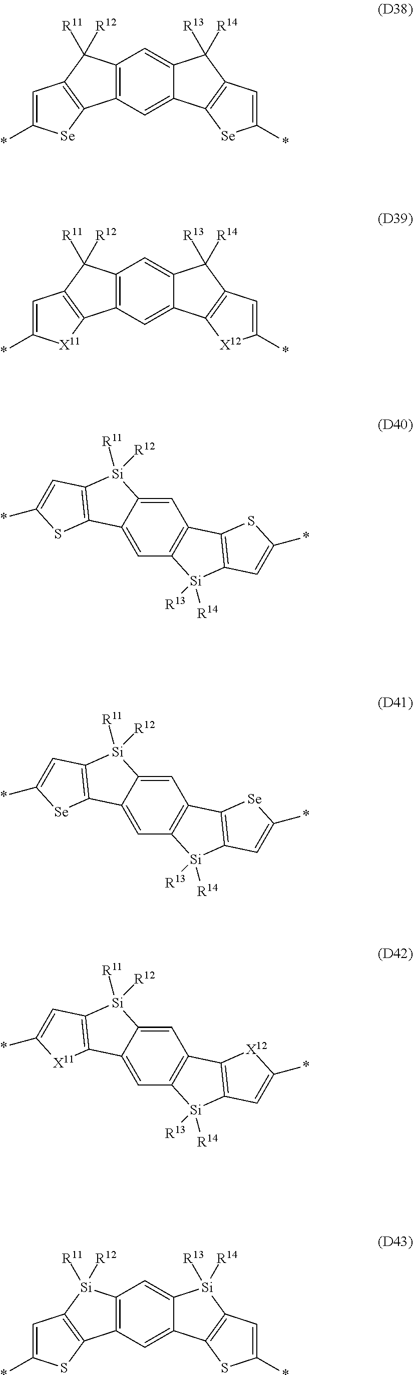 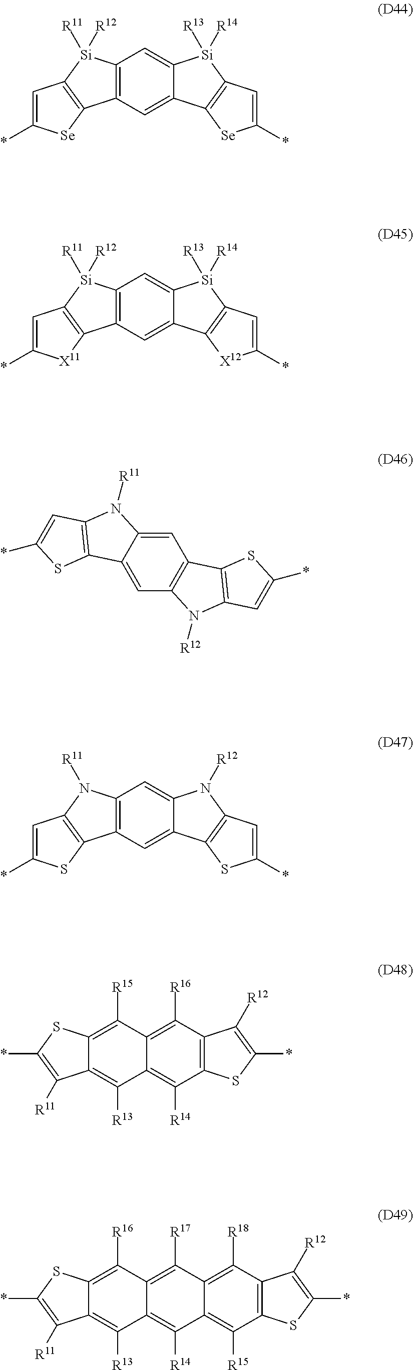 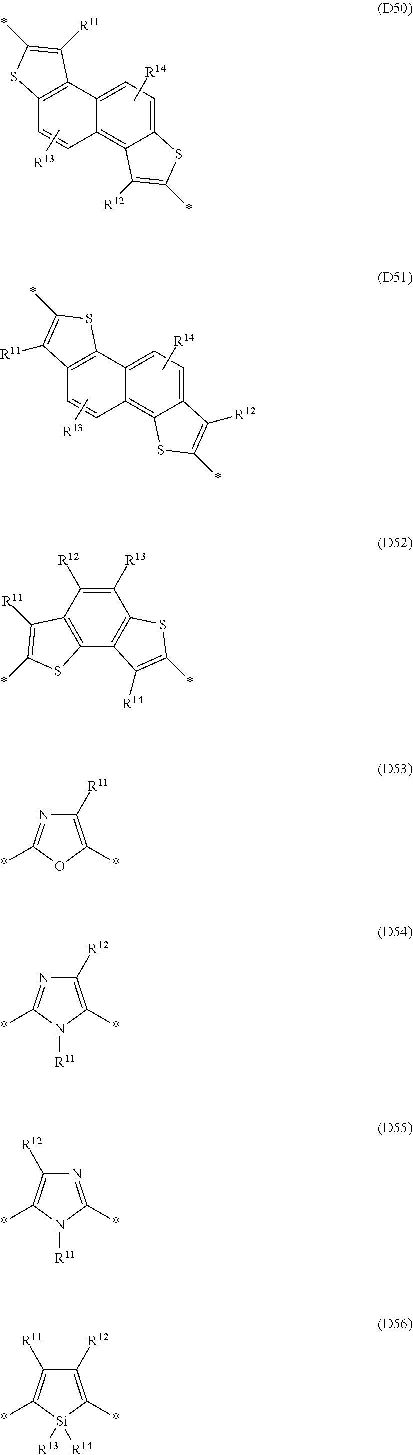 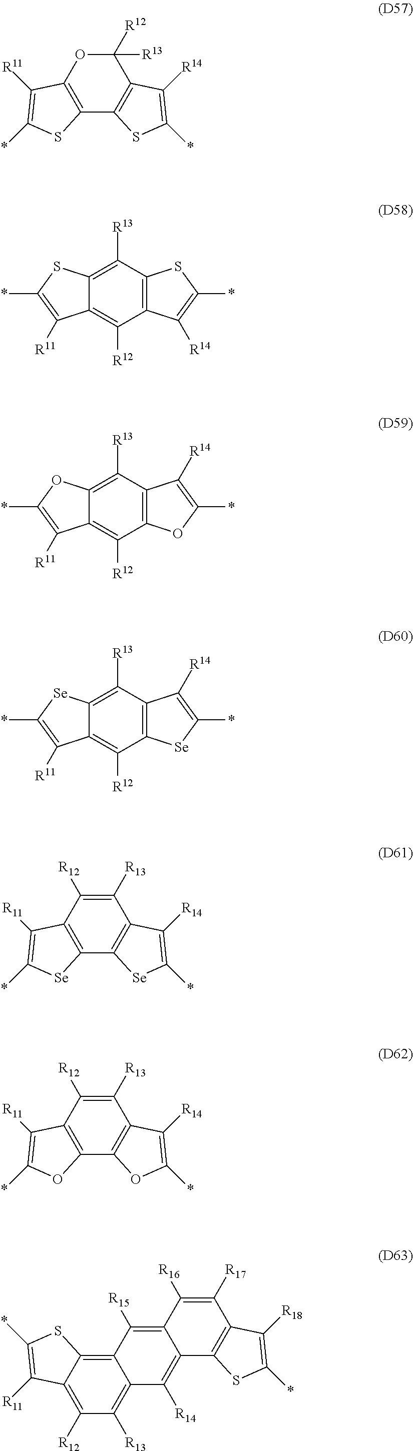
wherein one of X¹¹ and X¹² is S and the other is Se, and R¹¹, R¹², R¹³, R¹⁴, R¹⁵, R¹⁶, R¹⁷ and R¹⁸ independently of each other denote H or have one of the meanings of R¹.

12. The polymer according to claim 6, wherein Ar³ denotes aryl or heteroaryl selected from the group consisting of the following formulae 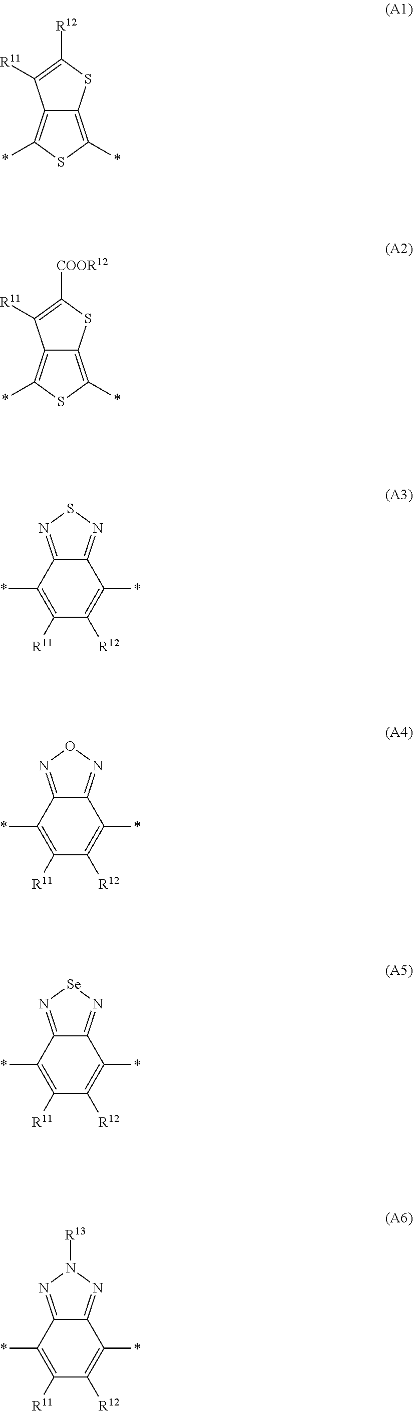 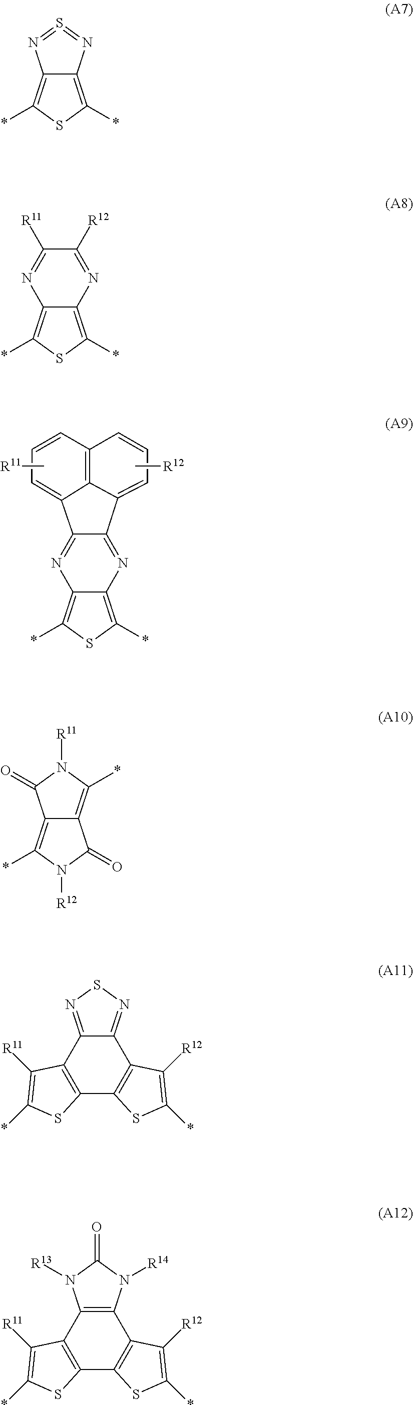 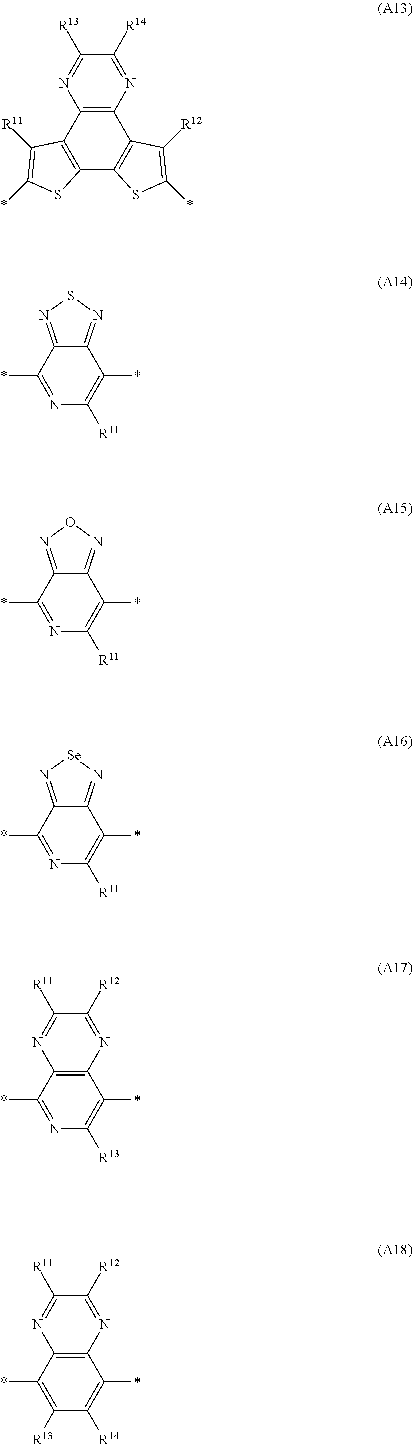 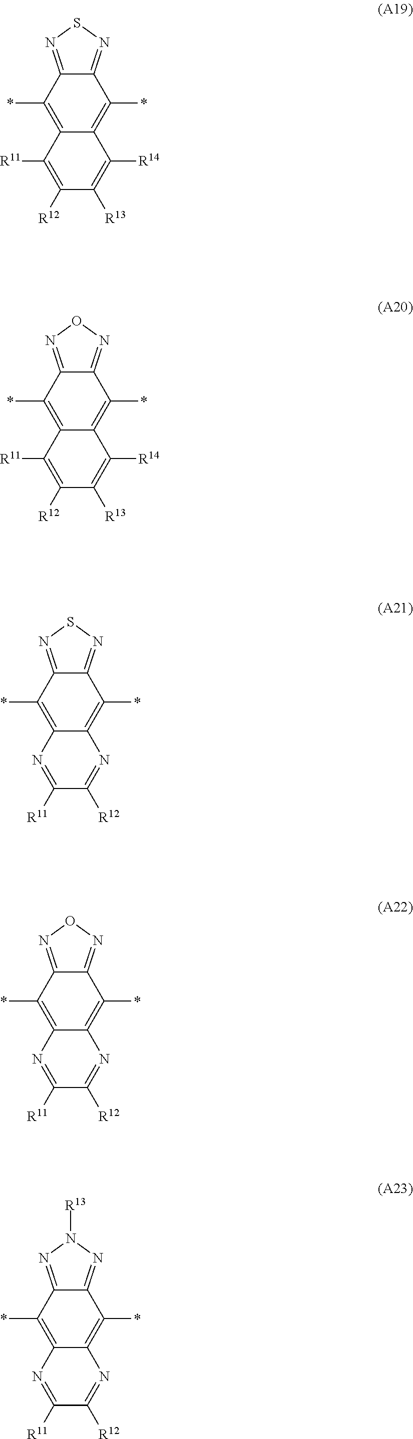 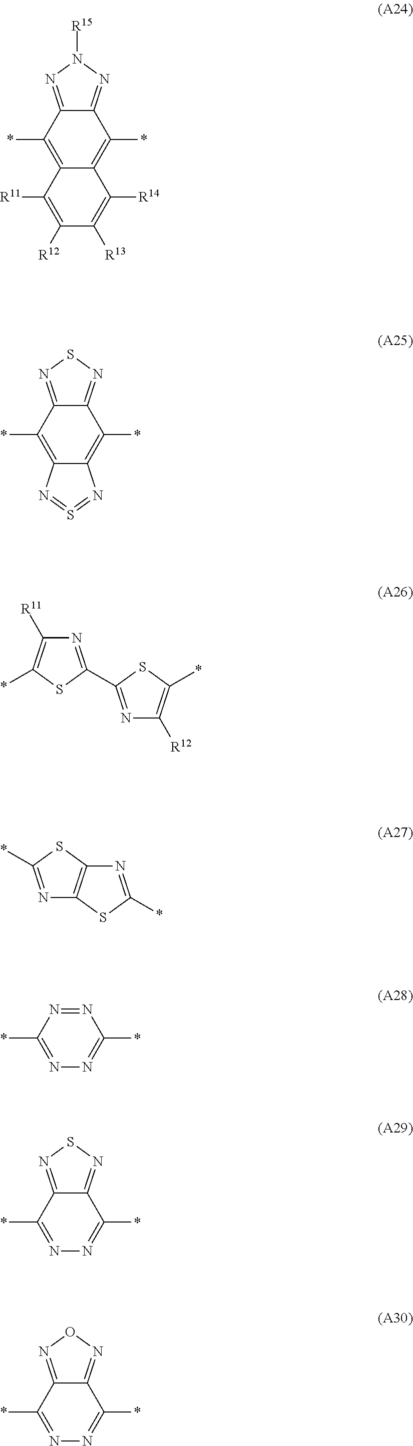 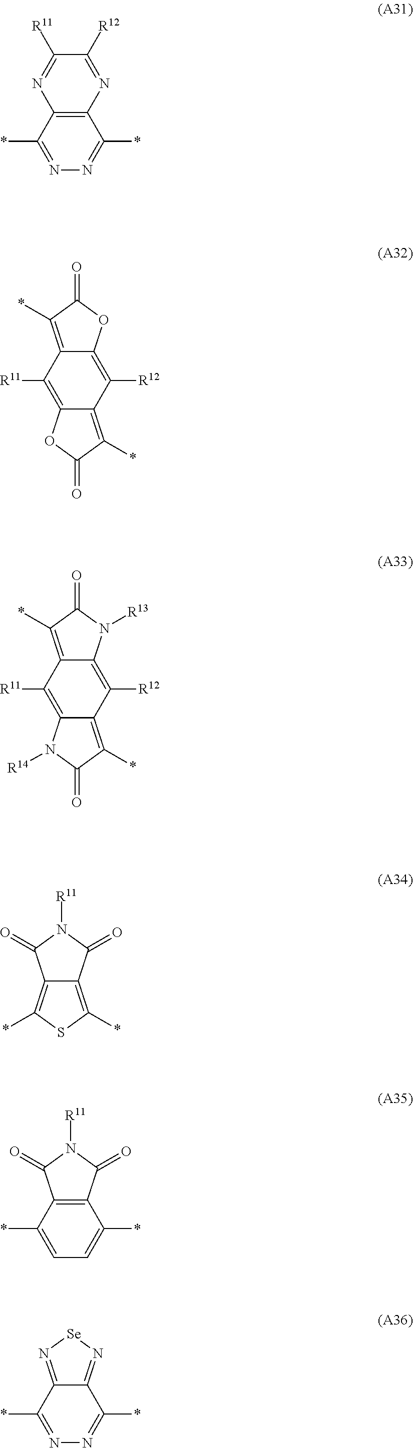 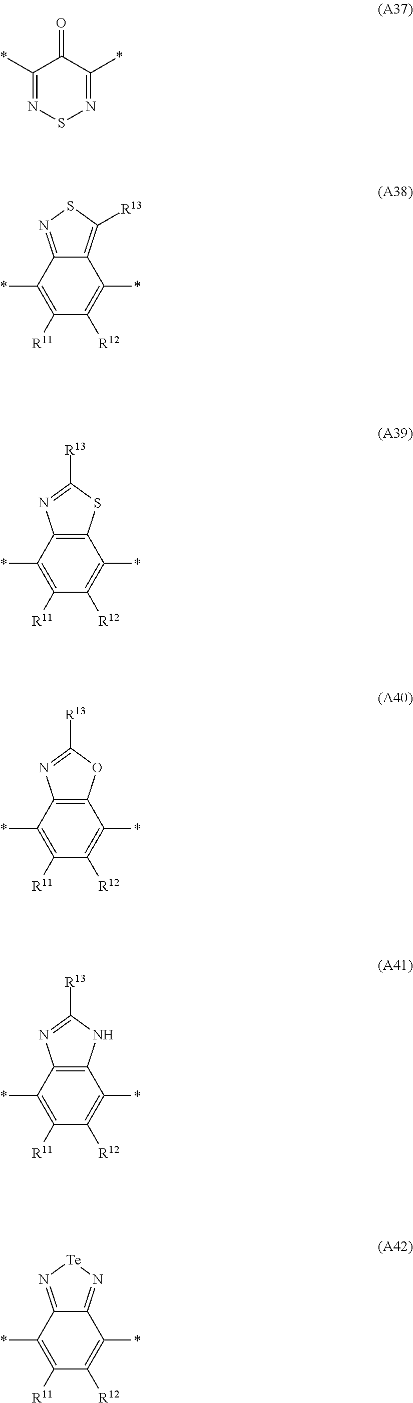 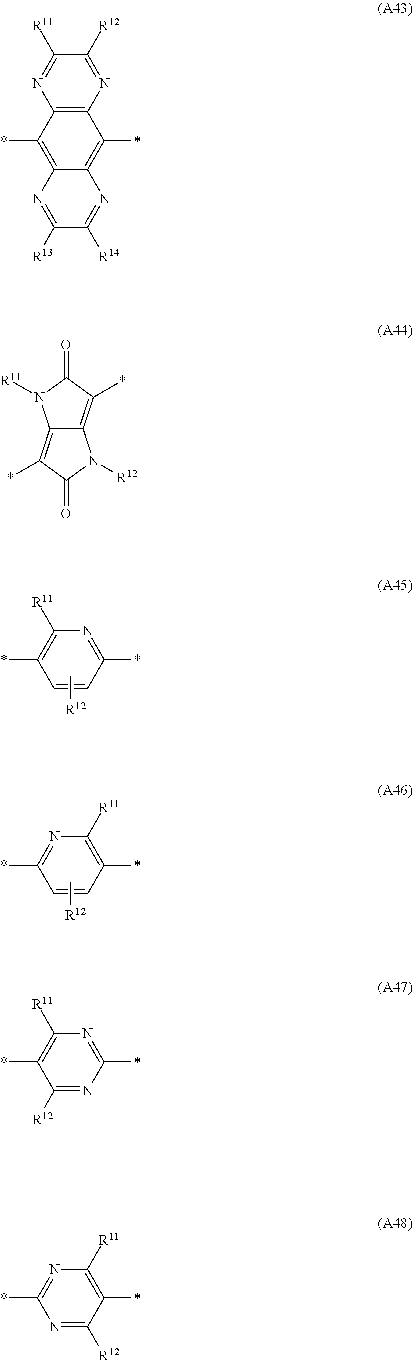 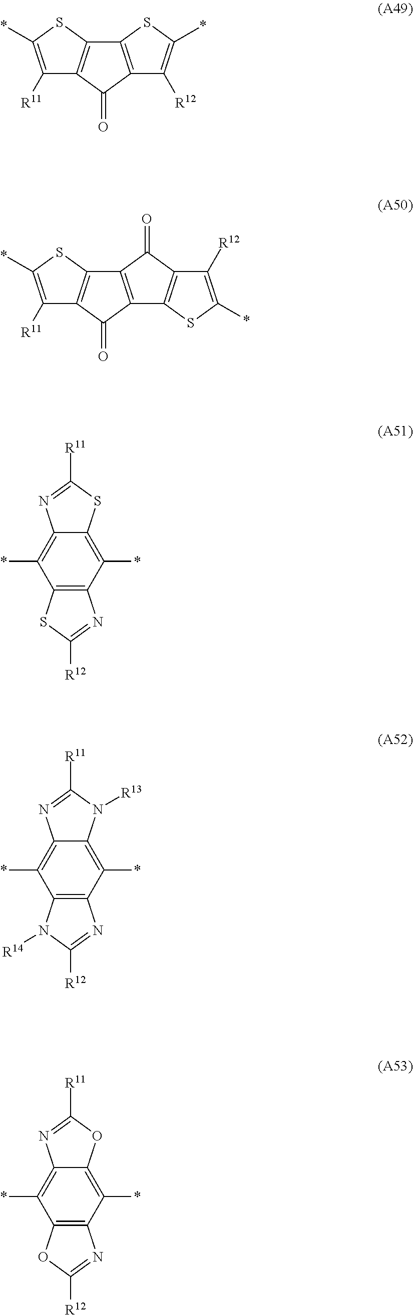 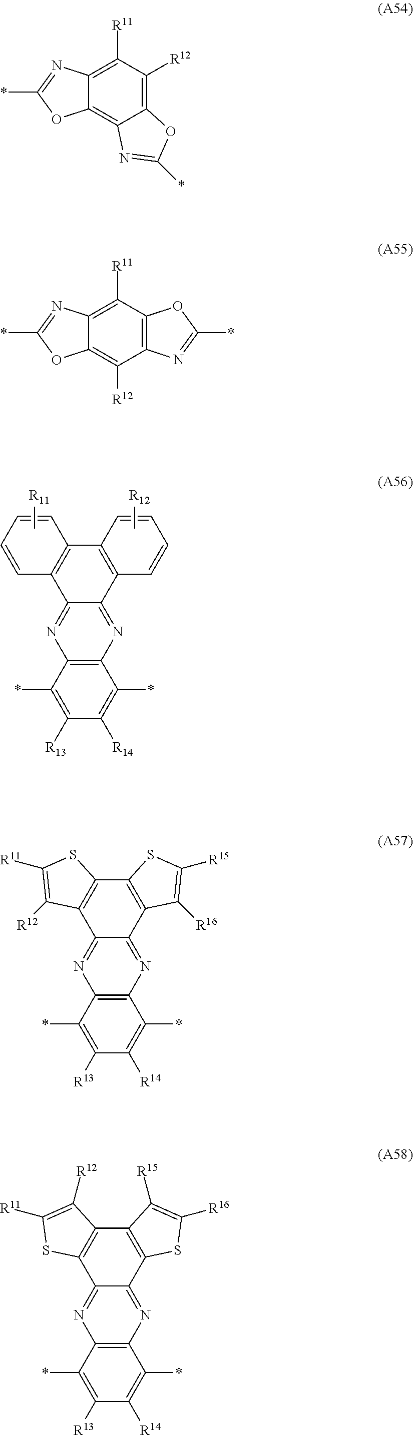 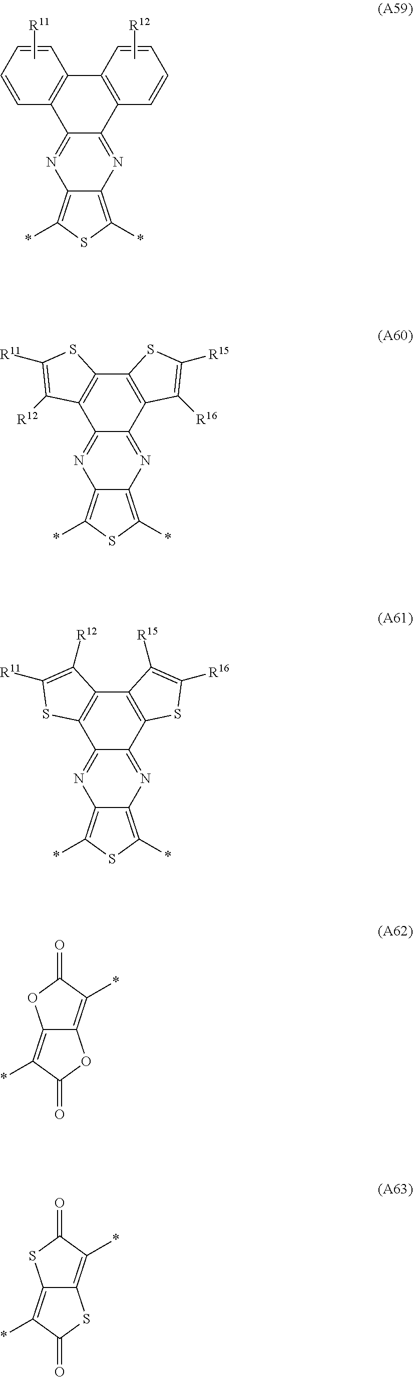 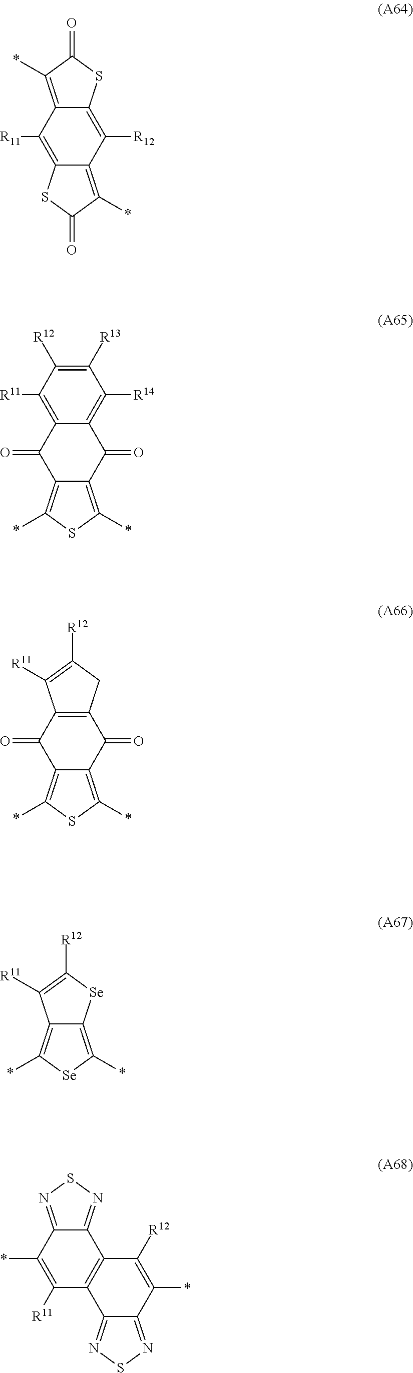
wherein one of X¹¹ and X¹² is S and the other is Se, and R¹¹, R¹², R¹³, R¹⁴ and R¹⁵ independently of each other denote H or have one of the meanings of R¹.

13. Polymer according to claim 5, wherein the polymer has a M_{w} of at least 5,000 and up to 300,000.

14. Polymer according to claim 6, wherein Ar¹, Ar², and Ar³ are independently of each other selected from the group consisting of

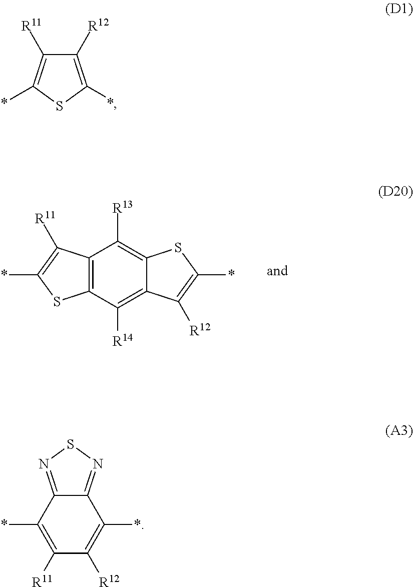

15. Polymer according to claim 6, wherein Ar¹, Ar², and Ar³ are independently of each other selected from the group consisting of 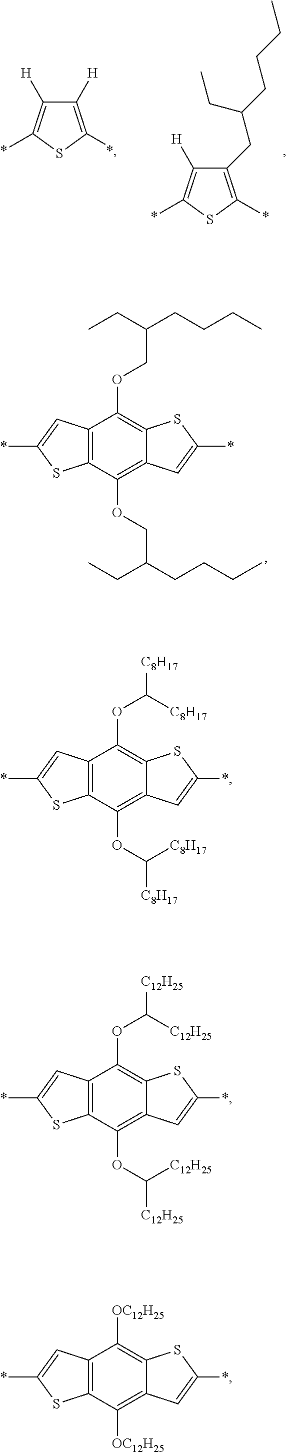 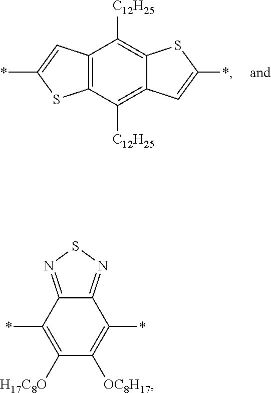
and R¹¹, R¹², R¹³, R¹⁴ and R¹⁵ independently of each other denote H or have one of the meanings of R¹.

16. The compound according to claim 1, characterized in that it is a small molecule of formula VII
R^{t1}-(Ar⁴)ₑ—(Ar⁵)_{f}—[(Ar⁶)_{g}—(Ar⁷)ₕ—U—(Ar⁸)ᵢ—(Ar⁹)ₖ]ₚ—(Ar¹⁰)ₗ—(Ar¹¹)ₒ—R^{t2} VII
wherein
U is a unit of formula I,
Ar⁴⁻¹² independently of each other denote —CY¹═CY²—, —C≡C—, or aryl or heteroaryl that has 5 to 30 ring atoms and is unsubstituted or substituted by one or more groups R¹, and one or more of Ar⁴⁻¹² may also denote U,
Y¹, Y² independently of each other denote H, F, Cl or CN,
R^{t1, t2} independently of each other denote H, F, Cl, Br, —CN, —CF₃, R, —CF₂—R, —O—R, —S—R, —SO₂—R, —SO³—R —C(O)—R, —C(S)—R, —C(O)—CF₂—R, —C(O)—OR, —C(S)—OR, —O—C(O)—R, —O—C(S)—R, —C(O)—SR, —S—C(O)—R, —C(O)NRR′, —NR′—C(O)—R, —NHR, —NRR′, —CR′═CR″R′″, —C≡C—R′, —C≡C—SiR′R″R′″, —SiR′R″R′″, —CH═C(CN)—C(O)—OR, —CH═C(COOR)₂, CH═C(CONRR′)₂, CH═C(CN)(Ar¹²), 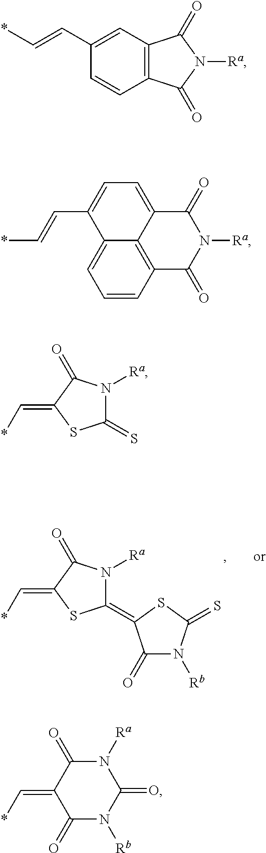
R^{a}, R^{b} are independently of each other aryl or heteroaryl, each having from 4 to 30 ring atoms and being unsubstituted or substituted with one or more groups R or R¹,
Ar¹² is aryl or heteroaryl, each having from 4 to 30 ring atoms and being unsubstituted or substituted with one or more groups R¹,
R is alkyl with 1 to 30 C atoms which is straight-chain, branched or cyclic, and is unsubstituted, substituted with one or more F or Cl atoms or CN groups, or perfluorinated, and in which one or more C atoms are optionally replaced by —O—, —S—, —C(O)—, —C(S)—, —SiR⁰R⁰⁰—, —NR⁰R⁰⁰—, —CHR⁰═CR⁰⁰— or —C≡C— such that O- and/or S-atoms are not directly linked to each other,
R⁰, R⁰⁰ independently of each other denote H or C₁₋₁₀ alkyl,
R′, R″, R′″ independently of each other have one of the meanings of R or denote H,
e, f, g, h, i, k, l, o are independently of each other 0 or 1, with at least one of e, f, g, h, i, k, l, o being 1,
p is 1, 2 or 3.

17. The compound according to claim 16, characterized in that Ar¹⁻¹² are selected from the following formulae: 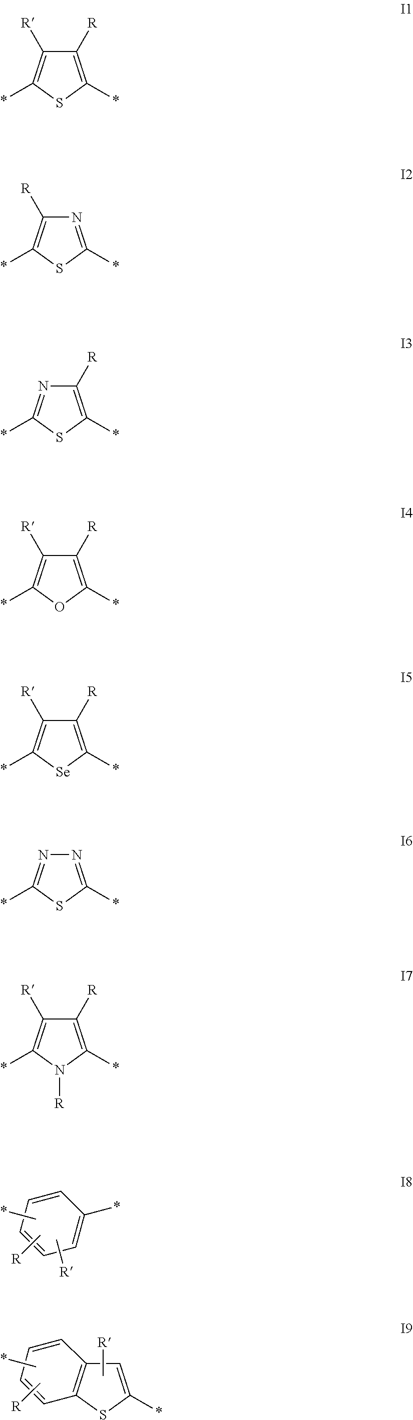 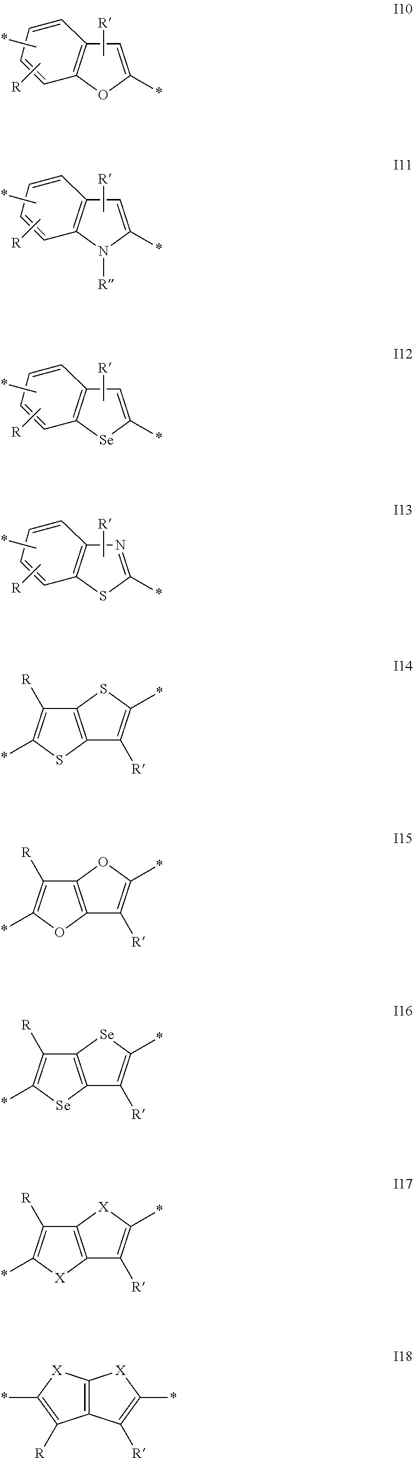 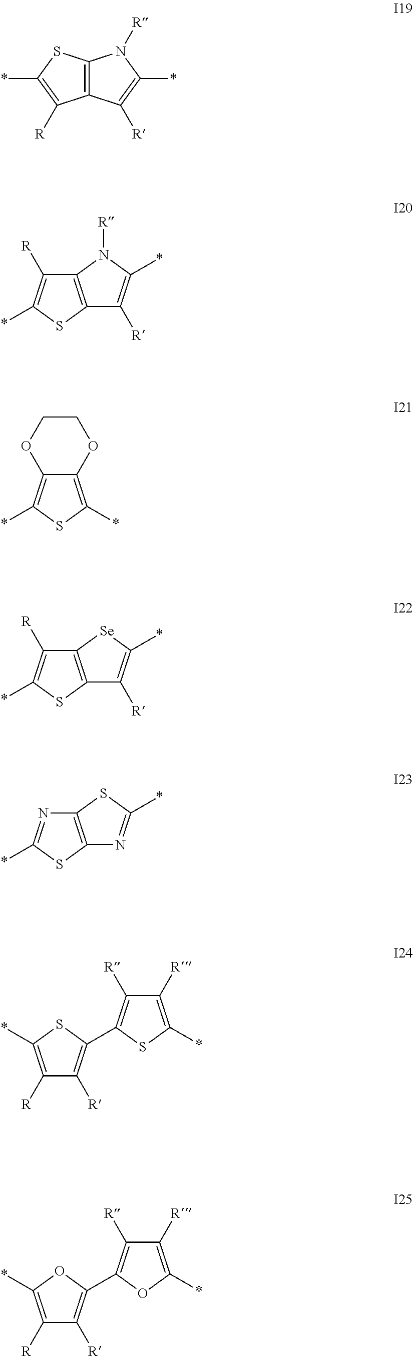 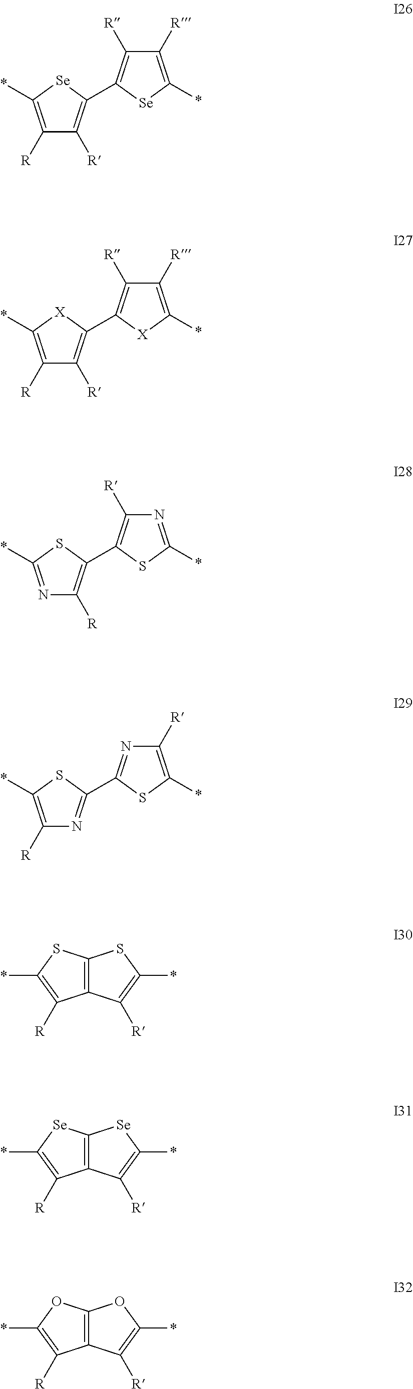 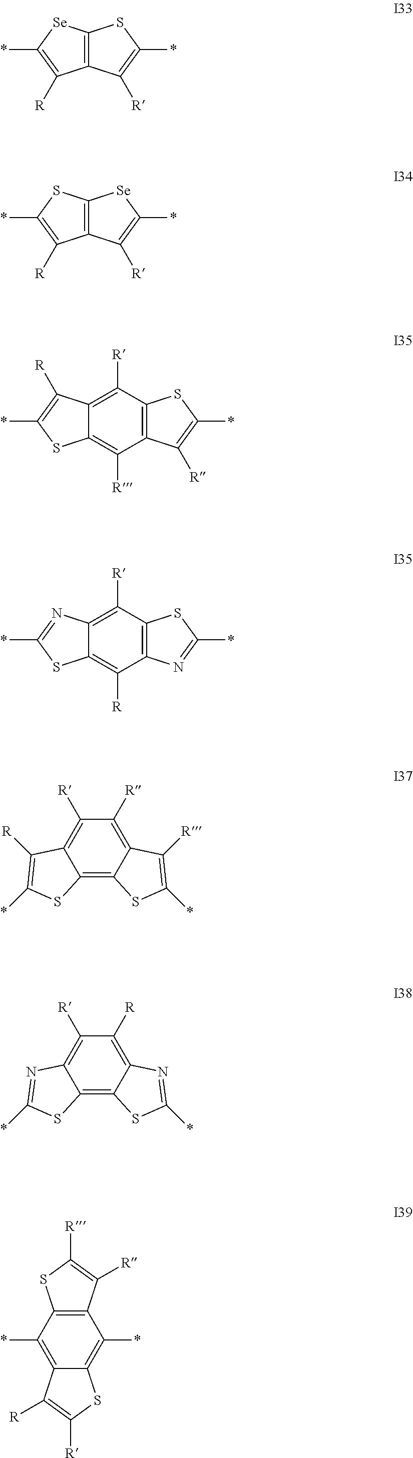 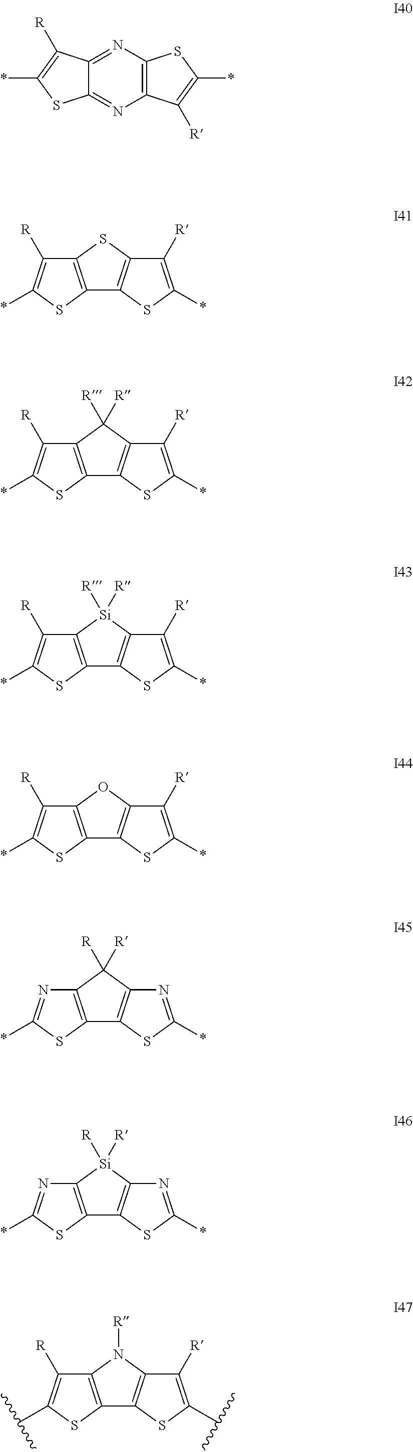 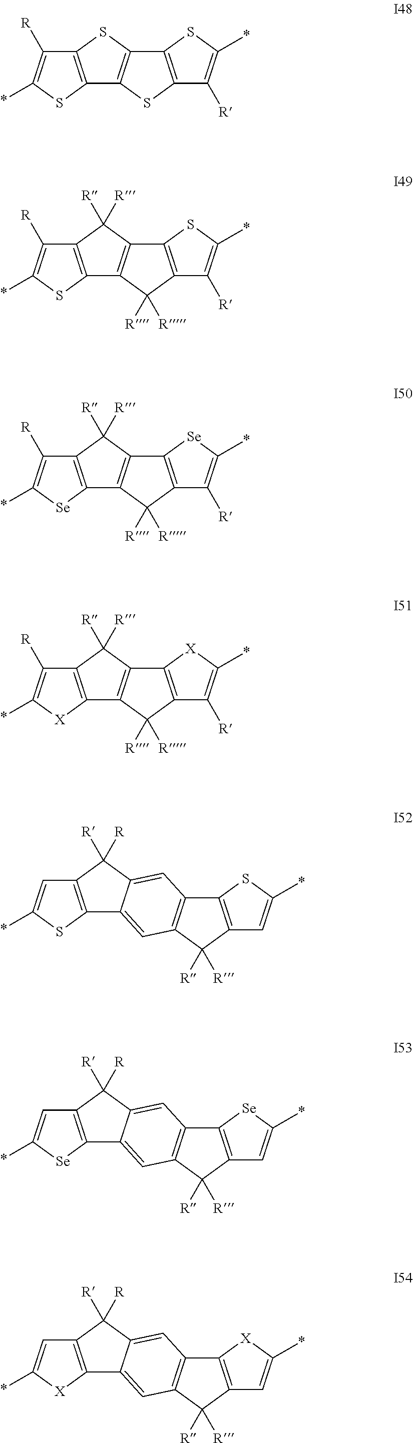 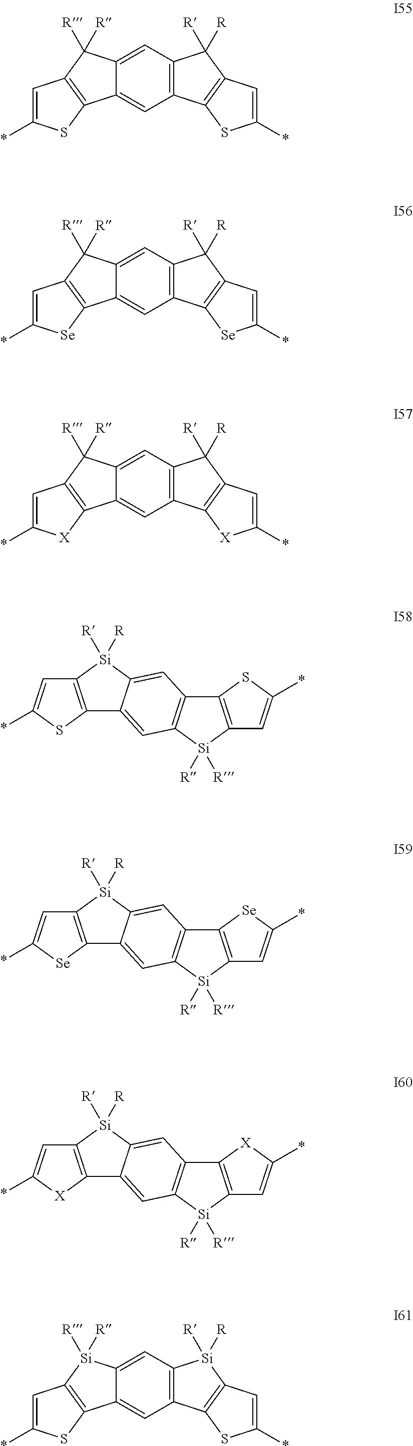 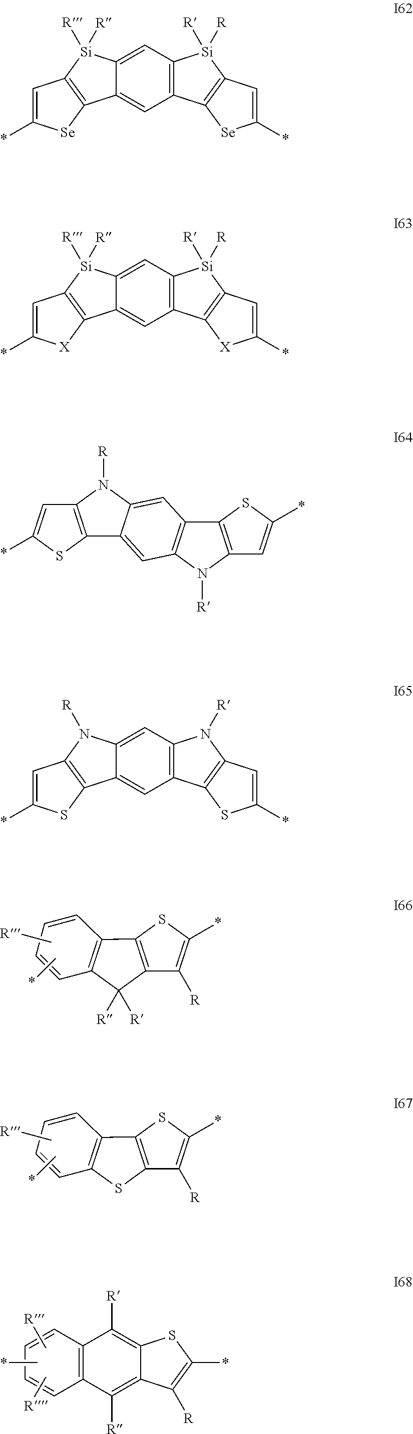 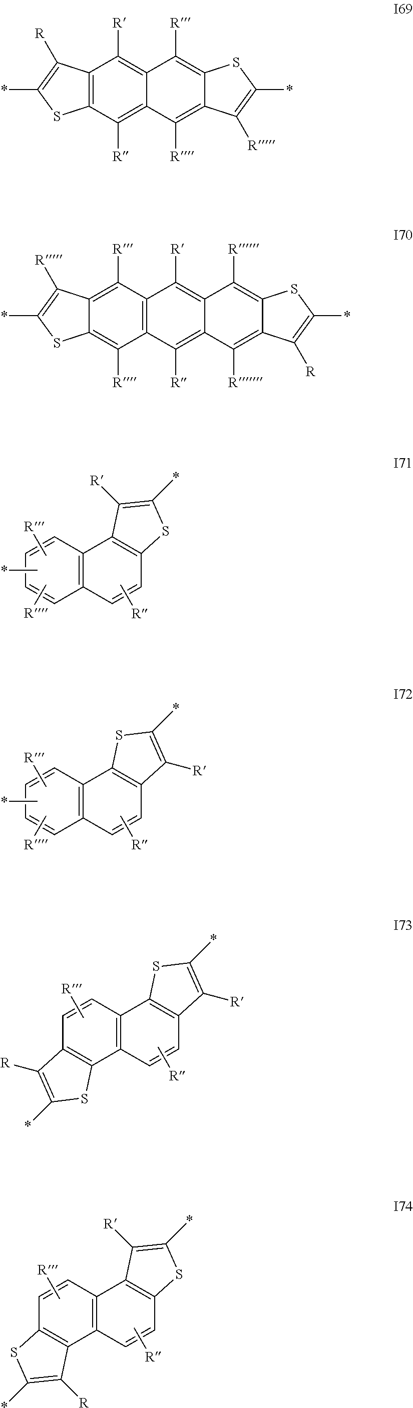 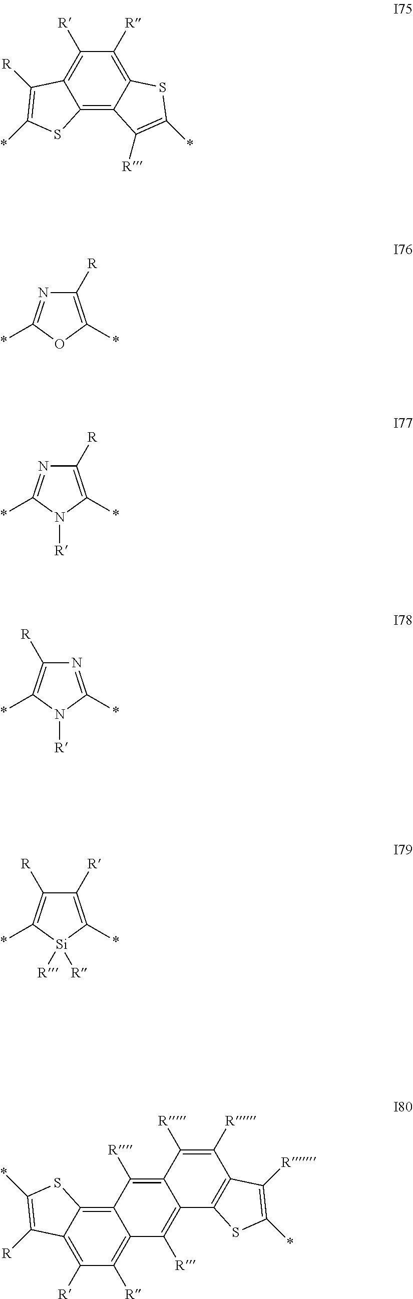
wherein R, R′, R″, R′″, R″″, R″″′, R″″″ and R″″″′ have one of the meanings of R¹.

18. Compound according to claim 1 comprising one or more units selected from the group consisting of the following formulae 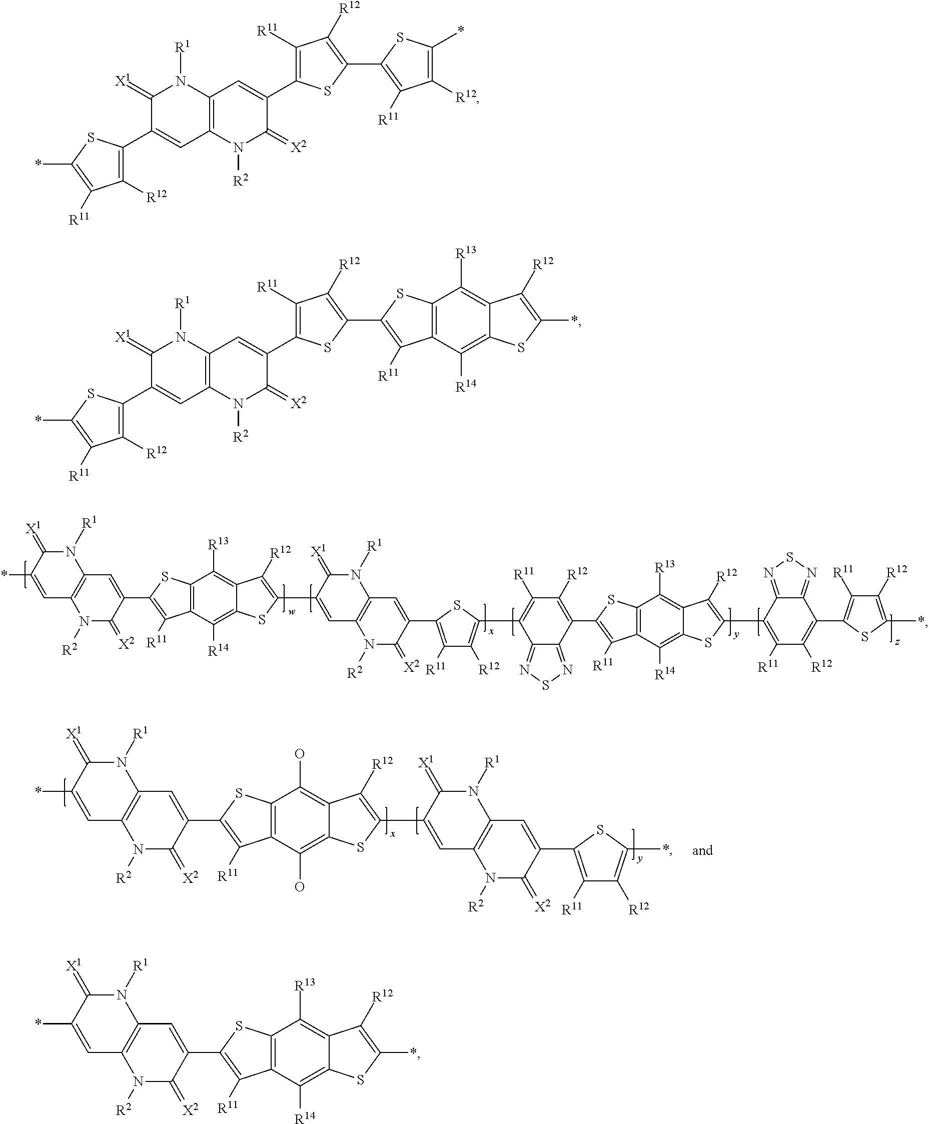
R¹¹, R¹², R¹³, and R¹⁴ independently of each other denote H or have one of the meanings of R¹
x is >0 and ≦1,
y is ≧0 and <1,
z is ≧0 and <1,
x+y+z is 1.

19. Compound according to claim 18 comprising one or more units selected from the group consisting of the following formulae

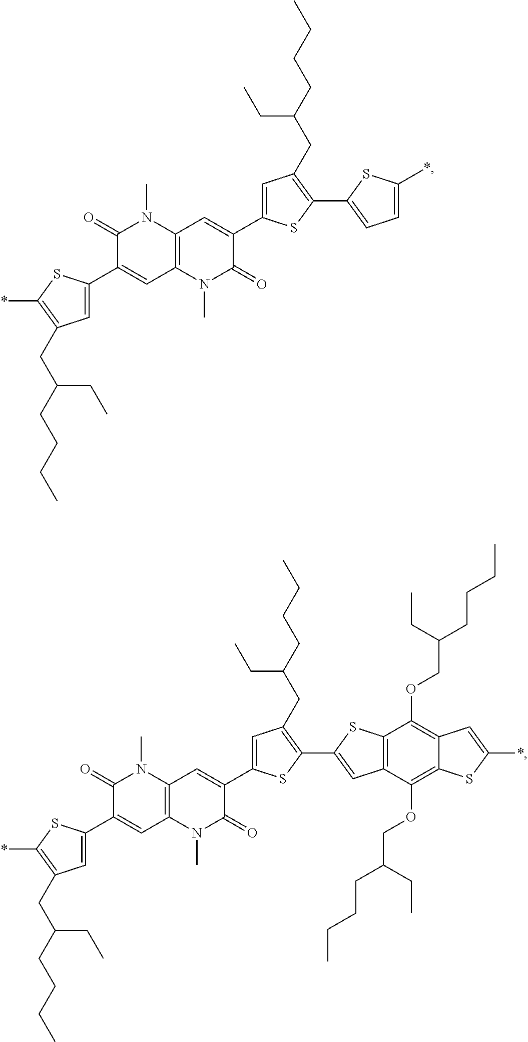

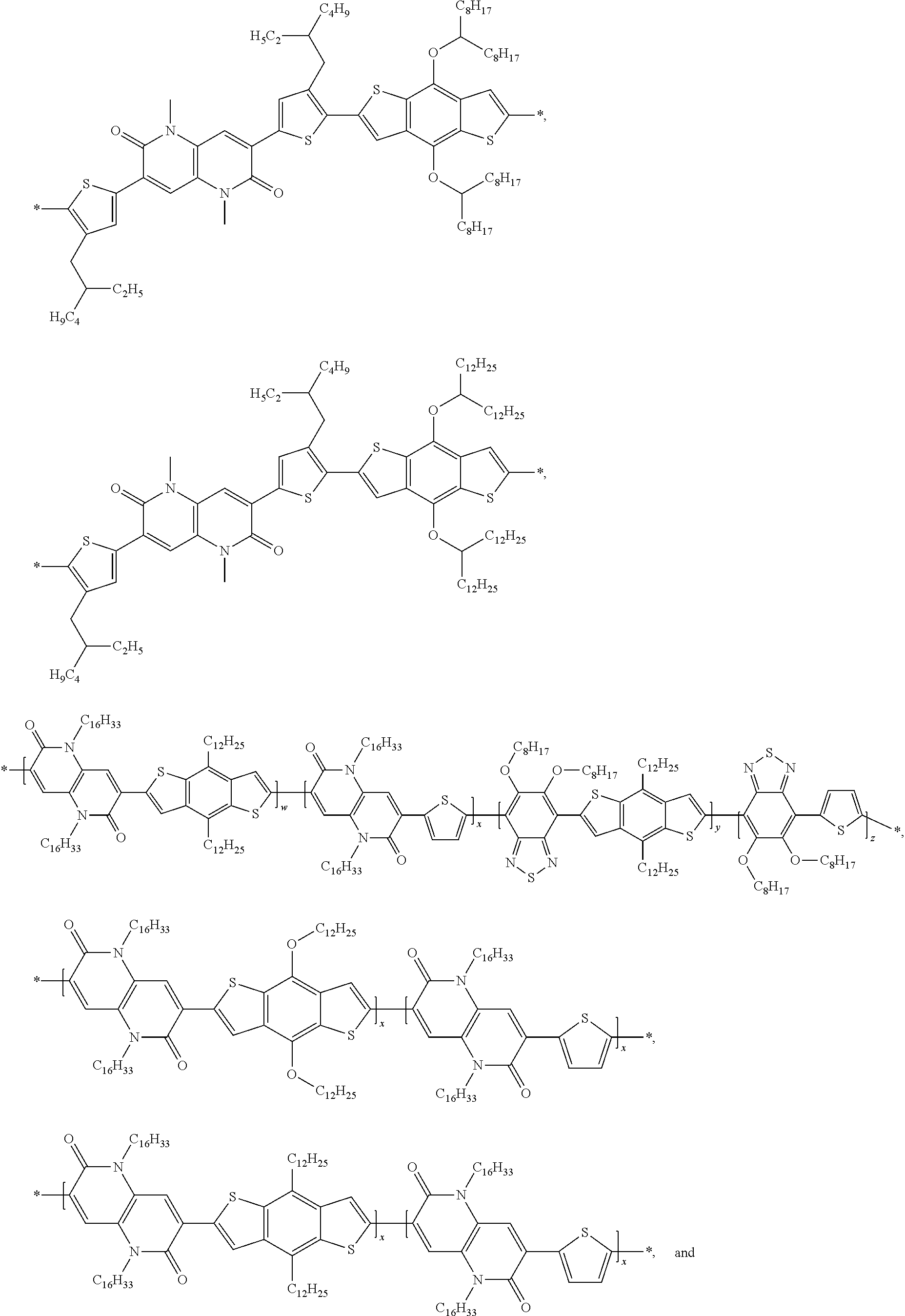

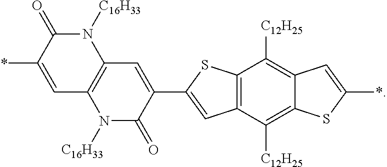

20. A mixture or polymer blend comprising one or more compounds according to claim 1 and one or more compounds or polymers having semiconducting, charge transport, hole/electron transport, hole/electron blocking, electrically conducting, photoconducting or light emitting properties.

21. A mixture or polymer blend comprising one or more compounds according to claim 1 and one or more compounds or polymers having semiconducting, charge transport, hole/electron transport, hole/electron blocking, electrically conducting, photoconducting or light emitting properties and further comprising one or more n-type organic semiconductor compounds.

22. The mixture or polymer blend according to claim 21, characterized in that the n-type organic semiconductor compound is a fullerene or substituted fullerene.

23. A formulation comprising one or more polymers, mixtures or polymer blends of a compound according to claim 1, and one or more solvents.

24. In an optical, electrooptical, electronic, electroluminescent or photoluminescent device, or in a component of such a device, or in an assembly comprising such a device or component, the improvement wherein the device or component contains a polymer, mixture, polymer blend or formulation of a polymer of a compound according to claim 1.

25. A charge transport, semiconducting, electrically conducting, photoconducting or light emitting material comprising a polymer, formulation, mixture or polymer blend according to claim 5.

26. An optical, electrooptical, electronic, electroluminescent or photoluminescent device, or a component thereof, or an assembly comprising it, which comprises a charge transport, semiconducting, electrically conducting, photoconducting or light emitting material, or comprises a polymer, mixture, polymer blend or formulation, according to claim 5.

27. A device, a component thereof, or an assembly comprising it according to claim 26, wherein the device is selected from organic field effect transistors (OFET), thin film transistors (TFT), organic light emitting diodes (OLED), organic light emitting transistors (OLET), organic photovoltaic devices (OPV), organic photodetectors (OPD), organic solar cells, laser diodes, Schottky diodes, photoconductors and photodetectors, the component is selected from charge injection layers, charge transport layers, interlayers, planarising layers, antistatic films, polymer electrolyte membranes (PEM), conducting substrates, conducting patterns, and the assembly is selected from integrated circuits (IC), radio frequency identification (RFID) tags or security markings or security devices containing them, flat panel displays or backlights thereof, electrophotographic devices, electrophotographic recording devices, organic memory devices, sensor devices, biosensors and biochips.

28. The device according to claim 27, which is an OFET, bulk heterojunction (BHJ) OPV device, inverted BHJ OPV device or OPD device.

29. A monomer of formula VIa or VIb
R⁷—(Ar¹)ₐ—U—(Ar²)_{c}—R⁸ VIa
R⁷—U—(Ar¹)ₐ—U—R⁸ VIb
wherein a and c are each independently 0, 1, or 2, U is a unit of formula I, Ar¹ and Ar² are each independently aryl or heteroaryl with 5-30 ring atoms, optionally substituted by R^{s}, which is on each occurrence identically or differently F, Br, Cl, —CN, —NC, —NCO, —NCS, —OCN, —SCN, —C(O)NR⁰R⁰⁰, —C(O)X⁰, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl, carbyl or hydrocarbyl with 1 to 40 C atoms that is optionally substituted and optionally comprises one or more hetero atoms R⁷ and R⁸ are selected from the group consisting of Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ and —Sn(Z⁴)₃, wherein X⁰ is halogen, Z¹⁻⁴ are selected from the group consisting of alkyl and aryl, each being optionally substituted, and two groups Z² may also together form a cyclic group.

30. The monomer according to claim 29, which is selected from the following formulae
R⁷—Ar¹—U—Ar²—R⁸ VI1
R⁷—U—R⁸ VI2
R⁷—Ar¹—U—R⁸ VI3
R⁷—U—Ar²—R⁸ VI4
R⁷—U—Ar¹—U—R⁸ VI5

31. A process of preparing a polymer according to claim 5 comprising coupling one or more monomers of formula VIa or VIb
R⁷—(Ar¹)ₐ—U—(Ar²)_{c}—R⁸ VIa
R⁷—U—(Ar¹)ₐ—U—R⁸ VIb
wherein a and c are each independently 0, 1, or U is a unit of formula I, Ar¹ and Ar² are each independently aryl or heteroaryl with 5-30 ring atoms, optionally substituted by R^{s}, which is on each occurrence identically or differently F, Br, Cl, —CN, —NC, —NCO, —NCS, —OCN, —SCN, —C(O)NR⁰R⁰⁰, —C(O)X°, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl, carbyl or hydrocarbyl with 1 to 40 C atoms that is optionally substituted and optionally comprises one or more hetero atoms wherein R⁷ and R⁸ are selected from Cl, Br, I, —B(OZ²)₂ and —Sn(Z⁴)₃, with each other and/or with one or more monomers selected from the following formulae
R⁷—(Ar¹)ₐ-D-(Ar²)_{c}—R⁸ VIII
R⁷—Ar¹—R⁸ IX
R⁷—Ar³—R⁸ X
wherein Ar³ is aryl or heteroaryl with 5-30 ring atoms optionally substituted by R^{S}, which is on each occurrence identically or differently F, Br, Cl, —CN, —NC, —NCO, —NCS, —OCN, —SCN, —C(O)NR⁰R⁰⁰, —C(O)X⁰, —C(O)R⁰, —NH₂, —NR⁰R⁰⁰, —SH, —SR⁰, —SO₃H, —SO₂R⁰, —OH, —NO₂, —CF₃, —SF₅, optionally substituted silyl, carbyl or hydrocarbyl with 1 to 40 C atoms that is optionally substituted and optionally comprises one or more hetero atoms,
R⁰ and R⁰⁰ are independently of each other H or optionally substituted C₁₋₄₀ carbyl or hydrocarbyl,
X⁰ is halogen,
D is an aryl or heteroaryl group that is different from U and Ar¹⁻³, has 5 to 30 ring atoms, is optionally substituted by one or more groups R^{S}, in an aryl-aryl coupling reaction.
